(19) United States
(12) Patent Application Publication
(10) Pub. No.: US 2025/0250216 A1
(43) Pub. Date: Aug. 7, 2025

(54) CYCLIC COMPOUND HAVING IODINE ATOM

(71) Applicant: Mitsubishi Gas Chemical Company, Inc., Tokyo (JP)

(72) Inventors: Masahiro MATSUMOTO, Niigata-shi, Niigata (JP); Masataka IINUMA, Niigata-shi, Niigata (JP); Tadashi OMATSU, Hiratsuka-shi, Kanagawa (JP); Takashi SATO, Hiratsuka-shi, Kanagawa (JP); Masatoshi ECHIGO, Chiyoda-ku, Tokyo (JP)

(21) Appl. No.: 18/855,112

(22) PCT Filed: Apr. 10, 2023
(86) PCT No.: PCT/JP2023/015256

§ 371 Date: Oct. 8, 2024

(30) Foreign Application Priority Data
Apr. 8, 2022 (JP) 2022-064331

(51) Int. Cl. C07C 29/62; C07C 43/23; C07C 45/63; C07C 67/28; G03F 7/004

(52) U.S. Cl. CPC C07C 29/62; C07C 43/23; C07C 45/63; C07C 67/28; G03F 7/0045

(57) ABSTRACT

A compound represented by the following formula (1):

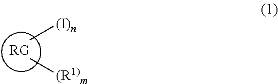

wherein RG is a group containing at least one cyclic structure,
I is an iodine atom,
R¹ is a monovalent functional group having 0 to 30 carbon atoms, containing no polymerizable unsaturated bond, and being optionally the same or different;
n is an integer of 1 to 5, and
m is an integer of 1 to 5.

### TECHNICAL FIELD

The present invention relates to a cyclic compound having an iodine atom.

### BACKGROUND ART

In recent years, in the production of semiconductor elements and liquid crystal display elements, semiconductors (patterns) and pixels have been rapidly miniaturized due to the advance in lithography technology. Thus, a material for corresponding to miniaturization is desired. For example, Patent Literature 1 discloses a resist composition having a structure in which a plurality of aromatic rings are crosslinked, and containing a compound having an iodine atom. The resist composition is excellent in etching resistance. Patent Literature 2 discloses a compound having a polymerizable group and an iodine atom. The resist composition containing the compound is considered to form a resist pattern excellent in CD uniformity.

### CITATION LIST

### Patent Literature

Patent Literature 1: International Publication No. WO 2020/040161
Patent Literature 2: Japanese Patent Laid-Open No. 2021-188040

### SUMMARY OF INVENTION

### Technical Problem

The development of a lithography composition capable of corresponding to miniaturization, preferably a compound useful for a resist composition is desired. In view of such circumstances, the present invention has an object to provide a compound useful as a lithography composition.

### Solution to Problem

The inventors have found out that a compound having a specific structure can solve the above problems. More specifically, the problem can be solved by the following present inventions.

### Aspect 1

A compound represented by the formula (1), which will be mentioned later.

### Aspect 2

A composition for lithography comprising the compound according to the aspect 1.

### Aspect 3

The composition for lithography according to the aspect 2, comprising two or more kinds of compounds represented by the formula (1).

### Aspect 4

A composition comprising the compound according to the aspect 1.

### Aspect 5

The composition according to the aspect 4, further comprising a compound represented by the formula (DM0-1) or the formula (BP0-1), which will be mentioned later, or a combination thereof.

### Aspect 6

The composition according to the aspect 5, wherein the compound represented by the formula (DM0-1) is a compound represented by the formula (DM1a), (Dn1), or (Da1), which will be mentioned later, and the compound represented by the formula (BP0-1) is a compound represented by the following formula (BP1a), (BP2a), (Bn1), or (Ba1), which will be mentioned later.

### Aspect 7

The composition according to the aspect 5, comprising the compound represented by the formula (DM0-1).

### Aspect 8

The composition according to any one of the aspects 5 to 7, wherein the compounds represented by the formula (1) and the formula (DM0-1) satisfy the following relationship:

0.1≥[an amount of the compound of the formula (DM0-1)]/[an amount of the compound of the formula (1)]≥0.000001.

### Aspect 9

The composition according to any one of the aspects 5 to 8, wherein the compound represented by the formula (DM0-1) is the compound represented by the formula (DM1a), the formula (Dn1), or the formula (Da1).

### Aspect 10

The composition according to the aspect 5, comprising the compound represented by the formula (BP0-1).

### Aspect 11

The composition according to the aspect 5 or 10, wherein the compound represented by the formula (BP0-1) is a compound represented by the formula (BP1a), the formula (BP2a), the formula (Bn1), or the formula (Ba1).

### Aspect 12

The composition according to the aspect 5, 10, or 11, wherein the compound represented by the formula (BP0-1) is a compound represented by the formula (BP1a) wherein Z is not I, or a compound represented by the formula (BP2a), the formula (Bn1), or the formula (Ba1).

### Aspect 13

The composition according to any one of the aspects 5 to 12, wherein the compounds represented by the formula (1), the formula (DM0-1), and the formula (BP0-1) satisfy the following relational expression:

0.1≥([a total amount of the compound of the formula (DM0-1) and the compound of the formula (BP0-1)])/[an amount of the compound of the formula (1)]≥0.000001.

### Aspect 14

The composition according to any one of the aspects 2 to 13, wherein RG in the formula (1) is a group derived from a benzene ring optionally having a substituent.

### Aspect 15

The composition according to any one of the aspects 2 to 13, wherein RG in the formula (1) is a group derived from a naphthalene ring optionally having a substituent.

### Aspect 16

The composition for lithography according to any one of the aspects 2 to 13, wherein RG in the formula (1) is a group derived from an adamantane ring optionally having a substituent.

### Aspect 17

The composition according to any one of the aspects 2 to 16, wherein a content of metal impurities is less than 1 ppm.

### Aspect 18

A method for producing the compound according to any one of the aspects 1 and 24 to 48, comprising a step of introducing an iodine atom or an R¹ group into the compound containing the RG group.

### Aspect 19

A method for producing the compound according to any one of the aspects 1, 24, 25, 28 to 33, 36, and 43, that is a method for producing the compound represented by formula (1), wherein
the compound is represented by the formula (Bz), which will be mentioned later, the method comprising:
1) a step of providing a compound represented by the formula (MB), which will be mentioned later;
2) an iodination step of iodinating the compound;
3) a protective group introduction step of introducing a protective group into the compound; and
4) a reduction step of reducing the compound.

### Aspect 20

A method for producing the compound according to any one of the aspects 1, 24, 25, 28 to 31, 34, 36, and 43, that is a method for producing the compound represented by formula (1), wherein
the compound is represented by the formula (N), which will be mentioned later, the method comprising:
1) a step of providing a compound represented by the formula (MN):
2) an iodination step of iodinating the compound;
3) a protective group introduction step of introducing a protective group into the compound; and
4) a reduction step of reducing the compound.

### Aspect 21

A method for producing the compound according to any one of the aspects 1, 24, 26, 27, 29 to 31, 35, 36, and 43, that is a method for producing the compound represented by formula (1), wherein
the compound is represented by the formula (Ad), which will be mentioned later, the method comprising:
1) a step of providing a compound represented by the formula (MA), which will be mentioned later; and
2) an iodination step of iodinating the compound.

### Aspect 22

A method for expressing a sensitizing effect in radiation irradiation of a composition for lithography using the compound according to any one of the aspects 1 and 24 to 48.

### Aspect 23

The method according to the aspect 22, wherein two or more kinds of the compounds are used.

### Aspect 24

The compound according to the aspect 1, wherein
RG is a group derived from benzene, naphthalene, anthracene, pyrene, a heteroaromatic compound, or a polycyclic alicyclic compound, optionally having a substituent; and
R¹ is composed of
one or more R^{f′} selected from the group consisting of a hydroxy group, and an ether group having a protective group eliminatable by acid, alkali, or heat, and
zero or more hydrocarbon groups R^{g} having 0 to 30 carbon atoms and optionally containing a substituent.

### Aspect 25

The compound according to the aspect 1 or 24, wherein
RG is a group derived from benzene or naphthalene, the group optionally having a substituent; and
R¹ is composed of
one or more R^{f} selected from the group consisting of a hydroxy group, and an ether group having a protective group, and
zero or more hydrocarbon groups R^{g} having 0 to 30 carbon atoms and optionally containing a substituent.

### Aspect 26

The compound according to the aspect 1, wherein
RG is a group derived from a polycyclic alicyclic compound optionally having a substituent; and
R¹ is composed of
one or more R^{f′} selected from the group consisting of a hydroxy group, and an ether group having a protective group eliminatable by acid, alkali, or heat, and
zero or more hydrocarbon groups R^{g} having 0 to 30 carbon atoms and optionally containing a substituent.

### Aspect 27

The compound according to the aspect 1 or 26, wherein
RG is a group derived from a polycyclic alicyclic compound optionally having a substituent; and
R¹ is composed of
one or more R^{f} selected from the group consisting of a hydroxy group, and an ether group having a protective group, and
zero or more hydrocarbon groups R^{g} having 0 to 30 carbon atoms and optionally containing a substituent.

### Aspect 28

The compound according to the aspect 1, 24, or 25, wherein
when RG is a group containing a benzene ring and a plurality of R¹ is present, R¹ contains no combination of an alkoxy group (excluding one having a protective group) and an aldehyde group, no combination of the alkoxy group and a hydroxy group, and no combination of an aldehyde group and a hydroxy group, and
when RG is a group containing a naphthalene ring and a plurality of R¹ is present, R¹ contains no combination of a hydroxy group and a carboxyl group.

### Aspect 29

The compound according to any one of the aspects 1 and 24 to 28, wherein
R¹ is selected from —OR², —COOR³, —CH₂—OR⁴, or —CHO,
wherein
R² is a hydrogen atom, an alkyl group having 1 to 30 carbon atoms and optionally having a substituent, or an aryl group having 1 to 30 carbon atoms and optionally having a substituent;
R³ is a hydrogen atom, an alkyl group having 1 to 29 carbon atoms and optionally having a substituent, or an aryl group having 1 to 29 carbon atoms and optionally having a substituent; and
R⁴ is a hydrogen atom, an alkyl group having 1 to 29 carbon atoms and optionally having a substituent, or an aryl group having 1 to 29 carbon atoms and optionally having a substituent.

### Aspect 30

The compound according to any one of the aspects 1 and 24 to 29, wherein R¹ has a protective group.

### Aspect 31

The compound according to the aspect 1, wherein RG is a group derived from benzene optionally having a substituent, naphthalene optionally having a substituent, anthracene optionally having a substituent, phenanthrene optionally having a substituent, pyrene optionally having a substituent, fluorene optionally having a substituent, or adamantane optionally having a substituent.

### Aspect 32

The compound according to the aspect 31, wherein RG is a group derived from benzene optionally having a substituent, naphthalene optionally having a substituent, or adamantane optionally having a substituent.

### Aspect 33

The compound according to the aspect 32, wherein RG is a group derived from benzene optionally having a substituent.

### Aspect 34

The compound according to the aspect 32, wherein RG is a group derived from naphthalene optionally having a substituent.

### Aspect 35

The compound according to the aspect 32, wherein RG is a group derived from adamantane optionally having a substituent.

### Aspect 36

The compound according to any one of the aspects 1 and 24 to 35, wherein the compound is represented by the formula (Bz), the formula (N), or the formula (Ad), which will be mentioned later.

### Aspect 37

The compound according to the aspect 36, wherein the compound is represented by any one of the formulas (Bz1), (n), and (Ad1), which will be mentioned later.

### Aspect 38

The compound according to the aspect 37, wherein the compound is represented by any one of the formulas (Bz1-1), (n), (2n), (3n), (1a), and (2a), which will be mentioned later.

### Aspect 39

The compound according to the aspect 33, wherein the compound is represented by any one of the formulas (1b), (Bz1-2-1), (Bz2), (1b-1), (Bz1-3-1), and (Bz3), which will be mentioned later.

### Aspect 40

The compound according to the aspect 34, wherein the compound is represented by any one of the formulas (n), (2n), and (3n), which will be mentioned later.

### Aspect 41

The compound according to the aspect 40, wherein the compound is represented by any one of the formulas (1n′), (2n-1), (3n-1), and (3n-2), which will be mentioned later.

### Aspect 42

The compound according to the aspect 35, wherein the compound is represented by any one of the formulas (1a) and (2a), which will be mentioned later.

### Aspect 43

The compound according to any one of the aspects 1, 24 to 35, and 36, wherein R¹ is a hydroxy group, a carboxyl group, an ester group, or a hydroxyalkyl group; and
when A is A′ represented by —O—R^{a}—O—R^{b}, the compound contains one or more A′.

### Aspect 44

The compound according to the aspect 43, wherein the compound is represented by any one of the formulas (1b-3), (Bz1-2-2), (Bz2-1), (1b-4), (Bz1-3-2), and (Bz3), which will be mentioned later.

### Aspect 45

The compound according to the aspect 34, 40, or 41, wherein R¹ is a hydroxy group, a carboxyl group, an ester group, or a hydroxyalkyl group.

### Aspect 46

The compound according to the aspect 34, 40, 41, or 45, wherein the compound is represented by any one of the formulas (1n), (1n′-1), (2n-1-1), (3n-1-1), and (3n-2-1), which will be mentioned later.

### Aspect 47

The compound according to the aspect 35 or 42, wherein R₁ is a hydroxy group, a carboxyl group, an ester group, or a hydroxyalkyl group.

### Aspect 48

The compound according to the aspect 35, 42, or 47, wherein the compound is represented by any one of the formulas (1a-1) and (2a-1), which will be mentioned later.

### Aspect 49

The compound according to any one of the aspects 1 and 24 to 48, wherein the compound expresses a sensitizing effect in radiation irradiation of a composition for lithography.

### Aspect 50

The compound according to any one of the aspects 1 and 24 to 48, wherein the compound is used for lithography.

### Advantageous Effects of Invention

A compound useful as a lithography composition can be provided. Further, a sensitizing effect can be obtained by using the compound and composition of the present invention in lithography process.

### DESCRIPTION OF EMBODIMENTS

Hereinafter, the present invention will be described in detail. In the present invention, “X to Y” includes X and Y that are end values thereof.

### 1. Compound

The compound according to the present embodiment is represented by the following formula (1).

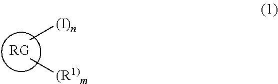

### [RG]

In the formula, RG is a group containing at least one cyclic structure. The valence of RG is arbitrarily regulated depending on the number of substituents other than I, R¹, and R¹, which will be mentioned later, and the like. The group containing a cyclic structure is only required to contain an aromatic ring, an alicyclic ring, or a heterocyclic ring, and is preferably a group having 6 to 60 carbon atoms, and more preferably a group derived from any of the following compounds:
aromatic rings such as benzene, naphthalene, biphenyl, anthracene, phenanthrene, pyrene, or fluorene, and alicyclic rings such as cyclohexane, cyclododecane, dicyclopentane, tricyclodecane, or adamantane. RG may not contain a ring aggregation in which monocycles are bonded by a single bond (e.g., biphenyl, binaphthyl, or bicyclopropyl). In this case, specifically, RG is preferably a group having at least one cyclic structure selected from a monocyclic aromatic ring structure, a fused ring aromatic structure, and a polycyclic alicyclic structure.

Among them, from the viewpoint of easy availability and the like, RG is preferably a group derived from benzene, naphthalene, phenanthrene, fluorene, or adamantane.

### [I]

In the formula, I is an iodine atom. n represents the number of I, and is an integer of 1 to 5. From the viewpoint of the sensitizing effect, the solubility in a solvent, and chemical stability, n is preferably an integer of 1 to 3, and more preferably 1 or 2. By making n larger than 1, the sensitizing effect can be obtained, and by setting n to 5 or less, the solubility of the compound in a solvent component that is generally used for semiconductors and the stability of the compound itself can be ensured.

### [R¹]

R¹ is a monovalent functional group having 0 to 30 carbon atoms, containing no polymerizable unsaturated bond, and being optionally the same or different. When R¹ is converted into another group or bonded to another group, a derivative of the compound of the formula (1) can be produced. The polymerizable unsaturated bond refers to an ethylenic double bond or triple bond. When R¹ is as described above, stability and solubility are excellent.

R¹ is not an alkyl group, but is a functional group. R¹ is, for example, an alkoxy group having 1 to 30 carbon atoms, a carboxyl group having 1 to 30 carbon atoms, a carboxylate group having 2 to 10 carbon atoms, an alkoxyalkyl group having 2 to 30 carbon atoms, or a hydroxyalkyl group, an aldehyde group, a halogen atom other than iodine, a nitro group, an amino group, a thiol group, or a hydroxy group. Among them, from the viewpoint of the sensitizing effect and the like, R¹ is preferably a hydroxy group, a carboxyl group, an ester group, or a hydroxyalkyl group. Among these groups, the group capable of having a substituent optionally has a substituent. Unless otherwise defined, the term “substituted” means that one or more hydrogen atoms in a functional group are substituted with a substituent. Examples of the “substituent” include, but are not particularly limited to, a halogen atom, a hydroxy group, a cyano group, a nitro group, a thiol group, a heterocyclic group, a linear aliphatic hydrocarbon group having 1 to 20 carbon atoms, a branched aliphatic hydrocarbon group having 3 to 20 carbon atoms, a cyclic aliphatic hydrocarbon group having 3 to 20 carbon atoms, an aryl group having 6 to 20 carbon atoms, an alkoxyl group having 1 to 20 carbon atoms, an amino group having 0 to 20 carbon atoms, an alkenyl group having 2 to 20 carbon atoms, an alkynyl group having 2 to 20 carbon atoms, an acyl group having 1 to 30 carbon atoms (preferably an alkyloyloxy group having 1 to 20 carbon atoms or an aryloyloxy group having 7 to 30 carbon atoms), an alkoxycarbonyl group having 2 to 20 carbon atoms, or an alkylsilyl group having 1 to 20 carbon atoms. These groups may form a ring structure in the substituent, or with a group having a substituent or another R¹. Suitable examples of the group that may form a ring structure include a glycidyl group, a cyclic acetal group, and a group that forms an acetal protective group structure by two adjacent hydroxy groups.

Among them, R¹ is preferably selected from a group represented by —OR², an alkoxy group having 1 to 30 carbon atoms, a hydroxy group, a carboxyl group having 1 to 30 carbon atoms, a carboxylate group having 2 to 10 carbon atoms, an alkoxyalkyl group having 2 to 30 carbon atoms, an alkoxyalkyl group having 2 to 30 carbon atoms, a hydroxyalkyl group having 1 to 10 carbon atoms, or an aldehyde group. Herein, R² is a hydrogen atom, an alkyl group having 1 to 30 carbon atoms, an aryl group having 1 to 30 carbon atoms, or a cyclic alkyl ether group having 1 to 5 carbon atoms. The carboxyl group or carboxylate group is more preferably represented by —COOR³. Herein, R³ is a hydrogen atom, an alkyl group having 1 to 29 carbon atoms, or an aryl group having 1 to 29 carbon atoms. The alkoxyalkyl group or hydroxyalkyl group is more preferably represented by —CH₂—OR⁴. Herein, R⁴ is a hydrogen atom, an alkyl group having 1 to 29 carbon atoms, or an aryl group having 1 to 29 carbon atoms. The alkyl group or aryl group optionally has a substituent. Examples of the substituent include an alkoxy group. Thus, in one aspect, R² in —OR² may be —CH₂—OC₂H₅.

The alkyl group in R² to R⁴ preferably contains a methyl group, an ethyl group, or a propyl group (including isomers, the same applies hereinafter). The aryl group is preferably a phenyl group or a naphthyl group.

R¹ may have a protective group. The protective group refers to a group that dissociates under specific conditions, and is also referred to as the dissociable group. The protective group is preferably an acid dissociable group which dissociates in the presence of an acid. Preferable examples of the group include a 1-substituted ethyl group, a 1-substituted-n-propyl group, a 1-branched alkyl group, a silyl group, an acyl group, a 1-substituted alkoxymethyl group, a cyclic ether group, an alkoxycarbonyl group, or an alkoxycarbonylalkyl group. In one aspect, R¹ may be a hydroxy group or carboxyl group protected by a protective group. For example, R¹ is —O—CH₂—O—R′. R′ is, for example, an alkyl group having 1 to 5 carbon atoms. This aspect corresponding to the case where R¹ is —OR² (provided that R² is CH₃) and R² has an alkoxy group (—O—R′) as a substituent. When R¹ is a group having a protective group, R′ is sometimes denoted as A or A′ as mentioned later.

In the formula, m represents the number of R¹, and is an integer of 1 to 5. From the viewpoint of the solubility in a solvent and the like, m is preferably 4, 3, 2, or 1. When m is 2 or 3, a plurality of R¹ may be different or the same. m is more preferably 2 or 3, and further preferably 2. The sum of m and n is arbitrarily regulated depending on the valence of RG.

The compound may have an organic group other than R¹ as a substituent, if required. Examples of the organic group include an alkyl group having 1 to 30 carbon atoms. A plurality of the groups may be present. However, the compound preferably contains no organic group other than R¹ and iodine.

When RG is a group containing a benzene ring and a plurality of R¹ is present, R¹ contains no combination of an alkoxy group and an aldehyde group, no combination of an alkoxy group and a hydroxy group, and no combination of an aldehyde group and a hydroxy group. The alkoxy group having a protective group is excluded herein. The alkoxy group is, for example, a methoxy group or an ethoxy group. When RG is a group containing a naphthalene ring and a plurality of R¹ is present, R¹ contains no combination of a hydroxy group and a carboxyl group.

When RG is a group derived from benzene, naphthalene, anthracene, pyrene, a heteroaromatic compound, or a polycyclic alicyclic compound, R¹ is preferably composed of one or more R^{f} and zero or more R^{g}. R¹ is composed of one or more R^{f′} and zero or more R^{g}. R^{f} is a hydroxy group and an ether group having a protective group. R^{f′} is a hydroxy group, or an ether group having a protective group eliminatable by acid, alkali, or heat. R^{g} is a hydrocarbon group having 0 to 30 carbon atoms and optionally containing a substituent. In particular, when RG has a benzene or naphthalene structure, R¹ is preferably composed of one or more R^{f} selected from the group consisting of a hydroxy group and an ether group having a protective group, and zero or more hydrocarbon groups R^{g} having 0 to 30 carbon atoms and optionally containing a substituent. When the compound of the formula (1) has these groups as R¹, the reaction for linking the compound to other compounds can be smoothly progressed.

As mentioned above, the compound of the formula (1) can be linked to other compounds. For example, the compound of the formula (1) can be formed into a dimer to a pentamer. The polymer will be mentioned later.

### 1-2. Preferable Aspect

### (1) First Aspect

In the first aspect, RG is a benzene ring.

In the present aspect, the compound represented by the formula (1) (hereinafter, referred to as the “compound of the formula (1)” or the like) is preferably represented by the formula (Bz), from the viewpoint of the sensitizing effect, easy availability, and the like.

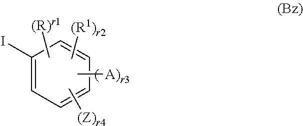

In the formula, I and R¹ are defined as mentioned above. From the viewpoint of the sensitizing effect, R¹ is preferably a hydroxy group, a carboxyl group, an ester group, or a hydroxyalkyl group.

A is a group having a protective group. A becomes a functional group by removing the protective group, and thus, A is one of R¹. As mentioned above, the protective group is preferably an acid dissociable group. Thus, the group having a protective group is preferably a group in which a hydroxy group or a carboxyl group is protected by an acid dissociable group. A may be A′ represented by —O—R^{a}—O—R^{b}, and in this case, the compound of the formula (Bz) preferably contains one or more A′. R^{a} and R^{b} will be mentioned later.

R is a hydrogen atom or an organic group other than a functional group. Examples of the organic group include an alkyl group having 1 to 30 carbon atoms.

Z is I, R¹, H, or a linking group for forming a dimer. When Z is a linking group for forming a dimer, two molecules are bonded by a single bond to produce a dimer. The dimer is contained in the compound represented by the formula (DM1a), which will be mentioned later. Z may not contain the linking group for forming a dimer. When Z contains no linking group for forming a dimer, in particular, Z is denoted as Z′.

R¹, R, and A are each bonded to any bondable position. r1 to r4 each independently represent an integer of 0 to 5, and the sum of them satisfies the valence of the benzene ring. Provided that, at least one of r2 and r3 is 1 or more. r1 to r4 are each independently more preferably 1 to 4, further preferably 1 to 3, and particularly preferably 1 or 2. Hereinafter, the preferred aspect of the compound will be described, from the viewpoint of the sensitizing effect, easy availability, and the like.

### [Bz1 System]

The compound of the formula (Bz) is preferably represented by the formula (Bz1). The compound of the formula (Bz1) has one R¹ that is not derived from Z. In the present disclosure, unless otherwise indicated, each substituent of a compound is defined as in the compound group to which the compound belongs.

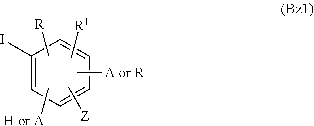

### (Bz1-1 System)

The compound of the formula (Bz1) is preferably represented by the formula (Bz1-1). The compound of the formula (Bz1-1) has one R¹ that is not derived from Z, at the meta-position of I.

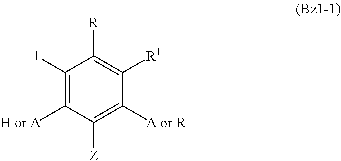

The compound of the formula (Bz1-1) is preferably represented by the formula (1b), and more preferably represented by the formula (1b-3). A and Z, or A and Z′ may form a cyclic structure together with a protective group.

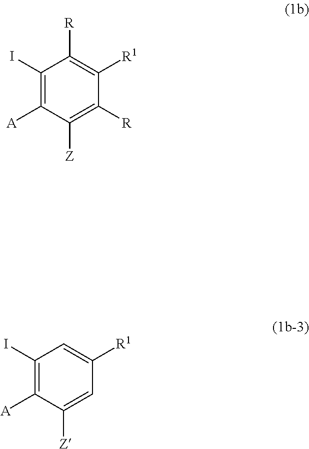

The compound of the formula (Bz1-1) is preferably represented by the formula (1b-1), and more preferably represented by the formula (1b-4).

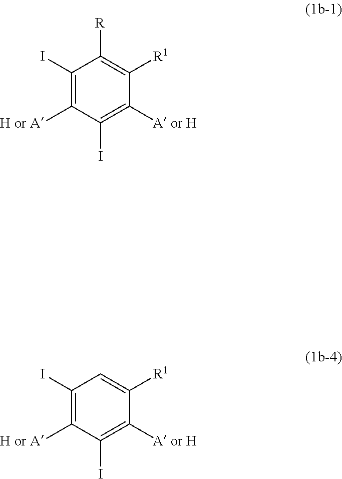

### (Bz1-2 System)

The compound of the formula (Bz1) is preferably represented by the formula (Bz1-2). The compound of the formula (Bz1-2) has one R¹ that is not derived from Z, at the para-position of I. A and Z may form a cyclic structure together with a protective group. In addition, Z and R¹ may form a cyclic structure together with a protective group.

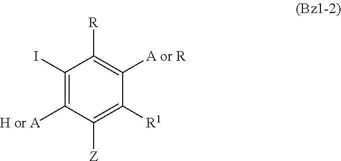

The compound of the formula (Bz1-2) is preferably represented by the formula (Bz1-2-1), and more preferably represented by the formula (Bz1-2-2). A and Z, or A and Z′ may form a cyclic structure together with a protective group.

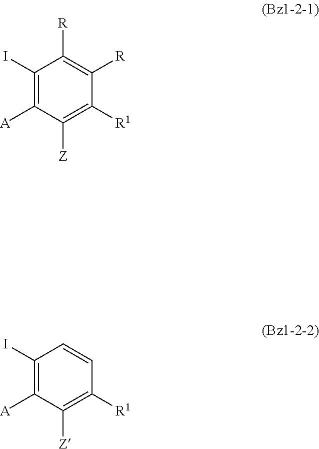

### (Bz1-3 System)

Furthermore, the compound of the formula (Bz1) is preferably represented by the formula (Bz1-3). The compound has one R¹ that is not derived from Z, at the ortho-position of I. A and Z may form a cyclic structure together with a protective group.

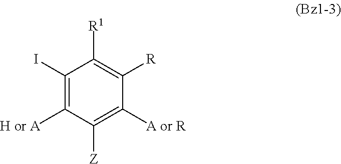

The compound of the formula (Bz1-3) is preferably represented by the formula (Bz1-3-1), and more preferably represented by the formula (Bz1-3-2).

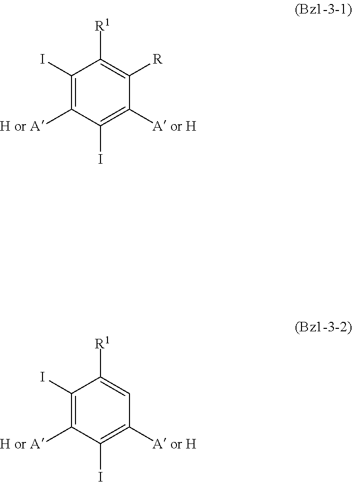

A′ is a group having a protective group, and represented by —O—R^{a}—O—R^{b}, —O—CO—O—R^{b}, —O—R^{a}—CO—O—R^{b}, or —O—R^{a}—O—CO—R^{b}. R^{a} is a linear or branched alkyl group having 1 to 3 carbon atoms. R^{b} is a monovalent linear, branched, or cyclic alkyl group having 1 to 3 carbon atoms or a divalent cyclic alkyl group, and forms a ring with an adjacent oxygen atom. A cyclic structure containing R^{a} and R^{b} may be formed. Provided that, one or more A′ are present.

### [Bz2 System]

The compound of the formula (Bz) is preferably represented by the formula (Bz2). The compound has two R¹ that are not derived from Z at positions not adjacent to each other.

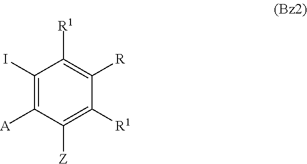

The compound (Bz2) is preferably represented by the formula (Bz2-1).

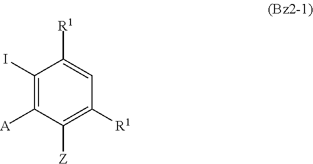

### [Bz3 System]

The compound of the formula (Bz) is preferably represented by the formula (Bz3). The compound has two R¹ that are not derived from Z at positions adjacent to each other. A′ is as defined above, and one or more A′ are present.

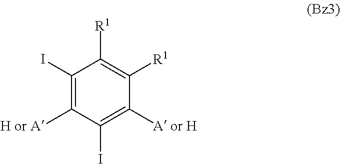

### [Particularly Preferable Aspect]

Among the above, in particular, the compound of the formula (1b-1) is preferable as the compound of the formula (Bz1), from the viewpoint of the sensitizing effect. The compound has R¹, two iodine atoms, and one or more A′. Hereinafter, the compound of the formula (1b-1) will be described.

R¹ is preferably a hydroxyalkyl group or an aldehyde group, and particularly preferably a hydroxyalkyl group. Examples of the method for introducing a hydroxyalkyl group into the benzene nucleus include, but are not limited to, a method for introducing a carboxyl group as R¹ and then reducing the compound. The reduction method can be conducted by a known method.

In the formula (1b-1), A′ is a group having a protective group, and represented by —O—R^{a}—O—R^{b}, —O—CO—O—R^{b}, —O—R^{a}—CO—O—R^{b}, or —O—R^{a}—O—CO—R^{b}. R^{a} is a linear or branched alkyl group having 1 to 3 carbon atoms. R^{b} is a monovalent linear, branched, or cyclic alkyl group having 1 to 3 carbon atoms or a divalent cyclic alkyl group, and forms a ring with an adjacent oxygen atom. A cyclic structure containing R^{a} and R^{b} may be formed. Provided that, one or more A′ are present.

In another aspect, R^{b} is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms. The aliphatic group, the aromatic group, the aliphatic group containing a heteroatom, and the aromatic group containing a heteroatom optionally further have a substituent. As the substituent here, the aforementioned substituents are used, but a linear, branched, or cyclic aliphatic group having 1 to 20 carbon atoms or an aromatic group having 6 to 20 carbon atoms is preferable. Among them, R^{b} is preferably an aliphatic group. The aliphatic group in R^{b} is preferably a branched or cyclic aliphatic group. The number of carbon atoms in the aliphatic group is preferably 1 to 20, more preferably 3 to 10, and further preferably 4 to 8. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

As other R^{b}, groups having the following structures can be used.

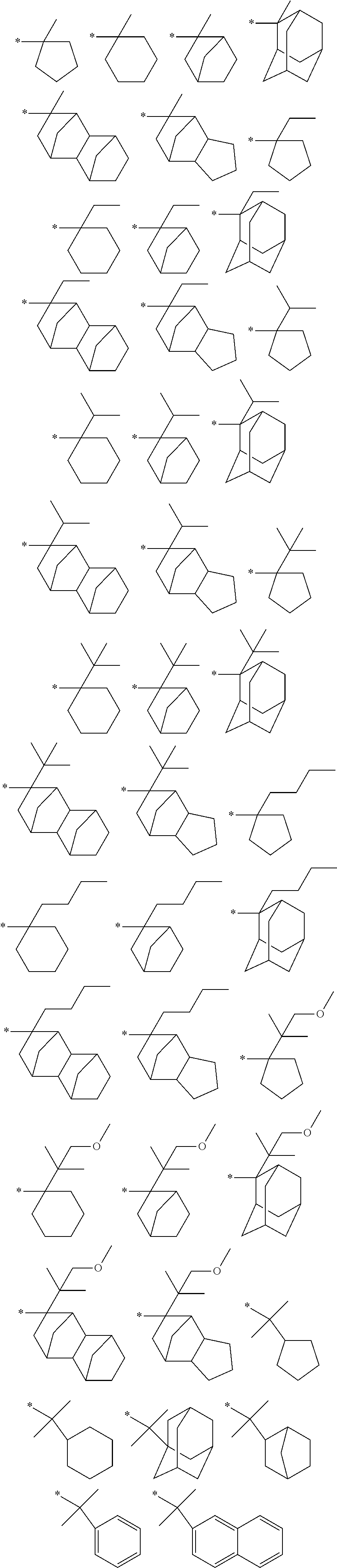

In another aspect, A′ is represented by —CO—O—R^{b} or —C-CyE. CyE is a cyclic ester group optionally having a substituent. A′ is preferably groups represented by the following formulas.

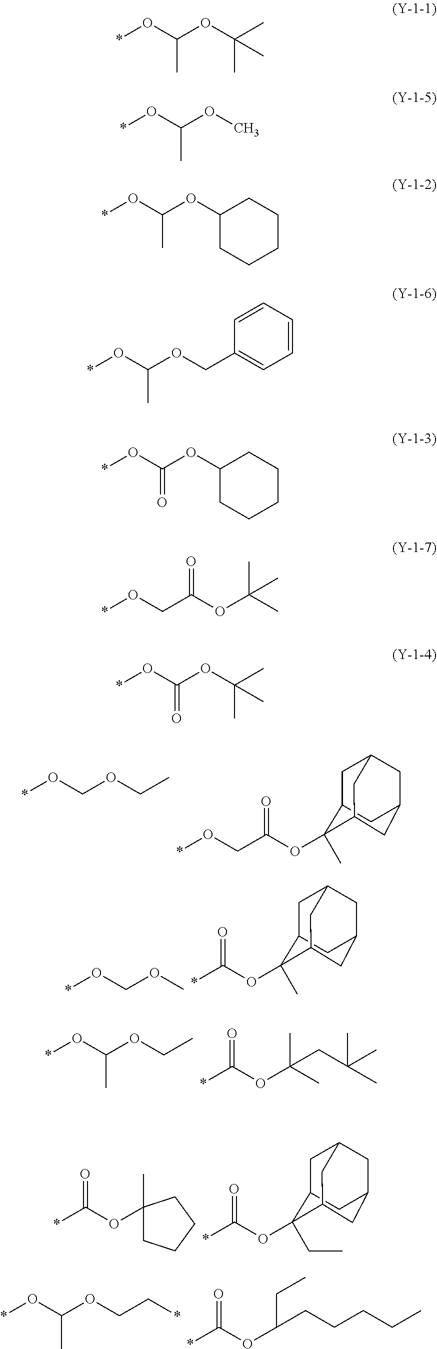

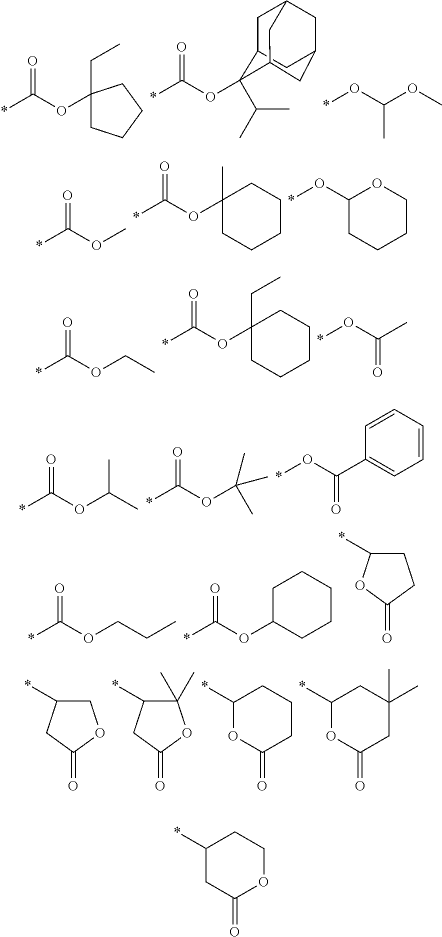

When a plurality of R¹ is present in the compound that belongs to the formula (Bz), R¹ contains no combination of an alkoxy group (excluding one having a protective group) and an aldehyde group, no combination of an alkoxy group (excluding one having a protective group) and a hydroxy group, and no combination of an aldehyde group and a hydroxy group.

In the formula (1b-1), R¹ is preferably a hydroxy group, a carboxyl group, an ester group, an aldehyde group, or a hydroxyalkyl group.

A′ is preferably represented by —O—R^{a}—OR^{b}.

Specific examples of the compound represented by the formula (1b-1) will be listed below, but are not limited thereto.

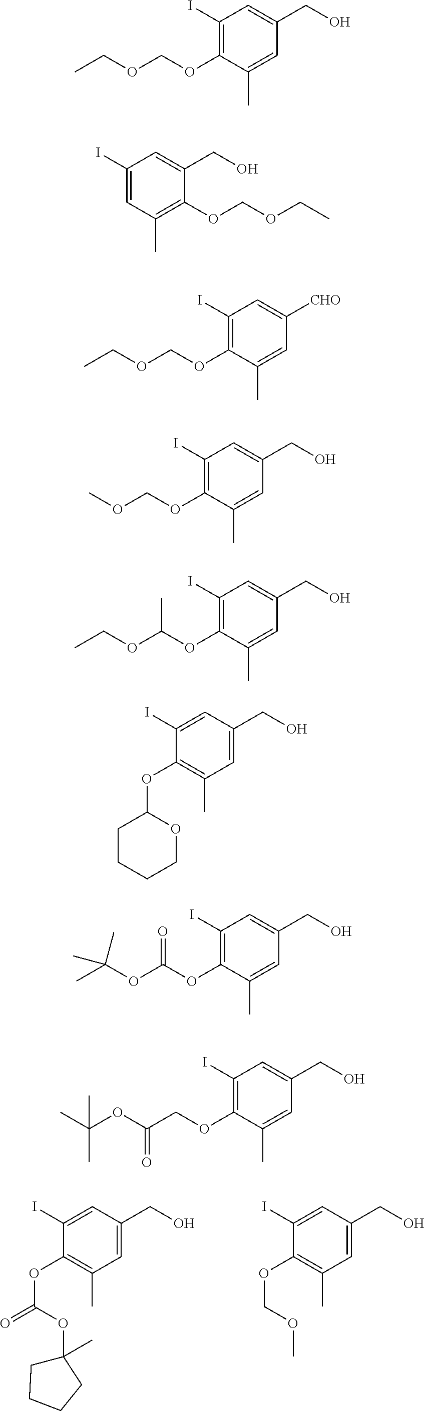

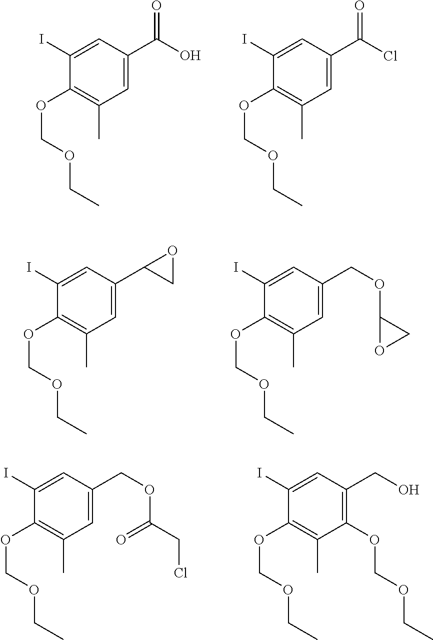

Specific examples of the compound represented by the formula (1b) will be listed below, but are not limited thereto.

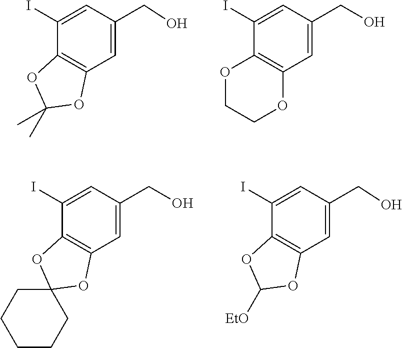 [SMILES: CC1(C)Oc2cc(CO)cc(I)c2O1 ; OCc1cc(I)c2c(c1)OCCO2 ; OCc1cc(I)c2c(c1)OC1(CCCCC1)O2 ; CCOC1Oc2cc(CO)cc(I)c2O1]

Specific examples of the compound represented by the formula (Bz1-3-2) will be listed below, but are not limited thereto.

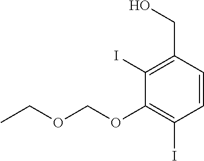

Specific compounds belonging to the compound of the formula (Bz) will be listed below.

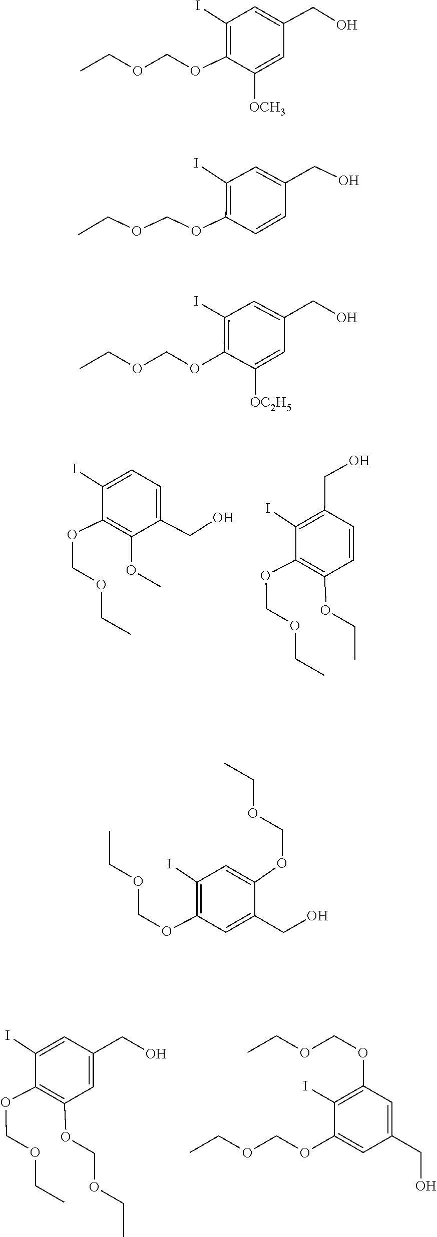

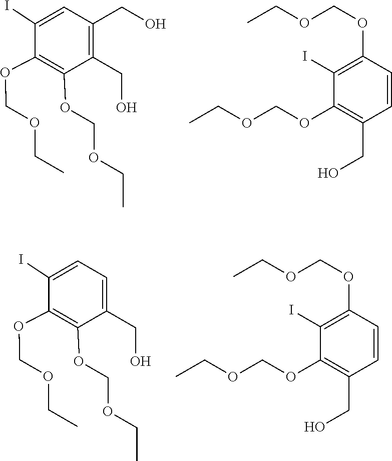

### (2) Second Aspect

In the second aspect, RG is a naphthalene ring. In the second aspect, the compound is preferably represented by the formula (N), from the viewpoint of the sensitizing effect, easy availability, and the like.

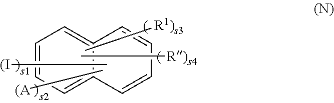

In the formula, R¹ is a monovalent functional group having 0 to 30 carbon atoms, containing no polymerizable unsaturated bond, and being optionally the same or different. R¹ is defined as in the first aspect, and from the viewpoint of the sensitizing effect and the like, R¹ is preferably a hydroxy group, a carboxyl group, an ester group, or a hydroxyalkyl group. A is a group having a protective group, as described in the first aspect. A may be A′ represented by —O—R^{a}—O—R^{b}, and in this case, the compound of the formula (N) preferably contains one or more A′. R″ is a hydrogen atom or an organic group other than R¹. I, R¹, R″, and A are each bonded to any bondable position. s1 is an integer of 1 to 7, s2 to s4 are an integer of 0 to 7, and the sum satisfies the valence of a naphthalene ring. Provided that, at least one of s2 and s3 is 1 or more. s1 is preferably 1 to 5, and more preferably 1 to 3. s2 and s3 are each independently preferably 0 to 5, and more preferably 1 to 3. s4 is preferably 1 to 6. Note that, s4=8−s1−s2−s3 is satisfied. Hereinafter, the preferred compound will be described, from the viewpoint of the sensitizing effect, easy availability, and the like.

In another aspect, the compound in which RG is a naphthalene ring may have a linking group Z for forming a dimer. The compound is represented by the formula (N′). In the formula, each substituent is defined as mentioned above, and any binding position is possible. s1 is an integer of 1 to 7, s2 to s4 are an integer of 0 to 7, s5 is an integer of 1 to 2, and the sum of them satisfies the valence of a naphthalene ring. Provided that, at least one of s2 and s3 is 1 or more.

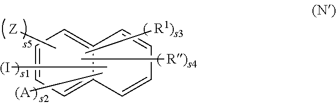

The compound of the formula (N) is preferably represented by the formula (n), the formula (2n), or the formula (3n). R¹, A, and R″ are as defined in the formula (N). x and y are 0 or 1, provided that at least either one is 1. s4′ represents the number of R″ capable of bonding to 1-, 7-, and 8-positions of a naphthalene ring (provided that, the carbon present at the uppermost position of the right ring is set to the 1-position, and the same applies hereinafter), and is an integer of 1 to 3.

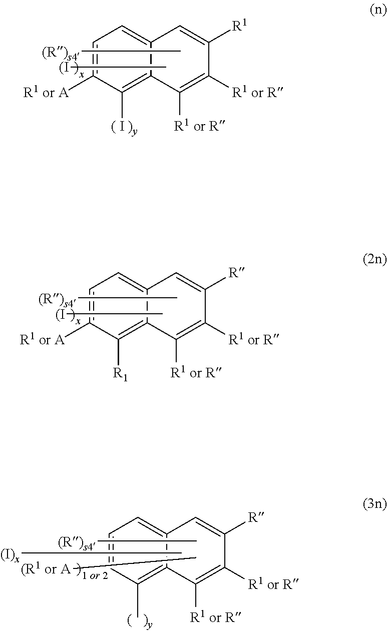

### [(n) System]

The compound of the formula (n) is preferably represented by the formula (1n), and more preferably represented by (1n-1). As mentioned above, when a plurality of R¹ is present, R¹ contains no combination of a hydroxy group and a carboxyl group.

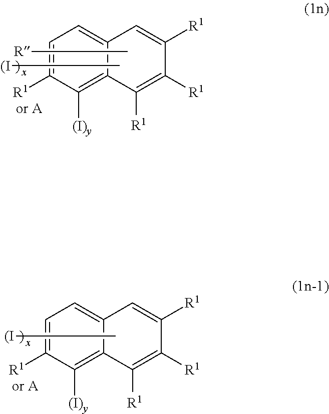

The compound of the formula (n) is preferably represented by the formula (1n′), and more preferably represented by (1n′-1). As mentioned above, when a plurality of R¹ is present, R¹ contains no combination of a hydroxy group and a carboxyl group.

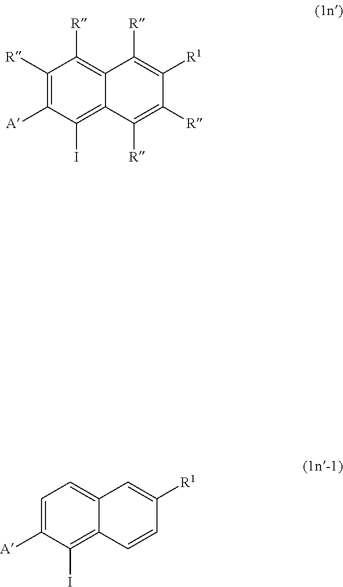

### [(2n) System]

The compound of the formula (2n) is preferably represented by the formula (2n-1), and more preferably represented by (2n-1-1). As mentioned above, when a plurality of R¹ is present, R¹ contains no combination of a hydroxy group and a carboxyl group.

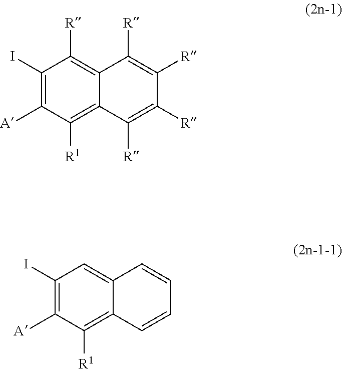

### [(3n) System]

The compound of the formula (3n) is preferably represented by the formula (3n-1), and more preferably represented by (3n-1-1). As mentioned above, when a plurality of R¹ is present, R¹ contains no combination of a hydroxy group and a carboxyl group.

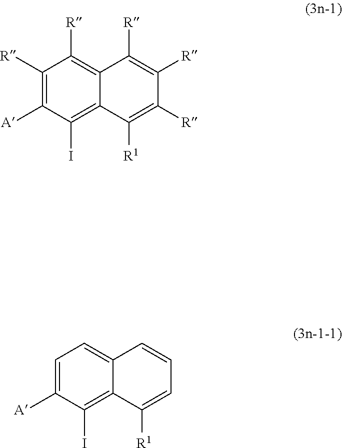

The compound of the formula (3n) is preferably represented by the formula (3n-2), and more preferably represented by the formula (3n-2-1). As mentioned above, when a plurality of R¹ is present, R¹ contains no combination of a hydroxy group and a carboxyl group.

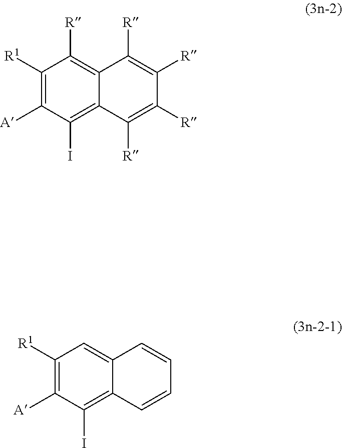

Specific examples of the compound of the formula (N) will be listed below. R^{c} in the following exemplified compounds is a monovalent group having 0 to 29 carbon atoms and containing no polymerizable unsaturated bond.

(1n′)

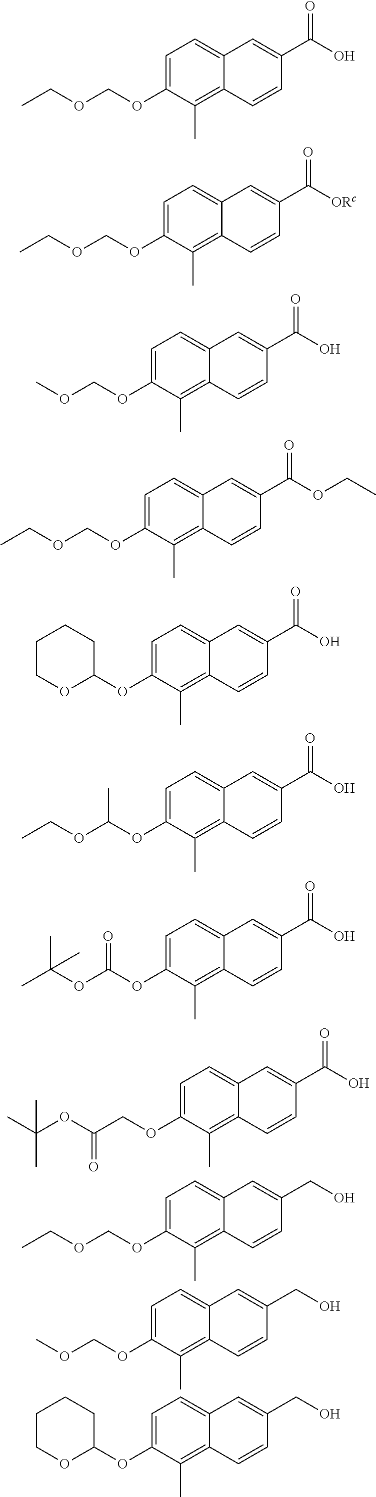

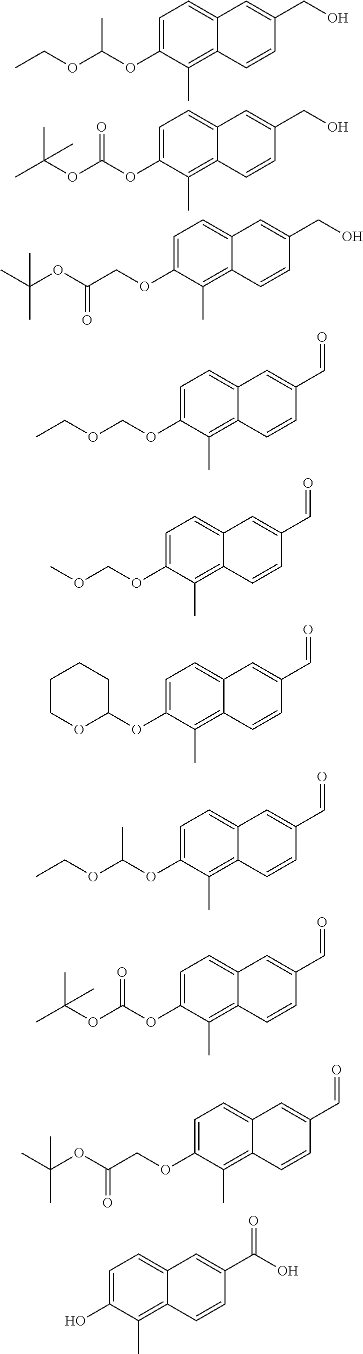

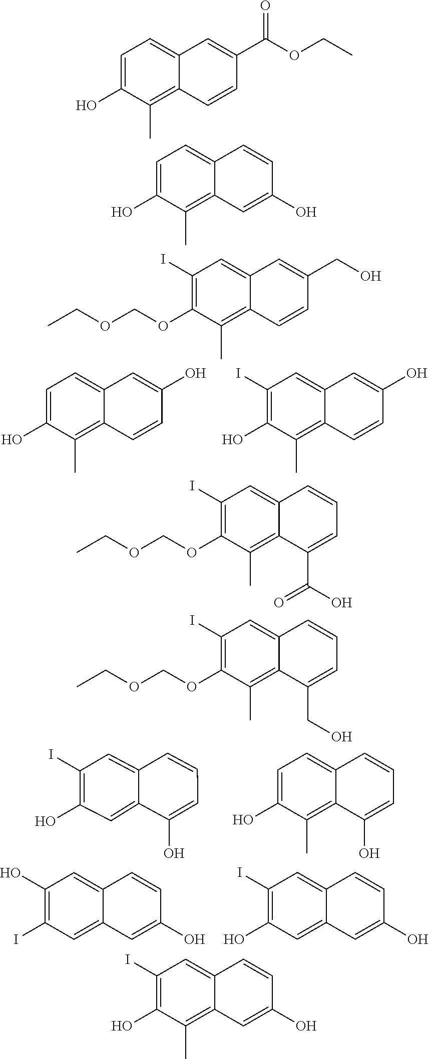

Non-limited specific examples of the compounds belonging to the formula (N) will be disclosed below.

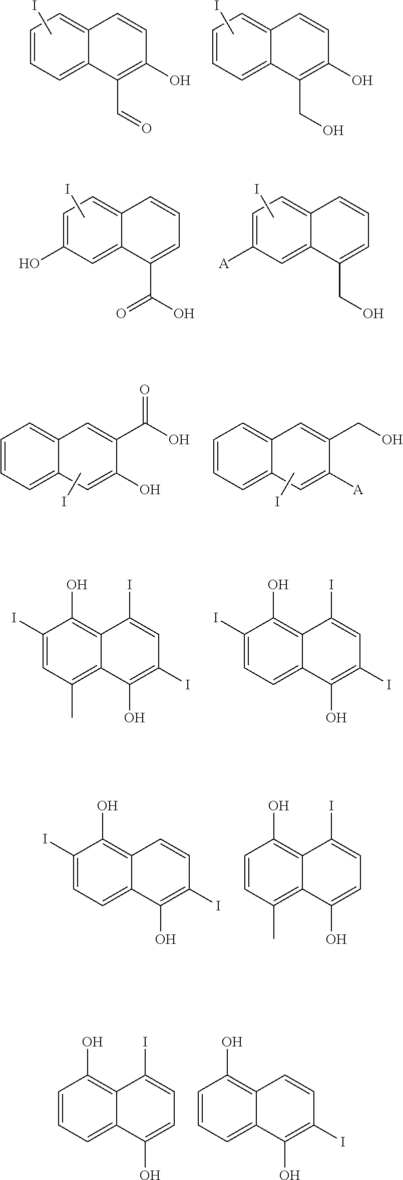

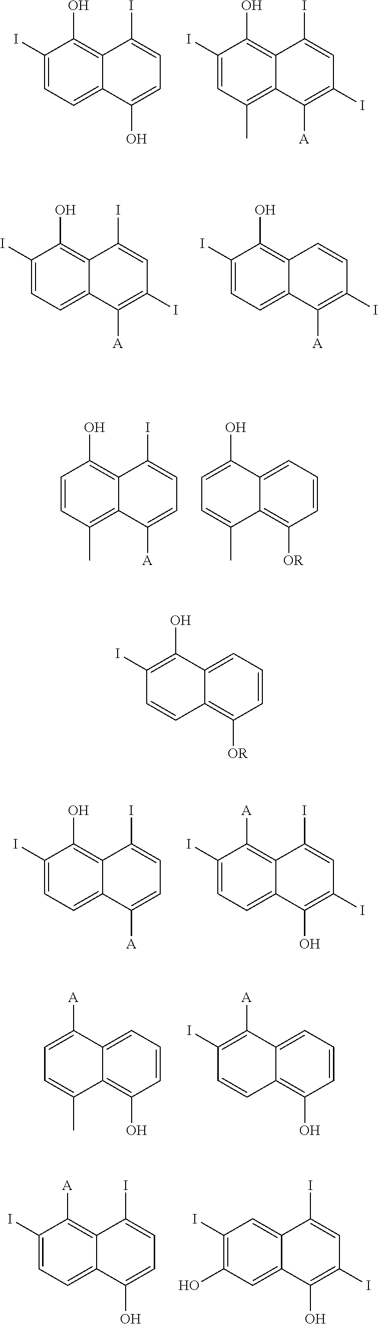

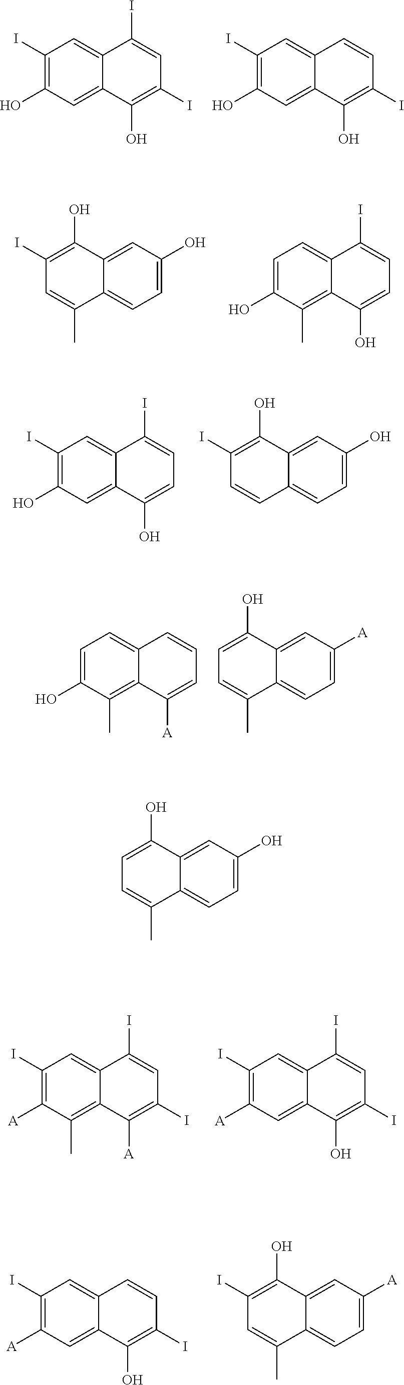

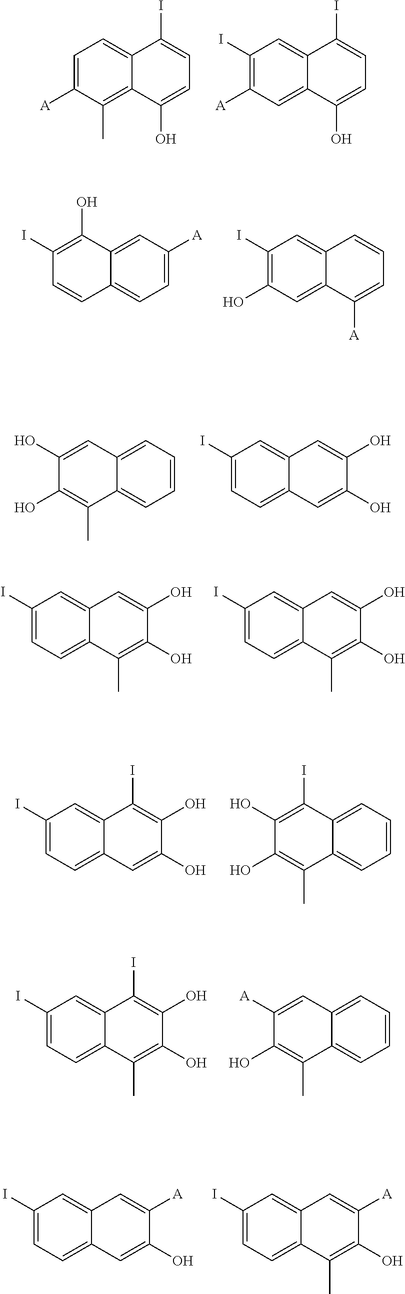

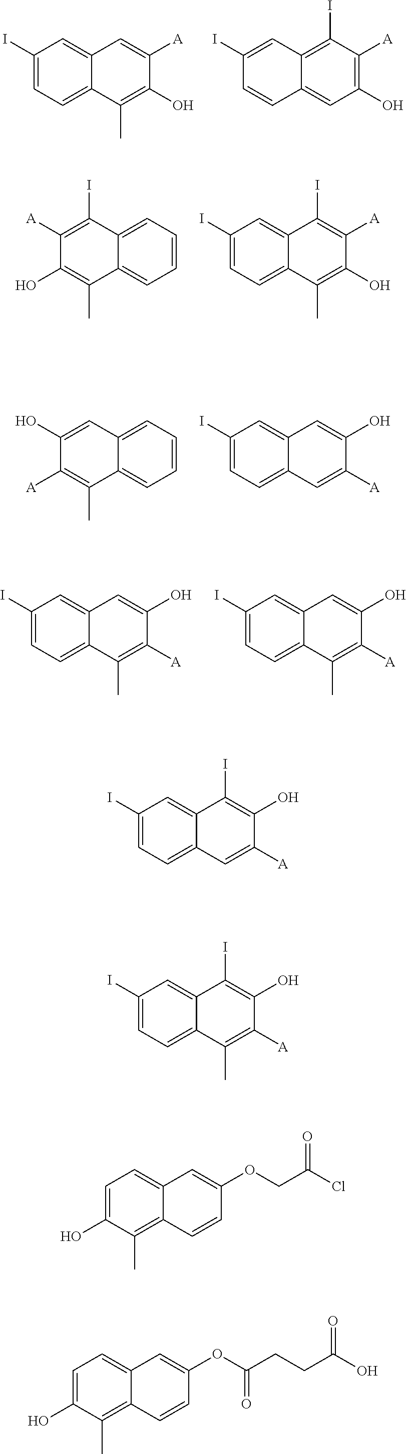

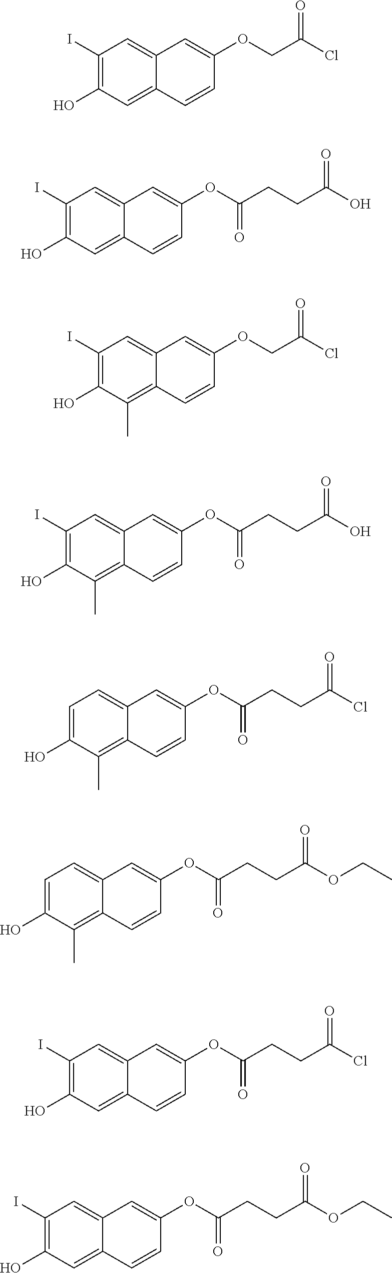

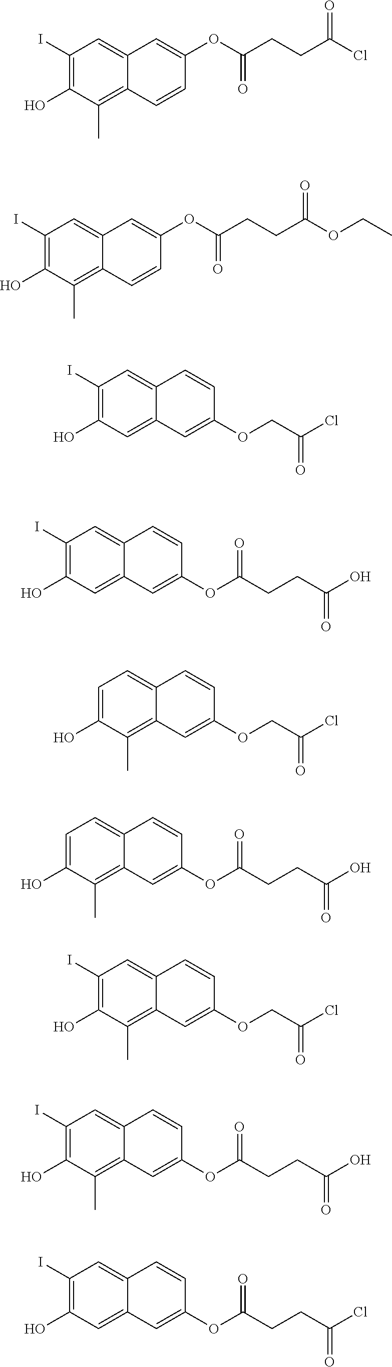

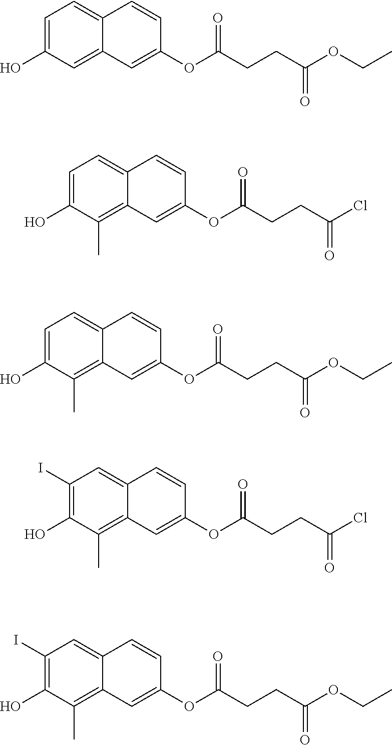 [SMILES: CCOC(=O)CCC(=O)Oc1ccc2ccc(O)cc2c1 ; Cc1c(O)ccc2ccc(OC(=O)CCC(=O)Cl)cc12 ; CCOC(=O)CCC(=O)Oc1ccc2ccc(O)c(C)c2c1 ; Cc1c(O)c(I)cc2ccc(OC(=O)CCC(=O)Cl)cc12 ; CCOC(=O)CCC(=O)Oc1ccc2cc(I)c(O)c(C)c2c1]

In the above formula, A is a group having a protective group. Examples of A include, but are not limited to, the followings.

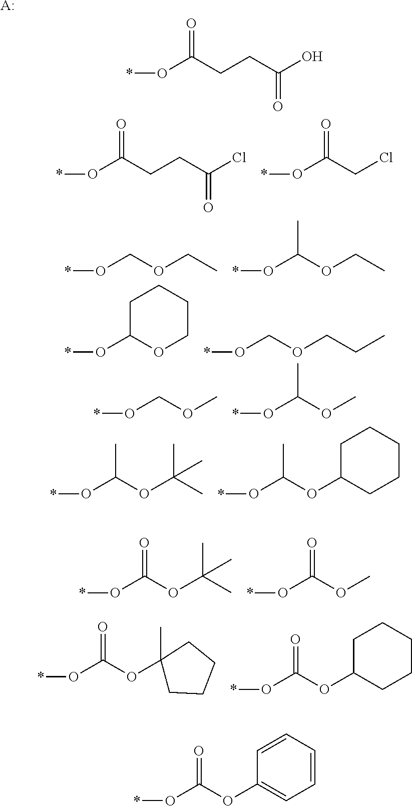

### (3) Third Aspect

In the third aspect, RG is an alicyclic ring having a polycyclic structure having 3 to 30 carbon atoms. The substituents such as I, R¹, and the like in the alicyclic ring may be present at any positions. Specific examples of the alicyclic ring include the following structures. These alicyclic rings may have a further alicyclic structure.

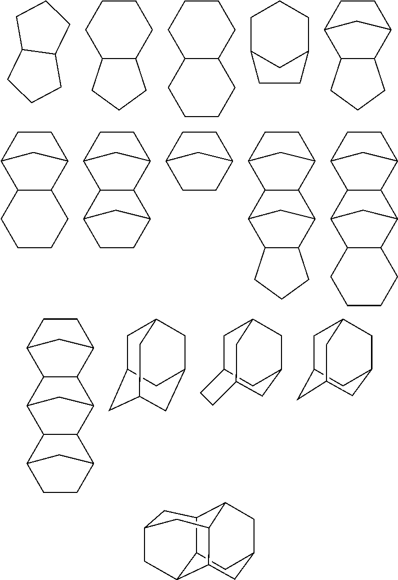

From the viewpoint of the sensitizing effect, easy availability, and the like, RG is preferably an adamantane ring. Thus, in the present aspect, the compound of the formula (1) is preferably represented by the formula (Ad).

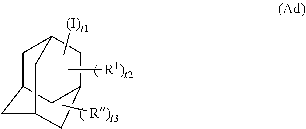

In the formula, I, R¹, and R″ are defined as mentioned above. Provided that, I, R¹, and R″ are each bonded to any position of the adamantane ring. When R¹ is a group having a protective group, the protective group is preferably an acid dissociable group, as mentioned above. Thus, the group having a protective group is preferably a group in which a hydroxy group or a carboxyl group is protected by an acid dissociable group. Among them, from the viewpoint of the sensitizing effect and the like, R¹ is preferably a hydroxy group, a carboxyl group, an ester group, or a hydroxyalkyl group. In this case, another R¹ may be A, or may be A′ represented by —O—R^{a}—O—R^{b}. The compound of the formula (Ad) preferably contains one or more A. t1 is an integer of 1 to 10, t2 is an integer of 1 to 9, t3 is an integer of 0 to 14, and the sum of them satisfies the valence of an adamantane ring. t1 is preferably 1 to 5, and more preferably 1 to 3. t2 is preferably 1 to 5, and more preferably 1 to 3. t3 is preferably 0 to 13, more preferably 5 to 13, and further preferably 8 to 13. From the viewpoint of the sensitizing effect, easy availability, and the like, preferred compounds satisfying t3=16−t1−t2 will be described below. The compound in which RG is an adamantane ring may have a linking group Z for forming a dimer at any position.

The compound of the formula (Ad) is preferably represented by the formula (Ad1). In one aspect, one D is I, and the other D is R¹. In another aspect, two D are R¹.

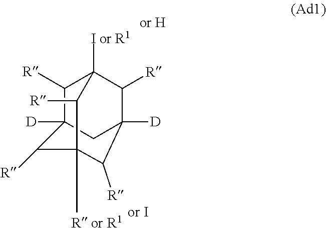

The compound of the formula (Ad1) is preferably represented by the formula (1a), (2a), or (3a).

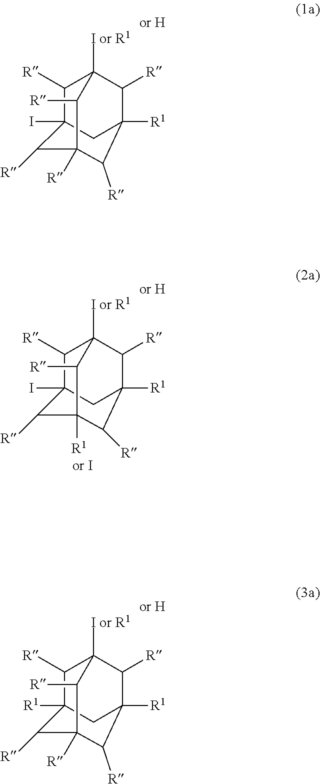

The compounds of the formulas (1a), (2a), and (3a) are preferably represented by the following formulas.

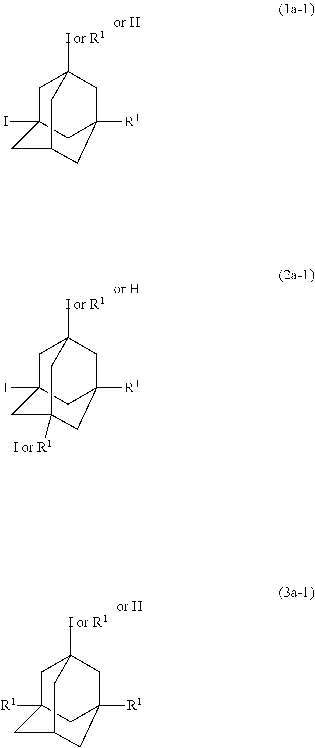

The compound of the formula (Ad1) is preferably represented by the following formulas.

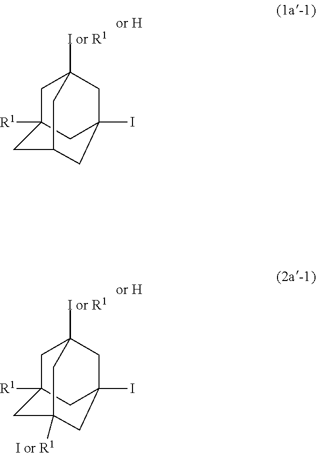

In the formula, I and R¹ are defined as mentioned above. R″ is a hydrogen atom or an organic group other than R¹. The organic group is as mentioned in the first aspect or the second aspect. The compound preferably has one to two I atoms.

In the formulas (1a) to (3a), R¹ is preferably a hydroxy group, a carboxyl group, an ester group (optionally having a substituent such as a halogen other than iodine), or a hydroxyalkyl group.

Non-limited specific examples of the compound represented by the formula (1a-1) will be listed below.

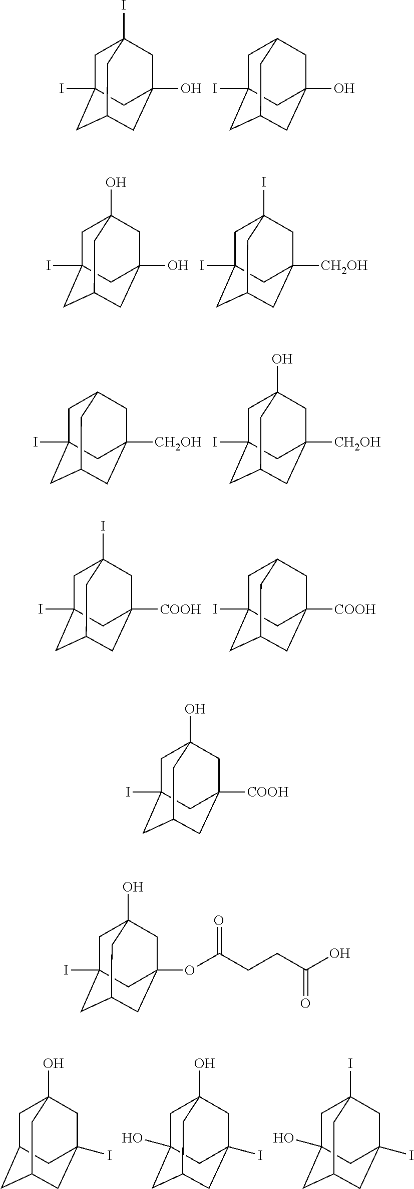

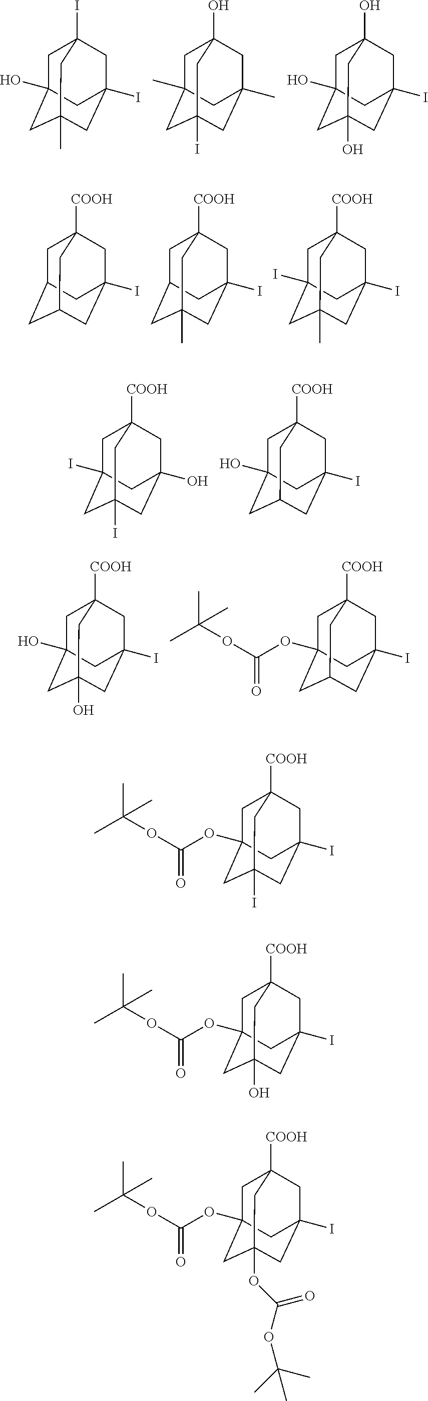

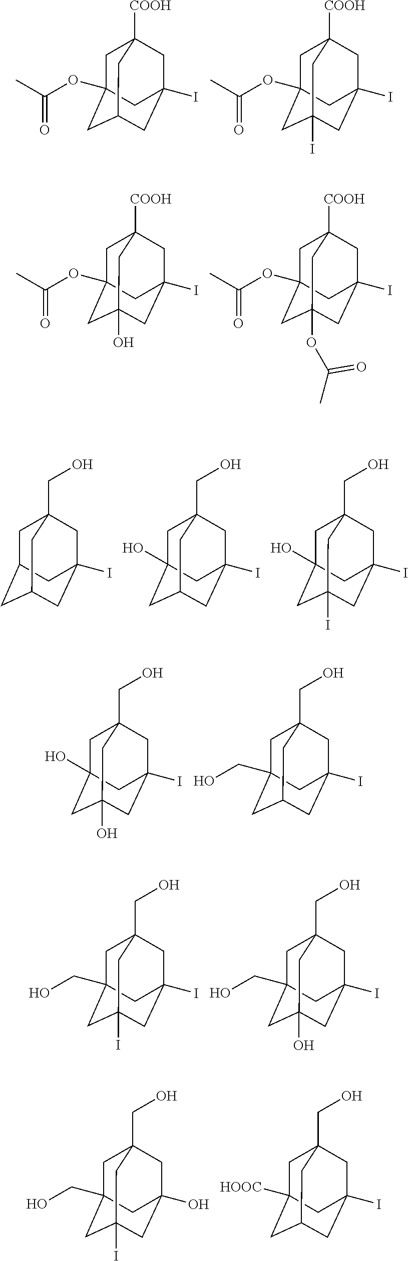

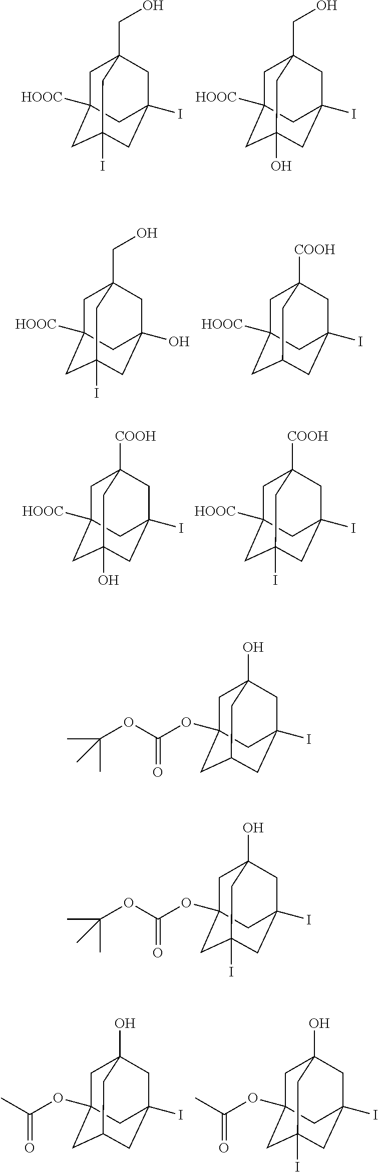

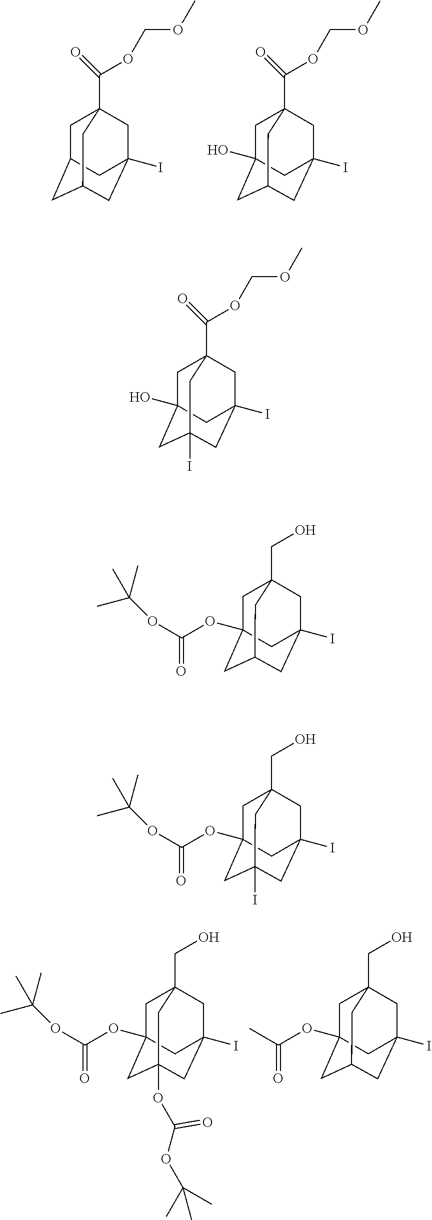

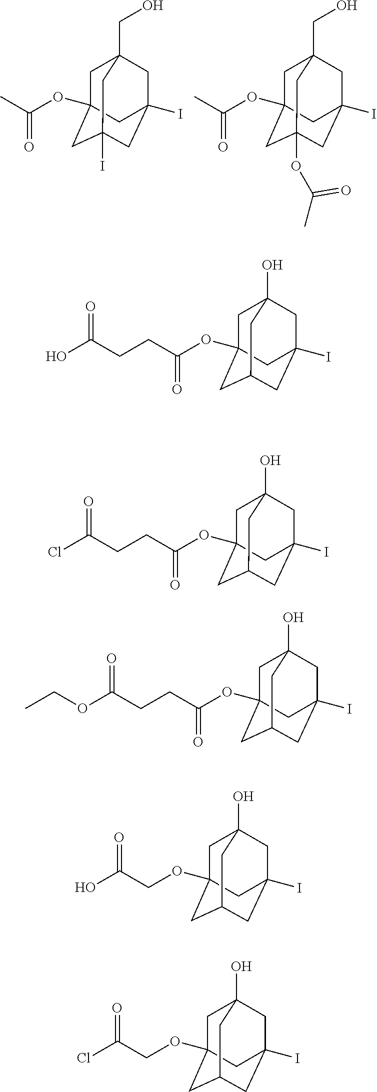

### 1-3. Polymer

As mentioned above, the compound of the formula (1) may be a polymer. In this case, it is preferable that RG contain no ring aggregation in which monocycles are bonded to each other by a single bond (e.g., biphenyl, binaphthyl, or bicyclopropyl). Specifically, RG is preferably a group having at least one cyclic structure selected from a monocyclic aromatic ring structure, a fused ring aromatic structure, and a polycyclic alicyclic structure. In this case, at least some R¹ are preferably the following groups and link two or more molecules: alcohol groups; acetal groups; carboxylate groups; glycidyl groups; carboxyl groups; carboxylic acid halide groups; aldehyde groups; or alkyl groups having 1 to 30 carbon atoms and optionally having a substituent or aryl groups having 1 to 30 carbon atoms and optionally having a substituent, in which the substituent is any one of an alcohol group, an acetal group, a carboxylate group, a glycidyl group, a carboxyl group, and a carboxylic acid halide group.

The carboxylate group may be an alkoxycarbonyloxy group or an aryloxycarbonyloxy group optionally having a substituent.

When the compound of the formula (1) is a polymer, the compound is preferably represented by the following formula.

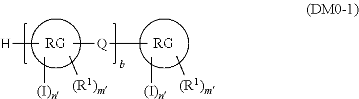

In the formula, RG, I, and R¹ are as defined in the formula (1). n′ is an integer of 1 to 5 and n or less. m′ is an integer of 1 to 5 and m or less. b is an integer of 1 to 4. n′ is preferably 1 to 3. m′ is preferably 1 to 4. b is preferably 1 to 3, and more preferably 1 or 2. Q is a single bond or a group resulting from R¹ that bonds molecules. When Q results from Z, Q is a single bond, and more specifically, it means that the repeat units are bonded by a single bond. When Q results from R¹ that bonds molecules, for example, Q is an ester group.

### [Bz System]

In a preferred aspect, the compound of the formula (DM0-1) is represented by the formula (DM1a).

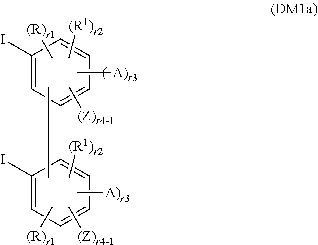

In the formula, R, R¹, A, Z, and r1 to r4 are as defined in the compound of the formula (Bz) system. In the compound, Z is preferably H or R¹.

The compound of the formula (DM1a) is preferably a compound represented by the formula (DM1b).

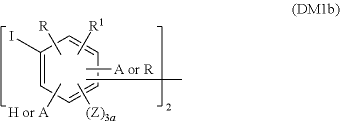

In the formula, I, R, R¹, A, and Z are as defined in the formula (DM1a), and 3a is an integer of 0 to 4, and is preferably 0 or 1.

The compound represented by the formula (DM1b) is preferably a compound represented by the formula (DM1c1).

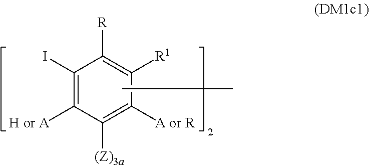

In the formula, I, R, R¹, A, and Z are as defined in the formula (DM1a), and 3a is an integer of 0 to 4, and is preferably 0 or 1.

The compound represented by the formula (DM1c1) is preferably a compound represented by the formula (DM1d11).

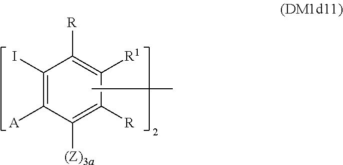

In the formula, I, R, R¹, A, and Z are as defined in the formula (DM1a), and 3a is an integer of 0 to 4, and is preferably 0 or 1.

The compound represented by the formula (DM1c1) is preferably a compound represented by the formula (DM1d12).

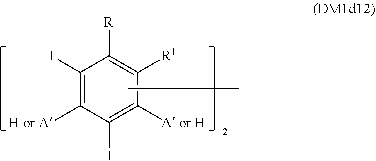

In the formula, I, R, R¹, and Z are as defined in the formula (DM1a). A′ is a group having a protective group, and represented by —O—R^{a}—O—R^{b}, —O—CO—O—R^{b}, —O—R^{a}—CO—O—R^{b}, or —O—R^{a}—O—CO—R^{b}. R^{a} is a linear or branched alkyl group having 1 to 3 carbon atoms. R^{b} is a monovalent linear, branched, or cyclic alkyl group having 1 to 3 carbon atoms or a divalent cyclic alkyl group, and forms a ring with an adjacent oxygen atom. A cyclic structure containing R^{a} and R^{b} may be formed. Provided that, one or more A′ are present.

The compound represented by the formula (DM1b) is preferably a compound represented by the formula (DM1c2).

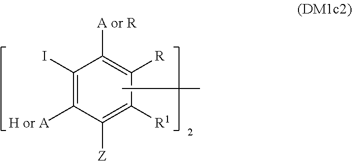

In the formula, I, R, R¹, A, and Z are as defined in the formula (DM1a).

The compound represented by the formula (DM1c2) is preferably a compound represented by the formula (DM1d21) below.

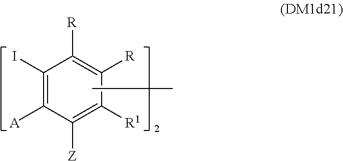

In the formula, I, R, R¹, A, and Z are as defined in the formula (DM1a).

The compound represented by the formula (DM1c1) is preferably a compound represented by the formula (DM1d22).

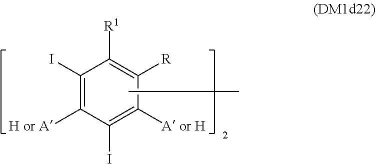

In the formula, I, R, R¹, and Z are as defined in the formula (DM1a). A′ is as defined in the formula (DM1d12).

The compound represented by the formula (DM1b) is preferably a compound represented by the formula (DM1c3).

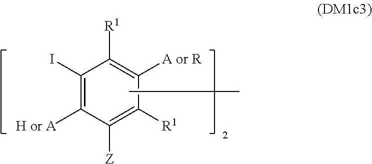

In the formula, I, R, R¹, A, and Z are as defined in the formula (DM1a).

The compound represented by the formula (DM1c3) is preferably a compound represented by the formula (DM1d31).

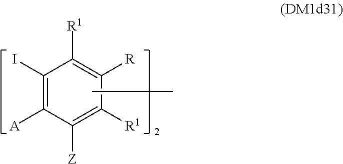

In the formula, I, R, R¹, A, and Z are as defined in the formula (DM1a).

The compound represented by the formula (DM1b) is preferably a compound represented by the formula (DM1c4).

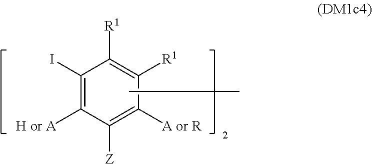

In the formula, I, R, R¹, A, and Z are as defined in the formula (DM1a).

The compound represented by the formula (DM1c4) is preferably a compound represented by the formula (DM1d41).

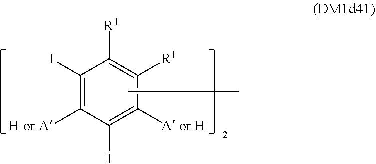

In the formula, I, R, R¹, A, and Z are as defined in the formula (DM1a). A′ is as defined in the formula (DM1d12).

One example of the dimer compound will be shown below. In the formula, I, R, R¹, and A are as defined in the formula (Bz). The compound corresponds to the compound of the formula (1b) in which Z is a linking group for forming a dimer.

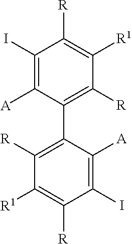

Specific dimer compounds are listed below.

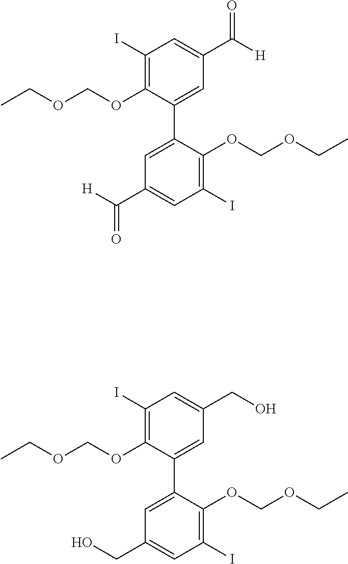

### [N System]

In another preferred aspect, the compound of the formula (DM0-1) is represented by the formula (Dn1).

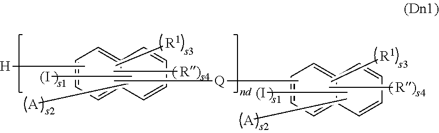

Each substituent and the like are as defined in the formula (N). nd is an integer of 1 to 4. Q is preferably a single bond, and nd is preferably 1.

The compound represented by the formula (Dn1) is preferably a compound represented by the formula (Dn1a).

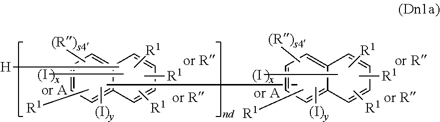

In the formula (Dn1a), I, R¹, R″, A, and nd are as defined in the formula (Dn1). Each of x and y is 0 or 1, and at least either one of x and y is 1. s4′ represents the number of R″ that is bonded to 1-, 7-, and 8-positions of a naphthalene ring.

The compound represented by the formula (Dn1a) is preferably a compound represented by the formula (Dn1b1).

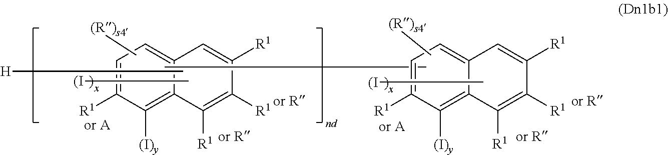

In the formula (Dn1b1), I, R¹, R″, A, and nd are as defined in the formula (Dn1), each of x and y is 0 or 1, and at least either one of x and y is 1. s4′ is as defined in the formula (Dn1a).

The compound represented by the formula (Dn1b1) is preferably a compound represented by the formula (Dn1c11).

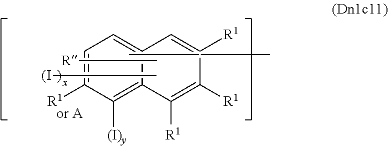

In the formula (Dn1c11), I, R¹, R″, A, and nd are as defined in the formula (Dn1), each of x and y is 0 or 1, and at least either one of x and y is 1. nd is preferably 2.

The compound represented by the formula (Dn1b1) is preferably a compound represented by the formula (Dn1c12).

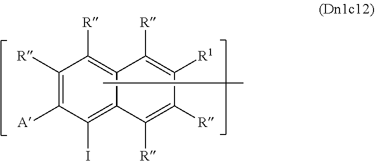

In the formula (Dn1c12), I, R¹, R″, A, and nd are as defined in the formula (Dn1), each of x and y is 0 or 1, and at least either one of x and y is 1. nd is preferably 2.

The compound represented by the formula (Dn1a) is preferably a compound represented by the formula (Dn1b2).

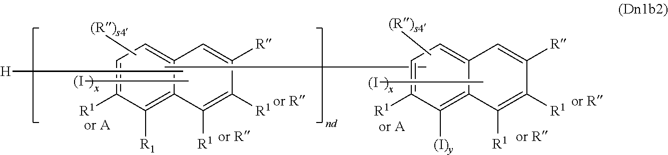

In the formula (Dn1b2), I, R¹, R″, A, and nd are as defined in the formula (Dn1), each of x and y is 0 or 1, and at least either one of x and y is 1. s4′ is as defined in the formula (Dn1a).

The compound represented by the formula (Dn1b2) is preferably a compound represented by the formula (Dn1c21).

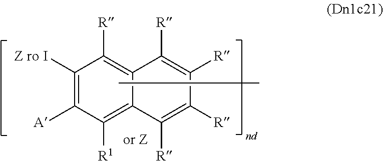

In the formula (Dn1c21), I, R¹, R″, A, and nd are as defined in the formula (Dn1). nd is preferably 2.

The compound represented by the formula (Dn1a) is preferably a compound represented by the formula (Dn1b3).

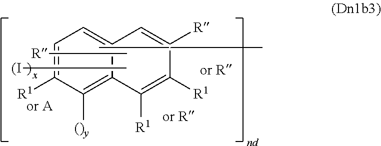

In the formula (Dn1b3), I, R¹, R″, A, and nd are as defined in the formula (Dn1), each y is 0 or 1, and at least either one of x and y is 1. nd is preferably 2.

The compound represented by the formula (Dn1b3) is preferably a compound represented by the formula (Dn1c31).

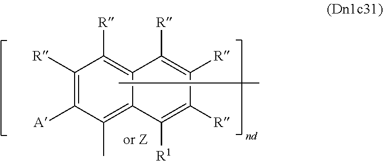

In the formula (Dn1c31), I, R¹, R″, A, and nd are as defined in the formula (Dn1). nd is preferably 2.

The compound represented by the formula (Dn1b3) is preferably a compound represented by the formula (Dn1c32).

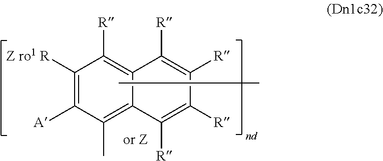

In the formula (Dn1c32), I, R¹, R″, A, and nd are as defined in the formula (Dn1). nd is preferably 2.

Non-limited specific examples of the formula (Dn1) will be listed below.

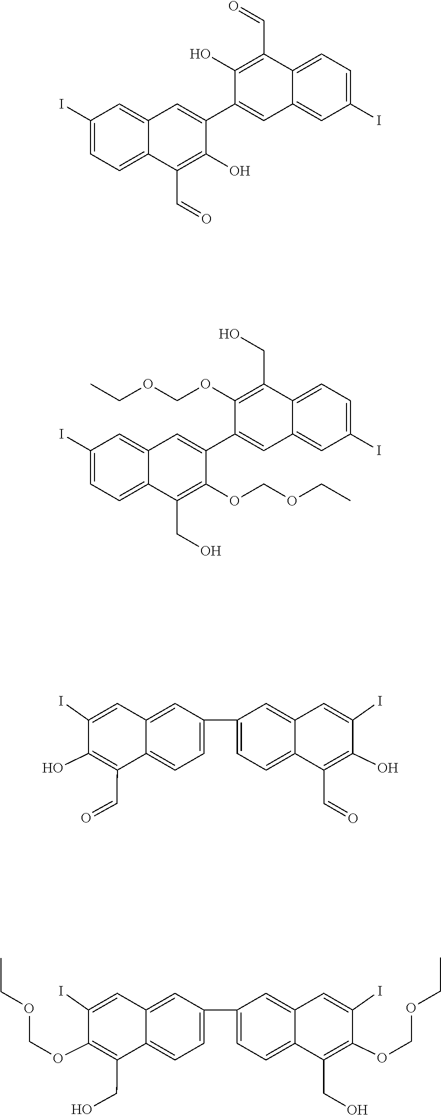 [SMILES: O=Cc1c(O)c(-c2cc3cc(I)ccc3c(C=O)c2O)cc2cc(I)ccc12 ; CCOCOc1c(-c2cc3cc(I)ccc3c(CO)c2OCOCC)cc2cc(I)ccc2c1CO ; O=Cc1c(O)c(I)cc2cc(-c3ccc4c(C=O)c(O)c(I)cc4c3)ccc12 ; CCOCOc1c(I)cc2cc(-c3ccc4c(CO)c(OCOCC)c(I)cc4c3)ccc2c1CO]

### [Ad System]

In still another preferred aspect, the compound of the formula (DM0-1) is represented by the formula (Da1). The compound of the formula (Da1) is more preferably represented by the formula (Da2).

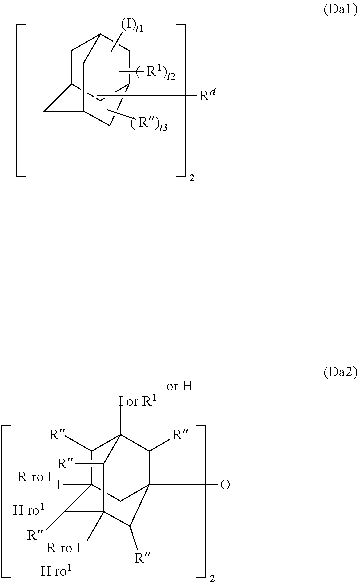

Each substituent and the like are as defined in the formula (Ad).

The compound represented by the formula (Da1) is preferably a compound represented by the formula (Da1a) below.

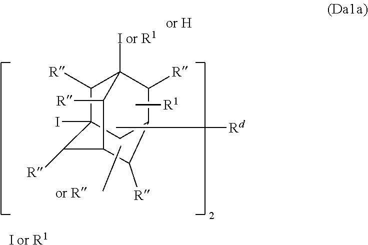

In the formula (Da1a), I, R, R¹, A, Z, and Rd are as defined in the formula (Da1).

The compound represented by the formula (Da1a) is preferably a compound represented by the formula (Da1b).

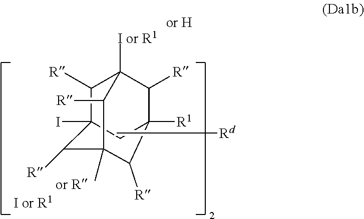

In the formula (Da1b), I, R, R¹, A, and Z are as defined in the formula (Da1a)).

In still another preferred aspect, the compound of the formula (DM0-1) is represented by the formula (Da1c11).

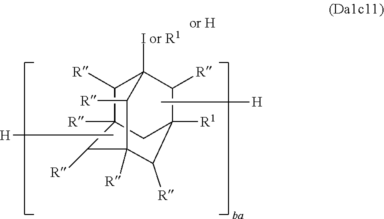

In the formula (Da1c1l), I, R″, and R¹ are as defined in the formula (Da1a), and ba is an integer of 2 to 5).

The compound represented by the formula (Da1b) is preferably a compound represented by the formula (Da1c12).

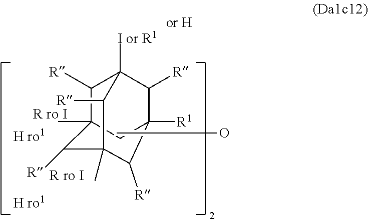

In the formula (Da1c12), I, R, and R¹ are as defined in the formula (Da1a).

### 1-4. Production Method

The compound can be produced by any methods within a range of not impairing the effect thereof. However, a production method comprising a step of introducing an iodine atom or an R¹ group into the compound containing the RG group is preferable. For example, the step of introducing an iodine atom into a compound having an aromatic ring can be conducted by reacting the compound having an aromatic ring with iodine I₂ under alkaline conditions. This reaction enables a compound and a dimer each having a different number of iodine atoms to be produced. The production ratio thereof is regulated by the reaction conditions. In particular, there is a tendency that when the reaction temperature is reduced or the reaction time is reduced, the compound having a small number of iodine atoms increases and the dimer decreases. There is a tendency that when the reaction temperature is increased or the reaction time is increased, the compound having a small number of iodine atoms decreases and the dimer increases. In addition, the step of introducing an iodine atom into a compound having an alicyclic ring can be conducted by reacting the compound having an alicyclic ring with HI (hydrogen iodide). The preferred method for producing the compound comprises an iodination step of introducing an iodine atom, as the substitution reaction, into a raw material that contain RG, a functional group capable of being replaced with an iodine atom by the substitution reaction, and further contains, if required, R¹. In addition, another method for producing the compound can comprise an iodination step of introducing iodine into a raw material that contains RG and, if required, R¹, radically or as a cation or an anion.

### [Iodination Step]

As the iodination step, a method for introducing a halogen from an amino group by a Sandmeyer reaction or the like, a method for reacting iodine chloride in an organic solvent (e.g., Japanese Patent Laid-Open No. 2012-180326, Japanese Patent Laid-Open No. 2000-256231, Japanese Patent Laid-Open No. 2010-159233, and J. Chem. Soc. 636, 1943), a method for dropping iodine in an aqueous alkaline solution of phenol under alkaline conditions in the presence of βcyclodextrin (Japanese Patent Laid-Open No. 63-101342, Japanese Patent Laid-Open No. 2003-64012), or the like can be arbitrarily selected.

Examples of the iodinating agent include, but are not particularly limited to, iodinating agents such as iodine chloride, iodine, and N-iodosuccinimide. The ratio of the iodinating agent to the substrate in the iodination step is preferably 1.2 mol times or more, more preferably 1.5 mol times or more, and further preferably 2.0 mol times or more.

The reaction for introducing iodine can be progressed by at least allowing an iodinating agent to react with the substrate, and the target compound can be obtained according to known reaction conditions for introducing iodine using the methods described in Non Patent Literatures such as Adv. Synth. Catal. 2007, 349, 1159-1172 and Organic Letters; Vol. 6; (2004); p.2785-2788, “Organic Synthesis of Bromine and Iodine Compounds Reagent and Synthesis Method” (supervised by Suzuki, Hitomi, edited by MANAC Inc., Maruzen Publishing Co., Ltd.) and Patent Literatures such as U.S. Pat. Nos. 5,300,506, 5,434,154, US2009/281114, EP1439164, and WO 2006/101318. Examples of the iodinating agent that can be used include, but are not limited to, iodine compounds, monochloride iodine, N-iodosuccinimide, benzyltrimethylammonium dichloroiodate, tetraethylammonium iodide, tetranormalbutylammonium iodide, lithium iodide, sodium iodide, potassium iodide, 1-chloro-2-iodoethane, iodine silver fluoride, tert-butyl hypoiodite, 1,3-diiodo-5,5-dimethylhydantoin, iodine-morpholine complexes, trifluoroacetyl hypoiodite, iodine-iodic acid, iodine-periodic acid, iodine-hydrogen peroxide, 1-iodoheptafluoropropane, triphenylphosphate-methyl iodide, iodine-thallium (I) acetate, 1-chloro-2-iodoethane, and iodine-copper (II) acetate.

One or a plurality of additives can be added in the iodination reaction to promote the reaction and to suppress the by-products. Examples of the additive include an acid such as hydrochloric acid, sulfuric acid, nitric acid, phosphoric acid, acetic acid, p-toluenesulfonic acid, ferric chloride, aluminum chloride, copper chloride, antimony pentachloride, silver sulfate, silver nitrate, and silver trifluoroacetate; a base such as sodium hydroxide, potassium hydroxide, lithium hydroxide, sodium carbonate, potassium carbonate, calcium carbonate, sodium bicarbonate, and potassium bicarbonate; an oxidizing agent such as ammonium cerium (IV) nitrate and sodium peroxodisulfate; an inorganic compound such as sodium chloride, potassium chloride, mercury (II) oxide, and cerium oxide; an organic compound such as acetic anhydride; and a porous material such as zeolite. The ratio of the additive to the iodinating agent in the iodination step is preferably 1.0 mol times, more preferably 1.2 mol times or more, further preferably 1.5 mol times or more, and still more preferably 2.0 mol times or more.

In the iodination step, iodine is preferably introduced into the core by at least using an iodine source and an oxidizing agent. Use of the iodine source and the oxidizing agent is preferable in terms of improving the reaction efficiency and the purity. Examples of the iodine source include the above iodinating agents. Examples of the oxidizing agent include iodic acid, periodic acid, hydrogen peroxide, and other additives (hydrochloric acid, sulfuric acid, nitric acid, p-toluenesulfonic acid, silver trifluoroacetate, and ammonium cerium (IV) nitrate (CAN)). In addition, with respect to phenols having a carboxylic acid group or a nitro group, the iodination reaction can be progressed by using an iodocationic species formed by combining an iodine source such as iodine and silver salt or fuming sulfuric acid. With respect to other relatively inert aromatic compounds, the iodination reaction can be progressed by forming hypoiodous acid and an iodocationic species by combining an iodine source and inorganic salt. As an example of the inorganic salt, potassium peroxodisulphate or the like can be arbitrarily used. A method of introducing iodine into an aliphatic alcohol group by the substitution reaction can also be arbitrarily used. As the iodinating agent, hydrogen halide, phosphorus halide, sulfonyl halide (a combination of NaI/acetone, thionyl halide, the Vilsmeier reagent, or the Appel reaction (a combination of triphenylphosphine and an iodine source) can be arbitrarily used.

Although the reaction in the iodination step can be conducted neat without solvent, examples of the reaction solvent that can be used include a halogenated solvent such as dichloromethane, dichloroethane, chloroform, and carbon tetrachloride; an alkyl solvent such as hexane, cyclohexane, heptane, pentane, and octane; an aromatic hydrocarbon solvent such as benzene and toluene; an alcohol solvent such as methanol, ethanol, 1-propanol, 2-propanol, 1-butanol, and 2-butanol; an ether solvent such as diethyl ether, diisopropylether, and tetrahydrofuran; acetic acid, dimethylformamide, dimethylsulfoxide, and water.

The reaction temperature in the iodination step is not particularly limited, and the temperature may be anywhere from the freezing point to the boiling point of the solvent to be used in the reaction, but is particularly preferably 0° C. to 150° C. To efficiently progress iodination, the reaction system may be refluxed. In addition, to control the concentration of the iodinating agent in the reaction system, the concentration of the iodinating agent in the reaction solution can be controlled by using a reflux tube equipped with Dienstark or the like.

The iodine substitution reaction in the iodination step can be proceeded by at least allowing an iodinating agent to react with the substrate, for example, the intended compound can be obtained by a Sandmeyer reaction using the method described in Chemistry—A European Journal, 24(55), 14622-14626; 2018, Synthesis (2007) (1), 81-84 and the like under known iodine substitution reaction conditions.

### [Protective Group Introduction Step]

The protective group represented by A′ in the preferred method for producing the compound can be introduced into RG by a known method. For example, the method can be arbitrarily selected from the methods described in Green's Protective Groupes in Organic Synthesis (Peter G. M. Wuts, WILEY) p17-p553.

In the compound of the formula (1), when R¹ is a hydroxyalkyl group or an aldehyde group, the compound of the formula (1) can be obtained by, for example, introducing a carboxyl group or an aldehyde group as R¹ and then reducing the compound.

As the reduction method, a known method can be used, and for example, a method using a metal hydride complex compound such as sodium borohydride, lithium aluminum hydride, sodium bis(2-methoxyethoxy)aluminium hydride (SBMEA), or diisobutylaluminum hydride (DIBAL), a method using a metal hydride such as aluminum hydride, or a method using these reducing agents together with a reduction adjuvant such as aluminum chloride or ethanedithiol can be used. The reducing ability of the reducing agent may be regulated by modifying a part of the structure to an alkoxy group or a hydrocarbon group, or by using the reducing agent in combination with a Lewis acid. As the solvent for the reduction reaction, a known solvent such as methanol, ethanol, 2-propanol, DMF, or DMSO can be used. With respect to the reaction temperature, the reaction can be carried out at room temperature or under heating conditions, and may be carried out under cooling to regulate the reactivity.

It is preferable that the compound in the present embodiment be obtained as a crude by the reaction described above and be then further subjected to purification, thereby removing the residual metal impurities. That is, it is preferable to avoid residual metal impurities, from the viewpoint of preventing the deterioration of the resin with time, storage stability, and further, production yield due to processability, defects, and the like when the composition is formed into a resin and applied to a semiconductor production process. The metal impurities may be derived from the reaction aid in the production process of the compound, the reaction vessel for production, or other production equipment.

The residual amounts of the aforementioned metal impurities are preferably less than 1 ppm, more preferably less than 100 ppb, further preferably less than 50 ppb, still more preferably less than 10 ppb, and most preferably less than 1 ppb, based on the compound. In particular, with respect to metal species such as Fe, Ni, Sn, Zn, Cu, Sb, W, and Al which are classified as transition metals, there is concern that the residual amount of metals of 1 ppm or more may cause the denaturation and deterioration of materials with time due to the interaction with other compounds. Furthermore, with respect to alkaline metals or alkaline earth metals such as Na, K, Ca, and Mg, when the residual amount of metals contained in the resin is 1 ppm or more, there is a concern that the metal balance cannot be sufficiently reduced in the preparation of a resin for semiconductor process using the compound, which causes defects derived from residual metals in the semiconductor production process and reduction in the yield due to performance deterioration, and the characteristics are reduced due to the doping effect of a metal element on the substrate.

The purification method is not particularly limited, and the method described in International Publication No. WO 2015/080240, the method described in International Publication No. WO 2018/159707, or the like can be used. Specifically, the purification method comprises a step in which the compound is dissolved in an organic solvent that does not inadvertently mix with water to obtain an organic phase, the organic phase is brought into contact with an acidic aqueous solution to carry out extraction treatment, thereby transferring metals contained in the organic phase containing the compound and the organic solvent to an aqueous phase, and then, the organic phase and the aqueous phase are separated. The organic solvent that does not inadvertently mix with water is normally an organic solvent that is classified as a non-aqueous solvent. The organic solvent is not particularly limited, but is preferably an organic solvent that is safely applicable to semiconductor production processes. Normally, the amount of the organic solvent used is approximately 10% by mass relative to the compound used.

Specific examples of the organic solvent to be used include those described in International Publication No. WO 2015/080240. Among them, toluene, 2-heptanone, cyclohexanone, cyclopentanone, methyl isobutyl ketone, propylene glycol monomethyl ether acetate (PGMEA), ethyl acetate, and the like are preferable, and cyclohexanone and propylene glycol monomethyl ether acetate are particularly preferable.

The above acidic aqueous solution is arbitrarily selected from aqueous solutions in which generally known organic or inorganic compounds are dissolved in water. For example, examples thereof include those described in International Publication No. WO 2015/080240. These acidic aqueous solutions can be each used alone, or can also be used as a combination of two or more kinds. Examples of the acidic aqueous solution may include, for example, an aqueous mineral acid solution and an aqueous organic acid solution. Examples of the aqueous mineral acid solution may include, for example, an aqueous solution comprising one or more selected from the group consisting of hydrochloric acid, sulfuric acid, nitric acid, and phosphoric acid. Examples of the aqueous organic acid solution may include, for example, an aqueous solution comprising one or more selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid. Normally, the pH range of the acidic aqueous solution is about 0 to 5, and is more preferably about pH 0 to 3.

As other purification methods, a method using a filter, a method using an adsorptive ion exchange resin, a passing method using a column system, a method for conducting dispersion and suspension treatment of an ion exchange resin in a container, and a distillation method, which will be mentioned later, and the like can be arbitrarily used.

### [Filtering Purification Step (Passing Step)]

In the filter-passing step, the filter used for removing the metals in a solution containing the compound and the solvent can be one which is normally commercially available as a filter for liquid filtration. The filtering precision of the filter is not particularly limited, but the nominal pore size of the filter is preferably 0.2 μm or less, more preferably less than 0.2 μm, further preferably 0.1 μm or less, still more preferably less than 0.1 μm, and even more preferably 0.05 μm or less. The lower limit value of the nominal pore size of the filter is not particularly limited, but is normally 0.005 μm. The nominal pore size herein refers to the nominal pore size indicating the separation performance of a filter and is the pore size determined by test methods defined by the manufacturer of the filter, such as a bubble point test, a mercury intrusion porosimetry test, and a standard particle capture test. In the case of using a commercial product, it is the value described in the catalog data of the manufacturer. By setting the nominal pore size to 0.2 μm or less, the content of the metals after the solution is passed through the filter once can be effectively reduced. In the second embodiment, the filter-passing step may be carried out twice or more to reduce the content of each metal in the solution.

As the form of the filter, a hollow fiber membrane filter, a membrane filter, a pleated membrane filter, and a filter packed with a filtering medium such as a non-woven fabric, cellulose, and diatomaceous earth can be used. Among the above, it is preferable that the filter be one or more selected from the group consisting of a hollow fiber membrane filter, a membrane filter, and a pleated membrane filter. In particular, in terms of highly fine filtering precision and a high filtration area as compared to other forms, a hollow fiber membrane filter is particularly preferably used.

Examples of the filter material include polyolefin such as polyethylene and polypropylene; a polyethylene resin in which a functional group having ion exchange capacity is applied by graft polymerization; a polar group-containing resin such as polyamide, polyester, and polyacrylonitrile; and a fluorine-containing resin such as fluorinated polyethylene (PTFE). Among the above, the filtering medium of the filter is preferably one or more selected from the group consisting of a filtering medium made of polyamide, a filtering medium made of polyolefin resin, and a filtering medium made of fluorine resin. From the viewpoint of an effect of reducing heavy metals such as chrome, polyamide is particularly preferable. From the viewpoint of avoiding the dissolution of metals from the filtering medium, a filter having a filter material other than sintered metals is preferably used.

Examples of the polyamide filter include (hereinafter, trademarks), but are not limited to, Polyfix nylon series manufactured by KITZ MICROFILTER CORPORATION., Ultipleat P-Nylon 66 and Ultipor N66 manufactured by Nihon Pall Ltd., and LifeASSURE PSN series and LifeASSURE EF series manufactured by 3M Japan Limited.

Examples of the polyolefin filter include, but are not limited to, Ultipleat PE-Kleen and IonKleen manufactured by Nihon Pall Ltd., and Protego series, Microgard Plus HC10, and Optimizer D manufactured by Nihon Entegris G.K.

Examples of the polyester filter include, but are not limited to, Duraflow DFE manufactured by Central Filter Mfg Co Ltd., and a pleated type, PMC manufactured by Nihon Filter Co., Ltd.

Examples of the polyacrylonitrile filter include, but are not limited to, Ultrafilter AIP-0013D, ACP-0013D, and ACP-0053D manufactured by ADVANTEC TOYO KAISHA, LTD.

Examples of the fluorine resin filter include, but are not limited to, Emflon HTPFR manufactured by Nihon Pall Ltd., and LifeASSURE FA series manufactured by 3M Japan Limited.

These filters may be each used alone, or may also be used in combination of two or more kinds.

The filter may contain an ion exchanger such as a cation exchange resin, a cation charge regulator that generates a zeta potential in an organic solvent solution to be filtered, or the like.

Examples of the filter containing an ion exchanger include, but are not limited to, Protego series manufactured by Nihon Entegris G.K. and KURANGRAFT manufactured by Kurashiki Textile Manufacturing Co., Ltd.

Examples of the filter containing a material having a positive zeta potential such as a cationic polyamidepolyamine-epichlorohydrin resin include, but are not limited to: Zeta Plus 40QSH (registered trademark) and Zeta Plus 020GN (registered trademark) and LifeASSURE EF (registered trademark) series manufactured by 3M company.

[Treatment Step with Ion Exchange Resin]

Examples of other purification methods include a method for treating a solution containing the compound with an ion exchange resin. As the ion exchange resin, a known ion exchange resin having a function corresponding to the target metal element can be arbitrarily used. In the purification using the ion exchange resin, the product to be purified that contains the compound is subjected to ion adsorption by an ion exchange method or with a chelate group. Examples of the components removed in the treatment step with the ion exchange resin include, but are not limited to, acid components, and metal ions contained in metal components.

The method for performing the ion exchange method is not particularly limited, and a known method can be used. Typically, examples thereof include a method for allowing a solution containing the compound to pass through a packing part packed with the ion exchange resin. In addition, examples of the method for performing the ion exchange method include a method in which the ion exchange resin is added, dispersed, and suspended in a solution containing the compound in a treatment container, followed by separating and removing the ion exchange resin by a method such as filtration, thereby obtaining a solution subjected to purification treatment. In the treatment step with the ion exchange resin, the product to be purified may be subjected to treatment with the same ion exchange resin multiple times, or the product to be purified may be subjected to treatment with different ion exchange resins.

Examples of the ion exchange resin include a cation exchange resin and an anion exchange resin. It is preferable to use at least a cation exchange resin in terms of making it easy to adjust the content of metal components and set the mass ratio of the content of acid components to the content of metal components to the above range, and it is more preferable to use an anion exchange resin together with a cation exchange resin in terms of allowing the content of acid components to be adjusted. When both cation exchange resin and anion exchange resin are used, the solution may be pass through a packing part packed with a mixed resin containing both resins or may be pass through a plurality of packing parts packed with each resin.

As the cation exchange resin, a known cation exchange resin can be used, and above all, a gel cation exchange resin is preferable. Specific examples of the cation exchange resin include a sulfonic acid cation exchange resin and a carboxylic acid cation exchange resin. As the cation exchange resin, a commercial product can be used, and examples thereof include AMBERLITE IR-124, AMBERLITE IR-120B, AMBERLITE IR-200CT, ORLITE DS-1, and ORLITE DS-4 (hereinabove, manufactured by ORGANO CORPORATION), DUOLITE C20J, DUOLITE C20LF, DUOLITE C255LFH, and DUOLITE C-433LF (hereinabove, manufactured by Sumika Chemtex Company, Limited), DIAION SK-110, DIAION SK1B, and DIAION SK1BH (hereinabove, manufactured by Mitsubishi Chemical Group Corporation), and Purolite S957 and Purolite S985 (hereinabove, manufactured by Purolite).

As the anion exchange resin, a known anion exchange resin can be used, and above all, a gel anion exchange resin is preferably used. Herein, examples of the acid components present as ions in the product to be purified include inorganic acids derived from the catalyst upon production of the product to be purified and organic acids produced after the reaction upon production of the product to be purified (e.g., reaction raw materials, isomers, and by-products). Such acid components are classified into hard acids to acids having intermediate hardness in terms of HSAB (Hard and Soft Acids and Bases) theory. Thus, to increase the removal efficiency when removing these acid components by the interaction with an anion exchange resin, an anion exchange resin containing a hard base to a base having intermediate hardness is preferably used. The anion exchange resin containing a hard base to a base having intermediate hardness is preferably at least one anion exchange resin selected from the group consisting of a strongly basic anion exchange resin of type I having a trimethylammonium group, slightly strongly basic anion exchange resin of type II having a dimethylethanol ammonium group, and a weak basic anion exchange resin such as dimethylamine and diethylenetriamine. Among acid components, for example, organic acids are hard acids, and among inorganic acids, sulfuric acid ions are acids having intermediate hardness. Thus, use of a strongly basic or slightly strongly basic anion exchange resin mentioned above in combination with a weak basic anion exchange resin having intermediate hardness makes it easy to reduce the content of acid components to a preferred range.

As the anion exchange resin, a commercial product can be used, and examples thereof include AMBERLITE IRA-400J, AMBERLITE IRA-410J, AMBERLITE IRA-900J, AMBERLITE IRA67, ORLITE DS-2, ORLITE DS-5, and ORLITE DS-6 (manufactured by ORGANO CORPORATION), DUOLITE A113LF, DUOLITE A116, and DUOLITE A-375LF (manufactured by Sumika Chemtex Company, Limited), and DIAION SA12A, DIAION SA10A, DIAION SA10AOH, DIAION SA20A, and DIAION WA10 (manufactured by Mitsubishi Chemical Group Corporation). Among them, examples of the anion exchange resin containing a hard base to a base having intermediate hardness include ORLITE DS-6 and ORLITE DS-4 (hereinabove, manufactured by ORGANO CORPORATION), DIAION SA12A, DIAION SA10A, DIAION SA10AOH, DIAION SA20A, and DIAION WA10 (hereinabove, manufactured by Mitsubishi Chemical Group Corporation), and Purolite A400, Purolite A500, and Purolite A850 (hereinabove, manufactured by Purolite).

Ion adsorption with a chelate group can be carried out by using, for example, a chelate resin having a chelate group. Since chelate resin releases no alternative ions when capturing ions and no chemically highly active functional group such as a strongly acidic or strongly basic functional group is used, side reactions such as hydrolysis and condensation reactions on the organic solvent to be purified can be suppressed. Thus, purification can be carried out with higher efficiency. Examples of the chelate resin include resins having a chelate groups or chelating ability, such as an amide oxime group, a thiourea group, a thiouronium group, an iminodiacetic acid, amidephosphoric acid, phosphonic acid, aminophosphoric acid, aminocarboxylic acid, N-methylglucamine, an alkylamino group, a pyridine ring, cyclic cyanine, a phthalocyanine ring, and a cyclic ether.

As the chelate resin, a commercial product can be used, and examples thereof include DUOLITE ES371N, DUOLITE C467, DUOLITE C747UPS, SUMICHELATE MC760, SUMICHELATE MC230, SUMICHELATE MC300, SUMICHELATE MC850, SUMICHELATE MC640, and SUMICHELATE MC900 (hereinabove, manufactured by Sumika Chemtex Company, Limited), and Purolite S106, Purolite S910, Purolite S914, Purolite S920, Purolite S930, Purolite S950, Purolite S957, and Purolite S985 (hereinabove, manufactured by Purolite).

The method for performing ion adsorption is not particularly limited, and a known method can be used. Typically, examples thereof include a method for allowing the product to be purified to pass through a packing part packed with the chelate resin. In the treatment step with the ion exchange resin, the product to be purified may be passed through the same chelate resin multiple times, or the product to be purified may be passed through different chelate resins.

The packing part normally contains a container, and the aforementioned ion exchange resin packed in the container. Examples of the container include a column, a cartridge, and a packed column, but the container may be a container other than those exemplified above, as long as the product to be purified can pass therethrough after the above ion exchange resin is packed.

### [Distillation Step]

Examples of other purification methods include a method for distilling the compound itself. The distillation method is not particularly limited, but a known method such as normal pressure distillation, vacuum distillation, molecular distillation, or steam distillation can be used.

### [Preferred Production Method]

(Compound in which RG is a Benzene Ring)

The method for producing the compound of the formula (Bz) will be specifically described. The compound of the formula (Bz) preferably uses the compound represented by the formula (MB) as a raw material. The substituent, r1, r2, and the like in the compound are defined as mentioned above. R¹, R, and OH are each bonded to any bondable position. Provided that r1 and r2 in the formula (MB) are selected so that the sum of r1 to r4 satisfies the valence of the benzene ring, in the formula (Bz). Examples of the compound of the formula (MB) include hydroxybenzaldehyde.

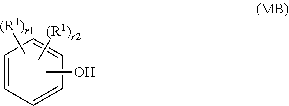

The compound of the formula (Bz) is produced by various methods, but from the viewpoint of the availability of raw materials and the yield, it is preferably produced by the method comprising the following steps:
a provision step of providing a compound represented by the formula (MB),
an iodination step of introducing iodine into the compound,
a protective group introduction step of introducing a protective group into the compound, and
a reduction step of reducing the compound.

From the viewpoint of suppressing by-products, it is preferable to conduct the provision step, the iodination step, the protective group introduction step, and the reduction step in the order presented.

### 1) Iodination Step

Examples of the solvent that may be used in the iodination step include a wide variety of solvents including a polar aprotic solvent and a protic polar solvent. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar protic solvent or a mixture thereof is preferable, and from the viewpoint of suppressing side reactions, a mixture of a polar protic solvent and water is preferable. The solvent is effective, but is not essential. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol. The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

The reaction mixture is formed by adding the raw material compound, the catalyst, and the solvent to a reactor. Any suitable reactor is used. The reaction may be carried out by arbitrarily selecting a known method such as a batch method, a semi-batch method, or a continuous method. The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable, a temperature of 0° C. to 70° C. is more preferable, and a temperature of 0° C. to 50° C. is further preferable. The preferred temperature range in the reaction in the present aspect is 0° C. to 100° C. The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation. The preferred reaction pressure in the reaction in the present aspect is reduced pressure to normal pressure, and reduced pressure is preferable. The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes. The preferred reaction time range in the reaction in the present aspect is 15 minutes to 600 minutes. Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity compound by a separation and purification method by filtration, concentration, distillation, extraction, crystallization, recrystallization, column chromatography, activated carbon, or the like, which are well-known purification methods in the art, or a combined method thereof.

### 2) Protective Group Introduction Step

As the solvent that may be used in the present step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol. The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials. As the reagent for introducing protection, a wide variety of reagents for introducing protection which function under the reaction conditions of the present embodiment are used. Examples of the suitable reagent for introducing protection include, but are not limited to, acid halides, acid anhydrides, active carboxylic acid derivative compounds such as dicarbonate, alkyl halides, vinyl alkyl ethers, dihydropyran, and halocarboxylic acid alkyl esters. As the catalyst that may be used in the protection step, a wide variety of protection catalysts which function under the reaction conditions of the present embodiment are used. An acid catalyst or a base catalyst is preferable. Examples of suitable acid catalysts include, but are not limited to, an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. These acid catalysts are used alone as one kind or in combination of two or more kinds. Among them, organic acids and solid acids are preferable from the viewpoint of production, and it is preferable to use hydrochloric acid or sulfuric acid from the viewpoint of production such as easy availability and handleability. Examples of suitable base catalysts include, but are not limited to, an amine-containing catalyst such as pyridine and ethylenediamine, and non-amine basic catalyst such as a metal salt, and in particular, potassium salt or acetate is preferable. Examples of suitable catalysts include, but are not limited to, potassium acetate, potassium carbonate, potassium hydroxide, sodium acetate, sodium carbonate, sodium hydroxide, and magnesium oxide. All non-amine base catalysts of the present embodiment are commercially available from, for example, EM Science (Gibbstown) or Aldrich (Milwaukee). The amount of the catalyst used can be arbitrarily set according to, for example, the substrate, catalyst, and reaction conditions to be used, without particular limitation. In general, it is suitably 1 to 5000 parts by mass, and from the viewpoint of the yield, preferably 50 to 3000 parts by mass, based on 100 parts by mass of the reaction raw materials.

The reaction mixture is formed by adding the compound for protection, the catalyst, and the solvent to a reactor. Any suitable reactor is used. The reaction may be carried out by arbitrarily selecting a known method such as a batch method, a semi-batch method, or a continuous method. The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 10° C. to 190° C. is preferable, a temperature of 25° C. to 150° C. is more preferable, and a temperature of 50° C. to 100° C. is further preferable. The preferred temperature range in the reaction in the present aspect is 0° C. to 100° C. The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation. The preferred reaction pressure in the reaction in the present aspect is reduced pressure to normal pressure, and reduced pressure is preferable. The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes. The preferred reaction time range in the reaction in the present aspect is 15 minutes to 600 minutes. Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### 3) Reduction Step

As the solvent that may be used in the reducing step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable, and from the viewpoint of suppressing side reactions, a mixture of a polar aprotic solvent and a polar protic solvent is preferable. As the polar protic solvent, water, or an alcohol solvent such as methanol, ethanol, propanol, and butanol is further preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol. The amount of the solvent used can be arbitrarily set according to, for example, the substrate, reducing agent, and reaction conditions to be used, without particular limitation. In general, it is suitably 0 to 10000 parts by mass, and from the viewpoint of the yield, it is preferably 100 to 2000 parts by mass, based on 100 parts by mass of the reaction raw materials.

As the reducing agent, a wide variety of reducing agents which function under the reaction conditions of the present embodiment are used. Examples of suitable reducing agents include, but are not limited to, a metal hydride and a metal hydride complex compound, such as borane dimethylsulfide, diisobutylaluminum hydride, sodium borohydride, lithium borohydride, potassium borohydride, zinc borohydride, lithium tri-s-butylborohydride, potassium tri-s-butylborohydride, lithium triethylborohydride, lithium aluminum hydride, lithium tri-t-butoxyaluminum hydride, and sodium bis(methoxyethoxy)aluminum hydride.

The amount of the reducing agent used can be arbitrarily set according to, for example, the substrate, reducing agent, and reaction conditions to be used, without particular limitation. In general, it is suitably 1 to 500 parts by mass, and from the viewpoint of the yield, it is preferably 10 to 200 parts by mass, based on 100 parts by mass of the reaction raw materials.

As a quenching agent, a wide variety of quenching agents which function under the reaction conditions of the present embodiment are used. The quenching agent has a function of deactivating the reducing agent. The quenching agent is effective, but is not an essential component. Examples of suitable quenching agents include, but are not limited to, ethanol, aqueous ammonium chloride solution, water, hydrochloric acid, and sulfuric acid. The amount of the quenching agent used can be arbitrarily set according to the amount of the reducing agent to be used, without particular limitation. In general, it is suitably 1 to 500 parts by mass, and from the viewpoint of the yield, it is preferably 50 to 200 parts by mass, based on 100 parts by mass of the reaction raw materials.

The reaction mixture is formed by adding the compound to be reduced, the reducing agent, and the solvent to a reactor. Any suitable reactor is used. The reaction may be carried out by arbitrarily selecting a known method such as a batch method, a semi-batch method, or a continuous method. The reaction temperature is not particularly limited. The preferred range varies according to the concentration of the substrate, the stability of the formed product, the selection of the reducing agent, and the desired yield. In general, a temperature of 0° C. to 200° C. is suitable, and from the viewpoint of the yield, a temperature of 0° C. to 100° C. is preferable, a temperature of 0° C. to 70° C. is more preferable, and a temperature of 0° C. to 50° C. is further preferable. The preferred temperature range is 0° C. to 100° C. The reaction pressure is not particularly limited. The preferred range varies according to the concentration of the substrate, the stability of the formed product, the selection of the reducing agent, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation. The preferred reaction pressure in the reaction in the present aspect is reduced pressure to normal pressure, and reduced pressure is preferable. The reaction time is not particularly limited. The preferred range varies according to the concentration of the substrate, the stability of the formed product, the selection of the reducing agent, and the desired yield. However, the majority of the reactions are carried out for less than 6 hours, and the reaction time is typically 15 minutes to 600 minutes. The preferred reaction time range in the reaction in the present aspect is 15 minutes to 600 minutes. Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity compound by a separation and purification method by filtration, concentration, distillation, extraction, crystallization, recrystallization, column chromatography, activated carbon, or the like, which are well-known purification methods in the art, or a combined method thereof.

(Compound in which RG is a Naphthalene Ring)

The method for producing the compound of the formula (N) will be specifically described. The compound of the formula (N) preferably uses the compound represented by the formula (MN) as a raw material. In the formula, the substituent, s3, s4, and the like are defined as mentioned above. Provided that s3 and s4 in the formula (MN) are selected so that the sum of s1 to s4 satisfies the valence of naphthalene in the formula (N). Examples of R¹ include, but are not limited to, a hydroxy group, an amino group, a nitro group, a halogen atom other than iodine, and an aldehyde group. Specific examples of the compound of the formula (MN) include, but are not limited to, (di)hydroxynaphthaldehyde, aminonaphthaldehyde, nitronaphthaldehyde, and naphthaldehyde chloride.

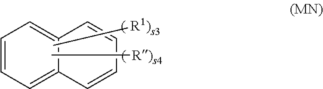

The compound of the formula (N) is produced by various methods, but from the viewpoint of the availability of raw materials and the yield, it is preferably produced by the method comprising the following steps.

From the viewpoint of the availability of raw materials and the yield, the compound of the formula (N) is preferably produced by the method comprising the following steps:
a provision step of providing a compound represented by the formula (MN),
an iodination step of introducing iodine into the compound,
a protective group introduction step of introducing a protective group into the compound, and
a reduction step of reducing the compound.

From the viewpoint of suppressing by-products, it is preferable to conduct the provision step, the iodination step, the protective group introduction step, and the reduction step in the order presented, or the provision step, the protective group introduction step, the iodination step, and the reduction step in the order presented. The solvent and reaction conditions that can be used in each step are as described in the method for producing a compound in which RG is a benzene ring.

(Compound in which RG is an Adamantane Ring)

The method for producing the compound of the formula (Ad) will be specifically described. In the present method, the compound represented by the formula (MA) is preferably used as a raw material. In the formula (MA), R¹, R″, t2, and t3 are as defined in the formula (Ad). Provided that t2 and t3 in the formula (MA) are selected so that the sum of t1 to t3 satisfies the valence of adamantane in the formula (Ad). Examples of the compound include, but are not limited to, adamantane triol.

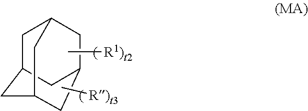

The compound of the formula (Ad) is produced by various methods, but from the viewpoint of the availability of raw materials and the yield, it is preferably produced by the method comprising the following steps:
a provision step of providing a compound represented by the formula (MA), and
an iodination step of introducing iodine.

As the solvent that can be used in the iodination step, those exemplified in the method for producing a compound in which RG is a benzene ring can be used. In the present step, the reaction mixture is formed by adding the raw material compound, the catalyst, and the solvent to a reactor. The reaction conditions and the like are as described in the method for producing a compound in which RG is a benzene ring.

The iodination step preferably comprises distilling off water to concentrate a reaction solution, in the reaction for obtaining alkyl iodide using an aqueous hydrogen iodide solution and adamantane alcohol as raw materials. The hydrogen iodide concentration in the reaction solution is preferably 10% or more, more preferably 25% or more, further preferably 40% or more, particularly preferably 45% or more, and most preferably 50% or more. When the reaction solution is separated to two or more phases, the hydrogen iodide concentration of the aqueous phase that contains hydrogen iodide preferably has the above concentration.

Adamantane alcohol may have a hydroxy group or two or more hydroxy groups in a molecule. The hydroxy group to be iodinated may be any of primary, secondary, and tertiary hydroxy groups, and is preferably a secondary or tertiary hydroxy group, and is more preferably a tertiary hydroxy group.

Adamantane alcohol is preferably represented by the following formula (MA-1). In the formula, R¹ and R″ are as defined in the formula (Ad). Provided that, R¹ is preferably a monovalent group having 1 to 12 carbon atoms and optionally containing —OH, —NO₂, or at least one functional group. The functional group is one or more groups selected from the group consisting of a hydroxy group, an ether group, an ester group, a carboxyl group, a halogen atom, —NO₂, and NLL′. In addition, L and L′ are each independently a monovalent group having 1 to 12 carbon atoms and optionally containing a hydrogen atom, a hydroxy group, or at least one functional group.

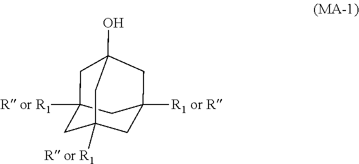

The hydrogen iodide is preferably 1.01 equivalents or more, more preferably 1.1 equivalents or more, further preferably 1.3 equivalents or more, and particularly preferably 1.5 equivalents or more based on the hydroxy group to be iodinated, in a mole ratio.

When the compound of the formula (MA-1) has two or more hydroxy groups in a molecule, all the hydroxy groups may be iodinated, or one or more hydroxy groups may be remained. Examples of the method for allowing one or more hydroxy groups to remain include use of a hydrophobic solvent. The hydrophobic solvent refers to a solvent that does not mix with water in any proportion. In the reaction system in which an aqueous hydrogen halide solution and a hydrophobic solvent are separated into two liquid-liquid phases of an aqueous phase and a hydrophobic solvent phase, iodination of hydroxy groups progresses in the aqueous phase. Among alcohols having two or more hydroxy groups, alkyl iodide in which one or more hydroxy groups are remaining is extracted in the hydrophobic solvent phase, so that alkyl iodide in which one or more hydroxy groups are remaining can be obtained. In addition, extraction of the produced alkyl iodide in the hydrophobic solvent can suppress the reduction in the yield due to side reactions, and thus, use of a hydrophobic solvent is effective also in the case of iodinating all the hydroxy groups.

The hydrophobic solvent may be or may not be azeotropic with water, and a hydrophobic solvent azeotropic with water is preferable. Examples of the hydrophobic solvent azeotropic with water include dichloromethane, chloroform, carbon tetrachloride, nitromethane, 1,2-dichloroethane, ethyl acetate, butyl acetate, methyl ethyl ketone, methyl isobutyl ketone, pentane, cyclohexane, hexane, benzene, toluene, o-xylene, m-xylene, p-xylene, cumene, nitrobenzene, phenol, s-butanol, cyclopentyl methyl ether, and cyclohexanone, and use of hexane, toluene, o-xylene, m-xylene, or p-xylene is preferable. The hydrophobic solvent may be used alone, or may also be used in combination of two or more hydrophobic solvents.

The hydrophobic solvent is preferably 50 equivalents or less, more preferably 30 equivalents or less, and further preferably 20 equivalents or less, based on the alcohol of the raw material, in a mass ratio.

In the reaction, acid may be used in combination. Examples of the type of acid include sulfuric acid, nitric acid, phosphoric acid, p-toluenesulfonic acid, methanesulfonic acid, benzenesulfonic acid, acetic acid, trifluoroacetic acid, citric acid, oxalic acid, malic acid, lactic acid, glycolic acid, succinic acid, chrome acid, and boric acid.

In the reaction, metal iodide may be used in combination. For example, use of LiI, NaI, KI, MgI₂, CaI₂, or AlI₃ in combination is effective.

To prevent bumping, the reaction solution is preferably stirred in the reaction. Stirring blades having various shapes can be suitably used, and examples thereof include a flat paddle blade, an inclined paddle blade, a turbine blade, a disk turbine blade, a propeller blade, a three retreat blade, an anchor blade, a helical ribbon blade, a screw blade, an anchor blade, MAXBLEND, FULLZONE, and TWINSTIR.

The stirring speed may be any speed. When a hydrophobic solvent is used and the reaction solution is separated into two liquid-liquid phases, the stirring speed may be a stirring speed at which the interface trembles, the stirring speed at which some oil droplets or water droplets are produced and dispersed, or the stirring speed that leads to a totally dispersed state.

The reaction temperature is preferably 0 to 150° C., more preferably 20 to 150° C., and further preferably 50 to 120° C. On the other hand, to obtain alkyl iodide in high yield, it is required to distill off water in the reaction to concentrate the reaction solution. To distill off water, the reaction temperature is required to be set to the boiling point of the reaction solution. When the boiling point varies with the use of the hydrophobic solvent, the reaction temperature can be controlled by carrying out the reaction at reduced pressure or increased pressure.

The reaction temperature can be controlled also by changing the stirring speed. Typically, when the hydrophobic solvent is azeotropic with water, the azeotropic point thereof is lower than the boiling point of the solvent. When a hydrophobic solvent azeotropic with water is used and the reaction solution is separated into two liquid-liquid phases, the higher the stirring speed is, the more two phases approach a totally dispersed state, and accordingly, the boiling point approaches the azeotropic point. Thus, the reaction temperature can be controlled by the stirring speed.

When the reaction solution is concentrated, the total amount of water distilled by simple distillation may be distilled off, or a required amount thereof may be distilled off by using a Dean-Stark apparatus or the like, and a required amount thereof is preferably distilled off by using a Dean-Stark apparatus or the like.

The amount of water to be distilled off is preferably determined so that the hydrogen iodide concentration can be maintained at a predetermined concentration or more. The above concentration is preferably a concentration 15% lower than the hydrogen iodide concentration charged or more, more preferably a concentration 10% lower than the hydrogen halide concentration charged or more, further preferably a concentration 5% lower than the hydrogen iodide concentration charged or more, and particularly preferably the hydrogen iodide concentration charged or more. In addition, a certain amount of water may be continuously distilled off, or water may be collectively distilled off every predetermined time. After completion of the reaction, an operation of purifying and isolating alkyl iodide is conducted.

Oxidation of hydrogen iodide in the reaction produces iodine as a simple substance. Since the remaining of iodine as a simple substance is a cause of coloration and the like, iodine as a simple substance is preferably reduced to hydrogen iodide by the reducing agent. Examples of the type of the reducing agent include, but are not particularly limited to, sodium sulfite, sodium hydrogen sulfite, and phosphinic acid.

The reducing agent may be directly put in the reaction solution, or may be put as an aqueous solution. The reducing agent may be put in a state where hydrogen iodide remains in the reaction solution, or may be put after hydrogen iodide is neutralized with a base. Examples of the base used in the neutralization operation include, but are not particularly limited to, sodium hydroxide, potassium hydroxide, calcium hydroxide, sodium carbonate, and sodium bicarbonate.

In the case of using a hydrophobic solvent, purification can be carried out by washing the hydrophobic solvent phase with water. In washing with water, for example, pure water, an aqueous sodium chloride solution, an aqueous solution of nitric acid, an aqueous solution of oxalic acid, an aqueous solution of sulfuric acid, or an aqueous hydrogen chloride solution can be suitably used. After completion of the reaction, washing with water may be performed by adding a hydrophobic solvent. The hydrophobic solvent added after completion of the reaction may be the same as or different from the hydrophobic solvent used in the reaction.

In general, washing with water is conducted in the vicinity of room temperature, but when the product is precipitated upon washing with water at room temperature, washing with water can be conducted while heating. The temperature of washing with water is preferably the azeotropic temperature of the hydrophobic solvent and water or less.

As mentioned above, purification can be performed by passing through an ion exchange resin, a chelate resin, a metal removal filter, a fine particle removal filter, or the like. The ion exchange resin, the chelate resin, the metal removal filter, and the fine particle removal filter may be applied singly or in combination with an operation such as washing with water, upon purification.

Isolation of the compound of the formula (Ad) can be carried out by distillation or crystallization. When isolation is carried out by distillation, the distillation method is not particularly limited, and for example, methods such as batch simple distillation, equilibrium flash distillation, batch rectification, and continuous rectification can be suitably applied. The compound of the formula (Ad) may also be distilled and recovered, or may be recovered as a waste solution or a bottom solution.

When isolation is carried out by crystallization, the hydrophobic solvent used as the solvent in the reaction may be used as it is, or a new solvent may be added. The solvent may be a single solvent, or two or more solvents may be used in combination.

The solvent upon crystallization is preferably 20 equivalents or less, more preferably 10 equivalents or less, further preferably 5 equivalents or less, and particularly preferably 3 equivalents or less based on the compound of the formula (Ad), in a mass ratio. The ratio of the solvent to the compound of the formula (Ad) can be regulated by distilling of the solvent by distillation.

Crystal may be precipitated by adding a seed crystal, or crystal may be precipitated by cooling the solution without adding a seed crystal. After precipitation of crystal, the slurry is cooled to improve the yield. The cooling rate is preferably 30° C./h or less, more preferably 20° C./h or less, further preferably 10° C./h or less, and particularly preferably 5° C./h or less.

The temperature at which the slurry is subjected to solid liquid separation after cooling is preferably −50 to 40° C., more preferably −20 to 30° C., and further preferably −20 to 10° C. The retention time from the slurry temperature reached the temperature at which the slurry is subjected to solid liquid separation until the slurry is subjected to solid liquid separation is not particularly limited, and is preferably within 24 hours, and more preferably within 10 hours.

The method for solid liquid separation is not particularly limited, and for example, methods such as nutsche filtration, centrifugation, and pressure filtration can be suitably applied.

When the compound (MA) is iodinated, a base or an oxidizing agent can be used. When the activity of the base or the oxidizing agent is high, a compound (Da2) can be synthesized. Examples of the base include sodium hydroxide, potassium hydroxide, lithium hydroxide, sodium carbonate, potassium carbonate, calcium carbonate, sodium bicarbonate, and potassium bicarbonate. Examples of the oxidizing agent include, but are not particularly limited to, periodic acid, hydrogen peroxide, and a predetermined additive (such as hydrochloric acid, sulfuric acid, nitric acid, and p-toluenesulfonic acid). Further, the compound (Da2) can be synthesized by condensing hydroxy groups of the compound (MA) each other using strong acid and the like.

### 4. Composition

The compound is effective as a composition. Since the compound is useful particularly as a composition for lithography, the composition containing the compound will be described below, by way of the composition for lithography.

The compound expresses the sensitizing effect of the composition for lithography containing this compound in radiation irradiation. This reason is not limited, but is considered that the compound promotes the absorption of radiation. This effect is particularly significant in extreme ultraviolet ray (EUV) irradiation. The content of the sensitizing effect includes a plurality of forms. For example, it can be confirmed as follows, when a photosensitive layer formed by using the composition for lithography is used as a resist film for lithography. 1) The film is exposed by a surface exposure method using no pattern, then subjected to a PEB step (a step of carrying out heat treatment after exposure) if required, and a development step (a step of dissolving and removing the exposed portion or the unexposed portion with a developing solution) if required, and the film thickness of the film thus obtained is measured. 2) The film thickness of films obtained by changing the exposure amount are measured, and the exposure amount at which the film thickness varies rapidly is defined as the sensitivity in the surface exposure method. 3) When the sensitivity is confirmed on a low exposure amount side, the sensitizing effect is recognized. In the method for forming a pattern by exposure, 1) patterns are formed by changing the exposure amount, and the exposure amount at which a prescribed line width is achieved after exposure is defined as the sensitivity. 2) When the sensitivity is confirmed on a lower exposure amount side, the sensitizing effect is recognized. In addition, particularly in the pattern evaluation with an extreme ultraviolet ray (EUV), the sensitivity can also be confirmed by the reduction in defects such as pitting and bridges. These defects result from the optical fluctuation of the exposure amount or an exposure state with a low exposure amount substantially similar to deficiency. When a resist film has the sensitizing effect, the fluctuation and deficiency are avoided by absorption promotion, and the defects are reduced. When the compound is used as a composition for lithography, the compound can be directly used as a constitutional component of the composition. As another method, the compound can also be processed into a resin containing the compound as a partial structure (a base material (A)), an additive (an acid generating agent (C), a crosslinking agent (G), an acid diffusion controlling agent (E), or a further component (F), or the like, and used as a composition for lithography having the resin or the additive as a constitutional component.

The composition for lithography according to the present embodiment contains a compound represented by the formula (1) (hereinafter, also referred to as “the compound (B)”), and if required, may contain other components such as the base material (A), a solvent (S), the acid generating agent (C), the crosslinking agent (G), or the acid diffusion controlling agent (E). Hereinafter, each of these components will be described.

### [Compound (B)]

The composition in the present embodiment contains one or more compounds (B). The composition preferably contains two or more kinds of compounds (B), without limitation. When two or more kinds of compounds (B) are contained, the etching defect shown in Examples, which will be mentioned later, tends to be reduced. The reason why the etching defect is reduced is not clear, but for example, it is considered that the compatibility of the compound (B) in the composition is improved and fine defects may be reduced when a film is formed.

The amount of the compound (B) blended is not limited. When the amount of the compound (B) blended is low (this compound is referred to as the compound (B′)), the amount of the compound (B′) is preferably 1 ppm or more, and more preferably 10 ppm or more in the total amount of the compound (B), from the viewpoint of the effect of improving the etching defect. When there is a compound (B) which is blended the most (this compound is referred to as the compound (B″)), the content of the compound (B″′) having a lower content of iodine atoms in the molecule than the compound (B″) is preferably 40% by mass or less, further preferably 10% by mass or less, and most preferably 5% by mass or less in the total compound (B), from the viewpoint of improving the sensitivity.

In one aspect, when a monomer compound having a large number of iodine is set to H, a monomer compound having a small number of iodine is set to L, and a dimer compound is set to D, the following combinations can be exemplified in the compound (B).

H/L(mass ratio, the same applies to hereinafter)=(99 to 99.9):(1 to 0.1)

H/D=(99 to 99.9):(1 to 0.1)

H/L/D=(98 to 99.9):(1 to 0.05):(1 to 0.05)

The method for mixing two or more kinds of compounds (B) is not limited, and two or more kinds of compounds (B) may be mixed, or may be synthesized as a mixture at the same time in the process of synthesizing the compound (B).

Examples of the more preferred aspect of the compound B include the followings.
1) A combination of the compound of the formula (1) as the standard and a compound that is represented by the formula (1), but has a smaller number of iodine atoms than the compound as the standard (preferably a compound of the formula (BP0-1), which will be mentioned later).
2) A combination of the compound of the formula (1) as the standard and a polymer of the compound represented by the formula (1) (preferably the aforementioned formula (DM0-1)).
3) A combination of the compound of the formula (1) as the standard, the compound having a small number of iodine atoms, and the polymer.

It is presumed that the composition has a high effect particularly with respect to ensuring the stability with time resulting from inorganic materials or inorganic components by containing the compound of the formula (DM0-1). It is presumed that a high trap effect on the causative components leads to the improvement of stability with time. In addition, as another preferred aspect, it is presumed that, since the composition contains the compound represented by the formula (BP0-1), the mechanism resulting from the difference in the oxidation reduction potential with the compound represented by the formula (1) is effective to ensure stability with time and leads to the improvement of the stability with time resulting from natural oxidation and the deterioration with time of coexisting materials.

The compound of the formula (DM0-1) is as mentioned above. Examples of the compound having a small number of iodine atoms include the compound represented by the formula (BP0-1).

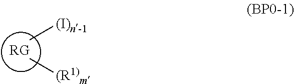

RG, I, and R¹ are as defined in the formula (1). n′ is an integer of 1 to 5 and n or less. m′ is an integer of 1 to 5 and m or less. When n′ is 2 to 5, the compound of the formula (BP0-1) is one of the compounds represented by the formula (1).

The compound of the formula (BP0-1) is preferably represented by the following formulas. In the formula, R, R¹, R″, A, r1 to r4, s2 to s3, and t2 to t3 are defined as mentioned above. a1 and r4a are an integer of 0 to 4, a1 and r4a are a number satisfying a1+r4a r4. Although r4 is defined as mentioned above, r4 preferably has the same meaning as r4 in the formula (Bz). s1b is an integer of 0 to 6, and is an integer satisfying s1b≤(s1−1). Although s1 is defined as mentioned above, s1 preferably has the same meaning as si in the formula (N). t1b is an integer of 0 to 9, and is an integer satisfying t1b≤(t1−1). Although t1 is defined as mentioned above, t1 preferably has the same meaning as t1 in the formula (Ad).

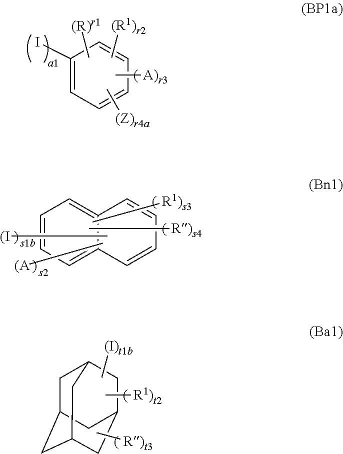

The composition using the compound of the formula (1) and the formula (DM0-1) or the formula (BP0-1) in combination is excellent in storage stability. This cause is not limited, but it can be inferred that the compound of the formula (DM0-1) or the formula (BP0-1) sterically or electronically captures causative materials and causative components that deteriorate storage stability. From such a viewpoint, the lower limit value of the total amount of the compounds represented by the formula (DM0-1) and the formula (BP0-1) is preferably 1 ppm or more, more preferably 2 ppm or more, further preferably 5 ppm or more, and particularly preferably 10 ppm or more, based on the total compounds represented by the formula (1). The upper limit value of the total amount is preferably 10000 ppm or less, more preferably 8000 ppm or less, further preferably 5000 ppm or less, and particularly preferably 3000 ppm or less.

From the viewpoint of increasing the thermal resistance of the compound in addition to the above effect, the compound of the formula (DM0-1) is preferably used, and the compound of the formula (DM1a), (Dn1), or (Da1) is more preferable. Among them, a dimer is particularly preferable.

From the viewpoint of increasing the dissolution stability of the compound in addition to the above effect, the compound of the formula (BP0-1) is preferably used, and a compound having a small number of iodine atoms is preferably used as the compound of the formula (BP1a), (BP2a), (Bn1), or (Ba1). Even when the compound of the formula (BP1a), (BP2a), (Bn1), or (Ba1) contains no iodine atoms, the expected effect is exhibited. In the present aspect, z in the formula (BP1a) is capable of containing no I. Hereinafter, preferred compounds will be described.

The compound represented by the formula (BP1a) is preferably a compound represented by the formula (BP1b).

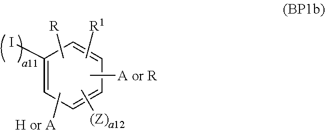

In the formula (BP1b), I, R, R¹, A, and Z are as defined in the formula (BP1a), and a11 and a12 are an integer of 0 to 5 satisfying a11+a12≤r4. Although r4 is defined as mentioned above, r4 preferably has the same meaning as r4 in the formula (Bz) (the same applies hereinafter).

The compound represented by the formula (BP1b) is preferably a compound represented by the formula (BP1c1).

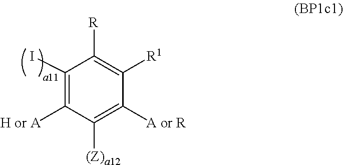

In the formula (BP1c1), I, R, R¹, A, and Z are as defined in the formula (BP1a), and a11 and a12 are an integer of 0 to 5 satisfying a11+a12≤r4.

The compound represented by the formula (BP1c1) is preferably a compound represented by the formula (BP1d11).

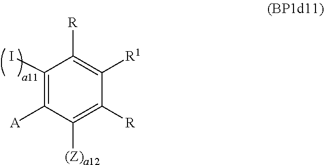

In the formula (BP1d11), I, R, R¹, A, and Z are as defined in the formula (BP1a), and a11 and a12 are an integer of 0 to 5 satisfying a11+a12≤r4.

The compound represented by the formula (BP1c1) is preferably a compound represented by the formula (BP1d12).

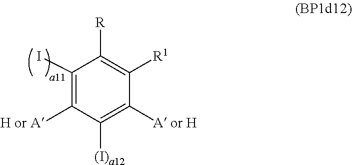

In the formula (BP1d12), I, R, R¹, and Z are as defined in the formula (BP1a), and a11 and a12 are an integer of 0 to 5 satisfying a11+a12≤r4. A′ is a group having a protective group, and represented by —O—R^{a}—O—R^{b}, —O—CO—O—R^{b}, or —O—R^{a}—CO—O—R^{b}, or —O—R^{a}—O—CO—R^{b}. R^{a} is a linear or branched alkyl group having 1 to 3 carbon atoms. R^{b} is a monovalent linear, branched, or cyclic alkyl group having 1 to 3 carbon atoms or a divalent cyclic alkyl group, and forms a ring with an adjacent oxygen atom. A cyclic structure containing R^{a} and R^{b} may be formed. Provided that, one or more A′ are present.

The compound represented by the formula (BP1b) is preferably a compound represented by the formula (BP1c2).

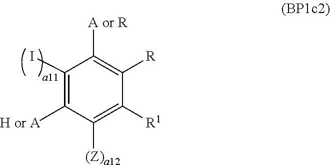

In the formula (BP1c2), I, R, R¹, A, and Z are as defined in the formula (BP1a), and a11 and a12 are an integer of 0 to 5 satisfying a11+a12≤r4.

The compound represented by the formula (BP1c2) is preferably a compound represented by the formula (BP1d21).

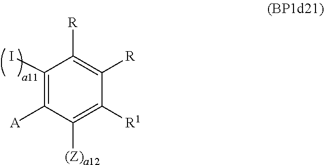

In the formula (BP1d21), I, R, R¹, A, and Z are as defined in the formula (BP1a), and a11 and a12 are an integer of 0 to 5 satisfying a11+a12≤r4.

The compound represented by the formula (BP1c1) is preferably a compound represented by the formula (BP1d22).

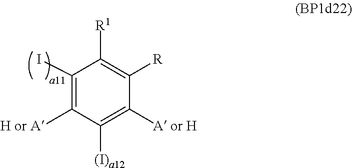

In the formula (BP1d22), I, R, R¹, and Z are as defined in the formula (BP1a), and a11 and a12 are an integer of 0 to 5 satisfying a11+a12≤r4. A′ is as defined in the formula (BP1d12).

The compound represented by the formula (BP1b) is preferably a compound represented by the following formula (BP1c3).

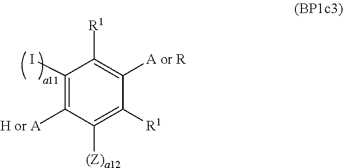

In the formula (BP1c3), I, R, R¹, A, and Z are as defined in the formula (BP1a), and a11 and a12 are an integer of 0 to 5 satisfying a11+a12≤r4.

The compound represented by the formula (BP1c3) is preferably a compound represented by the formula (BP1d31).

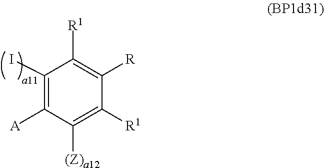

In the formula (BP1d31), I, R, R¹, A, and Z are as defined in the formula (BP1a), and a11 and a12 are an integer of 0 to 5 satisfying a11+a12≤r4.

The compound represented by the formula (BP1b) is preferably a compound represented by the formula (BP1c4).

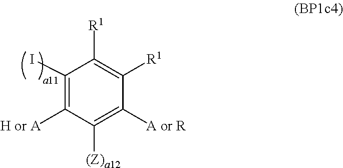

In the formula (BP1c4), I, R, R¹, A, and Z are as defined in the formula (BP1a), and a11 and a12 are an integer of 0 to 5 satisfying a11+a12≤r4.

The compound represented by the formula (BP1c4) is preferably a compound represented by the following formula (BP1d41).

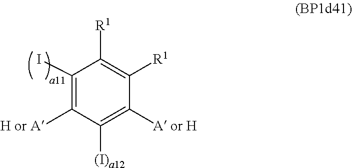

In the formula (BP1d41), I, R, R¹, and A are as defined in the formula (BP1a), and a11 and a12 are an integer of 0 to 5 satisfying a11+a12≤r4. A′ is as defined in the formula (BP1d12).

The compound represented by the formula (Bn1) is preferably a compound represented by the formula (Bn1a).

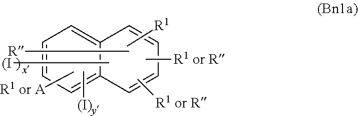

In the formula (Bn1a), I, R¹, R″, and A are as defined in the formula (Bn1) Each of x′ and y′ is 0 or 1, and x′ and y′ satisfy (x′+y′)≤(x+y−1), with respect to x and y in the formula (1n).

The compound represented by the formula (Bn1a) is preferably a compound represented by the formula (Bn1b1).

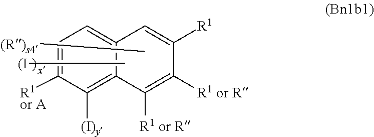

In the formula (Bn1b1), I, R¹, R″, and A are as defined in the formula (Bn1) Each of x′ and y′ is 0 or 1, and x′ and y′ satisfy (x′+y′)≤(x+y−1), with respect to x and y in the formula (1n). s4′ is defined as mentioned above.

The compound represented by the formula (Bn1b1) is preferably a compound represented by the following formula (Bn1c11).

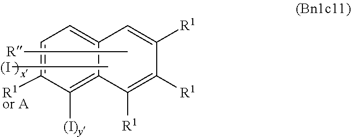

In the formula (Bn1c11), I, R¹, R″, and A are as defined in the formula (Bn1). Each of x′ and y′ is 0 or 1, and x′ and y′ satisfy (x′+y′)≤(x+y−1), with respect to x and y in the formula (1n).

The compound represented by the formula (Bn1b1) is preferably a compound represented by the formula (Bn1c12).

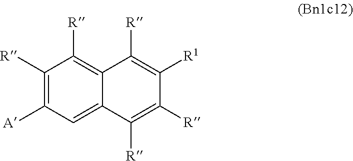

In the formula (Bn1c12), I, R¹, R″, and A are as defined in the formula (Bn1).

The compound represented by the formula (Bn1a) is preferably a compound represented by the formula (Bn1b2).

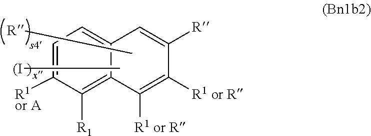

In the formula (Bn1b2), I, R¹, R″, A, and nd are as defined in the formula (Bn1). x″ is 0 or 1. s4′ is defined as mentioned above.

The compound represented by the formula (Bn1b2) is preferably a compound represented by the formula (Bn1c21).

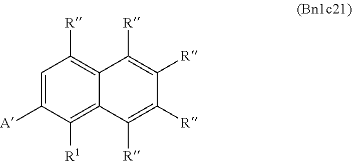

In the formula (Bn1c21), I, R¹, R″, A, and nd are as defined in the formula (Bn1).

The compound represented by the formula (Bn1a) is preferably a compound represented by the formula (Bn1b3).

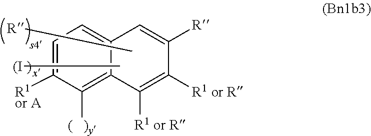

In the formula (Bn1b3), I, R¹, R″, A, and nd are as defined in the formula (Bn1). Each of x′ and y′ is 0 or 1, and x′ and y′ satisfy (x′+y′)≤(x+y−1), with respect to x and y in the formula (1n). s4′ is defined as mentioned above.

The compound represented by the formula (Bn1b3) is preferably a compound represented by the formula (Bn1c31).

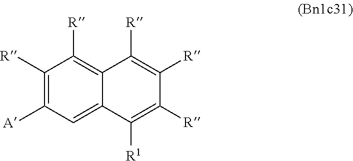

In the formula (Bn1c31), I, R¹, R″, A, and nd are as defined in the formula (Bn1).

The compound represented by the formula (Bn1b3) is preferably a compound represented by the following formula (Bn1c32).

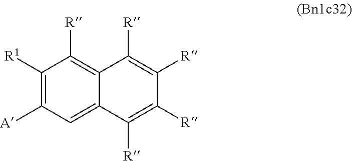

In the formula (Bn1c22), I, R¹, R″, A, and nd are as defined in the formula (Bn1).

The compound represented by the formula (Ba1) is preferably a compound represented by the formula (Ba1a).

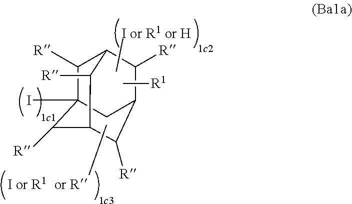

In the formula (Ba1a), I, R, R¹, A, Z, and Rd are as defined in the formula (Ba1) 1c1, 1c2, and 1c3 are an integer of 0 or 1 satisfying (1c1+1c2+1c3) t1b. Although t1b is defined as mentioned above, t1b preferably has the same meaning as t1b in the formula (Ba1) (the same applies hereinafter).

The compound represented by the formula (Ba1a) is preferably a compound represented by the formula (Ba1b).

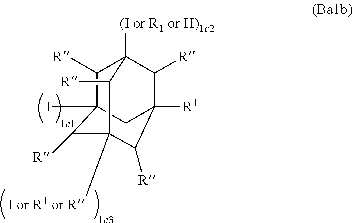

In the formula (Ba1b), I, R, R¹, A, and Z are as defined in the formula (Ba1a) 1c1, 1c2, and 1c3 are an integer of 0 or 1 satisfying (1c1+1c2+1c3)≤t1b.

The compound represented by the formula (Ba1b) is preferably a compound represented by the following formula (Ba1c11).

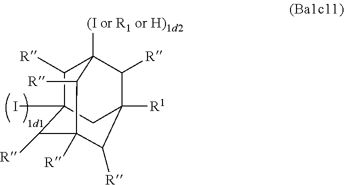

In the formula (Ba1c11), I, R″, and R′ are as defined in the formula (Ba1a). 1d1 and 1d2 are an integer of 0 or 1 satisfying (1d1+1d2)≤t1b.

The compound represented by the formula (Ba1b) is preferably a compound represented by the following formula (Ba1c12).

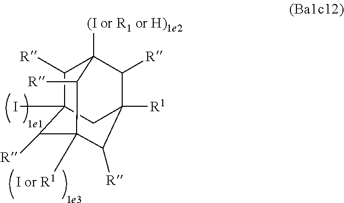

In the formula (Ba1c12), I, R, and R¹ are as defined in the formula (Ba1a), and 1e1, 1e2, and 1e3 are an integer of 0 or 1 satisfying (1e1+1e2+1e3)≤t1b.

### [Base Material (A)]

In the present embodiment, the base material (A) refers to a material other than the compound (B), which can be used as a resist. The base material (A) may be a resin. For example, the base material (A) refers to a base material that can be used as a resist for g-ray, i-ray, KrF excimer laser (248 nm), ArF excimer laser (193 nm), extreme ultraviolet (EUV) lithography (13.5 nm) or electron beam (EB) (for example, a base material for lithography or a base material for resist). Examples of the base material (A) include a phenol novolac resin, a cresol novolac resin, a hydroxystyrene resin, a (meth)acrylic resin, a hydroxystyrene-(meth)acrylic copolymer, a cycloolefin-maleic anhydride copolymer, a cycloolefin, a vinyl ether-maleic anhydride copolymer, an inorganic resist material having a metallic element such as titanium, tin, hafnium and zirconium, and derivatives thereof. Among them, from the viewpoint of the shape of a resist pattern to be obtained, preferable are a phenol novolac resin, a cresol novolac resin, a hydroxystyrene resin, a (meth)acrylic resin, a hydroxystyrene-(meth)acrylic copolymer, and an inorganic resist material having a metallic element such as titanium, tin, hafnium, and zirconium, and derivatives thereof.

The weight average molecular weight of the base material (A) is preferably 2000 to 49900, more preferably 2000 to 29900, and further preferably 2000 to 14900, from the viewpoints of reducing defects in a film to be formed by using the composition and of a good pattern shape. As the above weight average molecular weight, a value obtained by measuring the weight average molecular weight in terms of polystyrene, using GPC, can be used.

### [Solvent (S)]

As the solvent in the present embodiment, a known solvent can be arbitrarily used as long as it can at least dissolve the compound (B). Specific examples of the solvent include, ethylene glycol monoalkyl ether acetates; ethylene glycol monoalkyl ethers; propylene glycol monoalkyl ether acetates (e.g., propylene glycol monomethyl ether acetate); propylene glycol monoalkyl ethers; lactate esters; aliphatic carboxylic acid esters; other esters; aromatic hydrocarbons; ketones; amides 3:9; and lactones. Specific examples thereof include those disclosed in Patent Literature 1.

The solvent used in the present embodiment is preferably a safe solvent, more preferably at least one selected from propylene glycol monomethyl ether acetate (PGMEA), propylene glycol monomethyl ether (PGME), cyclohexanone (CHN), cyclopentanone (CPN), 2-heptanone, anisole, butyl acetate, and ethyl lactate, and further preferably at least one selected from PGMEA, PGME, CHN, CPN, and ethyl lactate.

In the present embodiment, the amount of the solid component and the amount of the solvent are not particularly limited, but preferably the solid component is 1 to 80% by mass and the solvent is 20 to 99% by mass, more preferably the solid component is 1 to 50% by mass and the solvent is 50 to 99% by mass, further preferably the solid component is 2 to 40% by mass and the solvent is 60 to 98% by mass, and particularly preferably the solid component is 2 to 10% by mass and the solvent is 90 to 98% by mass, based on the total mass of the amount of the solid component and the solvent. The total mass of the solid component (the summation of the solid component including components arbitrarily used such as the base material (A), the compound (B), the acid generating agent (C), the crosslinking agent (G), the acid diffusion controlling agent (E) and the further component (F), and the same applies hereinafter) is set to the amount of the solid component.

### [Acid Generating Agent (C)]

The composition of the present embodiment preferably contains one or more acid generating agents (C). The acid generating agent (C) is a material generating an acid directly or indirectly by irradiation of any radiation selected from visible light, ultraviolet, excimer laser, electron beam, extreme ultraviolet (EUV), X-ray and ion beam. As the acid generating agent (C), for example, those described in International Publication No. WO 2013/024778 can be used. Two or more kinds of acid generating agents (C) can also be used in combination.

The amount of the acid generating agent (C) used is preferably 0.001 to 49% by mass of the total mass of the solid component, more preferably 1 to 40% by mass, still more preferably 3 to 30% by mass, and particularly preferably 10 to 25% by mass. By using the acid generating agent (C) within the above range, there is a tendency that a pattern profile with high sensitivity and low edge roughness is obtained.

### [Crosslinking Agent (G)]

The composition of the present embodiment preferably contains one or more crosslinking agents (G). The cross-linking agent (G) may crosslink at least the base material (A) or the compound (B). The crosslinking agent (G) intramolecularly or intermolecularly crosslinks the base material (A) in the presence of the acid generated from the acid generating agent (C). Examples of such an acid crosslinking agent can include a compound having one or more groups capable of crosslinking the base material (A) (hereinafter, referred to as a “crosslinkable group”). As the crosslinking agent (G) having the crosslinkable group, for example, a crosslinking agent described in International Publication No. WO 2013/024778 can be used. Two or more kinds of crosslinking agents (G) can also be used in combination.

In the present embodiment, the amount of the crosslinking agent (G) used is preferably 0.5 to 50% by mass of the total mass of the solid component, more preferably 0.5 to 40% by mass, still more preferably 1 to 30% by mass, and particularly preferably 2 to 20% by mass. When the content ratio of the crosslinking agent (G) used is 0.5% by mass or more, there is a tendency that the inhibiting effect of the solubility of a resist film in an alkaline developing solution is improved and that a decrease in the film remaining rate, as well as occurrence of swelling and meandering of a pattern, can be inhibited. On the other hand, when the content ratio is 50% by mass or less, there is a tendency that a decrease in heat resistance as a resist can be inhibited.

### [Acid Diffusion Controlling Agent (E)]

The composition of the present embodiment may contain the acid diffusion controlling agent (E). The acid diffusion controlling agent (E) has effects of controlling diffusion of an acid generated from an acid generating agent by radiation irradiation in a resist film, inhibiting any unpreferable chemical reaction in an unexposed region, and the like. By using the acid diffusion controlling agent (E), there is a tendency that the storage stability of the composition of the present embodiment can be improved. By using the acid diffusion controlling agent (E), the resolution of the film formed by using the composition of the present embodiment can be improved. In addition, by using the acid diffusion controlling agent (E), there is a tendency that the line width change of a resist pattern due to variation in the post exposure delay time before radiation irradiation and the post exposure delay time after radiation irradiation can be suppressed, and the process stability is improved. Examples of the acid diffusion controlling agent (E) include radiation degradable basic compounds described in International Publication No. WO 2013/024778. Two or more kinds of acid diffusion controlling agents (E) can also be used in combination.

The content of the acid diffusion controlling agent (E) is preferably 0.001 to 49% by mass of the total mass of the solid component, more preferably 0.01 to 10% by mass, still more preferably 0.01 to 5% by mass, and particularly preferably 0.01 to 3% by mass. When the amount of the acid diffusion controlling agent (E) blended is within the above range, there is a tendency that a decrease in resolution, and deterioration of the pattern shape and the dimension fidelity or the like can be prevented. Moreover, even though the post exposure delay time from electron beam irradiation to heating after radiation irradiation becomes longer, the shape of the pattern upper layer portion can be prevented from being deteriorated. Also, when the content is 10% by mass or less, there is a tendency that a decrease in sensitivity, and developability of the unexposed portion or the like can be prevented. Also, by using such an acid diffusion controlling agent, there is a tendency that the storage stability of a resist composition is improved, also along with improvement of the resolution, the line width change of a resist pattern due to variation in the post exposure delay time before radiation irradiation and the post exposure delay time after radiation irradiation can be suppressed, and process stability is improved.

### [Further Component (F)]

The composition of the present embodiment can contain one or more of the following additives as the further component (F).

### (Dissolution Promoting Agent)

When the solubility of a solid component is too low, the dissolution promoting agent increases the solubility of the solid component in a developing solution to moderately increase the dissolution rate of the compound upon developing. As the above dissolution promoting agent, those having a low molecular weight are preferable, and examples thereof can include a phenolic compound having a low molecular weight. Examples of the phenolic compound having a low molecular weight can include a bisphenol and a tris(hydroxyphenyl)methane. Two or more kinds of dissolution promoting agents can also be used in combination.

The amount of the dissolution promoting agent blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Dissolution Controlling Agent)

When the solubility of a solid component is too high, the dissolution controlling agent controls the solubility of the solid component in a developing solution to moderately decrease the dissolution rate upon developing. As such a dissolution controlling agent, the one which does not chemically change in steps such as calcination of resist coating, radiation irradiation, and development is preferable.

The dissolution controlling agent is not particularly limited, and examples thereof include an aromatic hydrocarbon such as phenanthrene, anthracene and acenaphthene; a ketone such as acetophenone, benzophenone and phenyl naphthyl ketone; and a sulfone such as methyl phenyl sulfone, diphenyl sulfone and dinaphthyl sulfone. Two or more kinds of dissolution controlling agents can also be used in combination. The amount of the dissolution controlling agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Sensitizing Agent)

The sensitizing agent absorbs irradiated radiation energy, transmits the energy to the acid generating agent (C), and thereby increases the acid production amount, and improves the apparent sensitivity of a resist. Examples of such a sensitizing agent include benzophenones, biacetyls, pyrenes, phenothiazines, and fluorenes. Two or more kinds of sensitizing agents can also be used in combination. The amount of the sensitizing agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Surfactant)

The surfactant improves coatability and striation of the composition of the present embodiment, developability of a resist, and the like. The surfactant may be an anionic surfactant, a cationic surfactant, a nonionic surfactant, or an amphoteric surfactant. Preferable examples of the surfactant include a nonionic surfactant. The nonionic surfactant has a good affinity with a solvent to be used in production of the composition of the present embodiment, and can further enhance the effects of the composition of the present embodiment. Examples of the nonionic surfactant include, but are not particularly limited to, a polyoxyethylene higher alkyl ether, a polyoxyethylene higher alkyl phenyl ether, and a higher fatty acid diester of polyethylene glycol. Commercial products described in Patent Literature 1 as these surfactants can also be used. The amount of the surfactant blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Organic Carboxylic Acid, or Oxo Acid of Phosphorus or Derivative of Oxo Acid)

The organic carboxylic acid, or the oxo acid of phosphorus or the derivative of the oxo acid (hereinafter, also referred to as “acid or derivatives”) has functions of preventing sensitivity deterioration, improving a resist pattern shape, improving exposure delay stability, and the like. Examples of the organic carboxylic acid include malonic acid as described in Patent Literature 1. Examples of the oxo acid of phosphorus or the derivative thereof include derivatives such as phosphonic acid or an ester thereof described in Patent Literature 1, and among them, phosphonic acid is particularly preferable.

The acid or the derivative can be used alone or in combination of two or more kinds. The amount of the acid or the derivative blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Other Additives)

Furthermore, the composition of the present embodiment can contain an additive other than the components mentioned above, if required. Examples of such an additive include a dye, a pigment and an adhesion aid. For example, when the composition is blended with a dye or a pigment, a latent image of the exposed portion is visualized and influence of halation upon exposure can be alleviated, which is preferable. Also, when the composition is blended with an adhesion aid, adhesiveness to a substrate can be improved, which is preferable. Furthermore, examples of the other additives include a halation preventing agent, a storage stabilizing agent, a defoaming agent and a shape improving agent. Specific examples thereof include 4-hydroxy-4′-methylchalkone.

### [Content Ratio of Each Component in Composition]

In the composition of the present embodiment, the amount of the compound B is preferably 10 ppm to 10% by mass in the total mass of the solid component of the composition. In the present disclosure, the total mass of the solid component is the summation of the solid component including components arbitrarily used such as the base material (A), the compound (B), the acid generating agent (C), the crosslinking agent (G), the acid diffusion controlling agent (E) and the further component (F). The mass ratio of the base material (A) to the compound (B) is preferably 3:97 to 99.5:0.5, and more preferably 10:90 to 99:1. When the mass ratio falls within this range, the composition tends to have high sensitivity and suppressed exposure dispersion in the depth direction. The mass ratio is more preferably 30:70 to 98:2, and further preferably 50:50 to 97:3.

In the composition of the present embodiment, the total amount of the base material (A) and the compound (B) is preferably 50 to 99.4% by mass of the total mass of the solid component, more preferably 55 to 95% by mass, further preferably 60 to 95% by mass, and particularly preferably 70 to 95% by mass. When the total amount of the base material (A) and the compound (B) is the above the content, there is a tendency that resolution is further improved and that line edge roughness (LER) is further decreased.

In the composition of the present embodiment, the (A)/(B)/(C)/(G)/(E)/(F) mass ratio (% by mass) is, based on the total mass of the solid content of the composition of the present embodiment:
preferably 1.5 to 99.0/0.2 to 96.4/0.001 to 49/0 to 49/0.001 to 49/0 to 49,
more preferably 5 to 98.5/0.5 to 89/1 to 40/0 to 40/0.01 to 10/0 to 5,
further preferably 15 to 97.5/1 to 69/3 to 30/0 to 30/0.01 to 5/0 to 1, and
particularly preferably 25 to 96.5/1.5 to 50/3 to 30/0 to 30/0.01 to 3/0.

The content ratio of each component is selected from each range so that the summation thereof is 100% by mass. Through the content ratio, there is a tendency that performance such as sensitivity, resolution and developability is excellent. A “solid content” refers to a component except for the solvent. The “total mass of the solid content” means that the total of components excluding the solvent from the components constituting the composition is 100% by mass.

The composition of the present embodiment is normally prepared by dissolving each component in a solvent upon use into a homogeneous solution, and then if required, filtering through a filter or the like with a pore diameter of about 0.2 μm, for example.

### [Physical Properties and the Like of Composition]

The composition of the present embodiment can form an amorphous film by spin coating. Also, the composition of the present embodiment can be applied to a general semiconductor production process. In addition, any of positive type or negative type resist patterns can be individually prepared from the composition of the present embodiment, depending on the kind of a developing solution to be used.

A composition for lithography containing the compound (B) exhibits an excellent sensitizing effect in EUV exposure. Thus, the present invention also provides a method for enhancing the sensitivity of the composition for lithography in EUV exposure. As mentioned above, two or more kinds of the compounds (B) are preferably used in the sensitizing method.

The residual amounts of metal impurities in the composition are preferably less than 1 ppm, more preferably less than 100 ppb, further preferably less than 50 ppb, still more preferably less than 10 ppb, and most preferably less than 1 ppb, based on the composition. In particular, with respect to metal species such as Fe, Ni, Sn, Zn, Cu, Sb, W, and Al which are classified as transition metals, there is concern that the residual amounts of metals of 1 ppm or more may cause the denaturation and deterioration of materials with time due to the interaction with other compounds. When the residual amounts of alkaline metals or alkaline earth metals such as Na, K, Ca, and Mg are 1 ppm or more, there is a concern that the metal balance cannot be sufficiently reduced in the preparation of a resin for semiconductor process using the compound, which causes defects derived from residual metals in a semiconductor production process and reduction in the yield due to performance deterioration.

### EXAMPLES

### [Example 1] Compound Having Benzene Ring as Core

The compound was produced according to the following scheme. The reaction was conducted under a nitrogen stream.

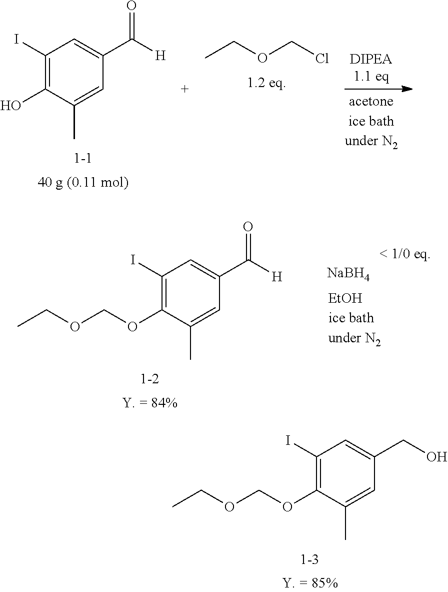

### (Synthesis of Compound 1-2)

A flask equipped with a stirrer and a condenser was immersed in an ice bath, and the flask was charged with 40 g (0.11 mol) of the compound 1-1 (manufactured by Tokyo Kasei Kogyo Co., Ltd.) and 120 mL of acetone and stirred. At this time, the internal temperature was 4° C. Then, 15.2 g (1.1 equivalents to the compound 1-1) of diisopropylethylamine (DIPEA) was added dropwise into the flask. After completion of the dropwise addition of diisopropylethylamine, 12.3 g (1.2 equivalents to the compound 1-1) of chloromethyl ethyl ether was added dropwise, and the reaction was carried out under a nitrogen stream for 2 hours. After completion of the reaction, 120 mL of water was added into the flask to obtain a precipitate. Subsequently, the precipitate was filtered and washed with water, further washed with methanol, and then filtered and dried to obtain the compound 1-2. The yield was 84%.

### (Synthesis of Compound 1-3)

A flask equipped with a stirrer and a condenser was immersed in an ice bath, and the flask was charged with 33 g of the compound 1-2 and 100 mL of dehydrated ethanol. At this time, the internal temperature was 3° C. Then, 2.88 g (less than 1.0 equivalent to the compound 1-2) of NaBH₄ was added portionwise over 1 hour. Thereafter, the reaction was continued under a nitrogen stream for 45 minutes. Subsequently, 78 g of 5% ammonium chloride (a reagent manufactured by FUJIFILM Wako Pure Chemical Corporation) and 100 g of water were added, and the mixture was filtered, washed with water, and dried to obtain the compound 1-3. The yield was 85%.

### [Example 1a] Compound 2 Having Benzene Ring as Core

60 ml of acetone was added to 16 g (65 mmol) of 5-iodovanillin, and the mixture was ice cooled. After 8.2 g (63 mmol) of diisopropylethylamine was added under nitrogen, 6.4 ml (0.84 mol) of chloromethyl ethyl ether was added dropwise at 12° C. or less. The mixture was stirred at 3° C. for 15 minutes, and 100 ml of water was slowly added. The precipitate was collected by filtration and washed with water. The obtained solid was suspended with 70 ml of methanol, stirred, and filtered. The solid was dried at room temperature, and used in the next step. 15 ml of ethanol was added to 2 g of the solid, and the mixture was ice cooled. 225 mg (5.9 mmol) of sodium borohydride was added portionwise over 5 minutes. The mixture was reacted for 30 minutes as it is, and 20 ml of water was added. After 400 mg (7.5 mmol) of ammonium chloride was added, 20 ml of water was added dropwise. After ethyl acetate was added for extraction, the extract was dried over sodium sulfate, and the organic solvent was distilled off using an evaporator to obtain the compound 2.

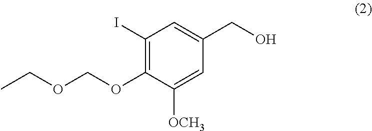 [SMILES: CCOCOc1c(I)cc(CO)cc1OC]

### [Example 1b] Compound 3 Having Benzene Ring as Core

The compound 3 was obtained in the same manner as in Example 1, except for using 9.38 g of ethyl vinyl ether instead of 12.3 g of chloromethyl ethyl ether.

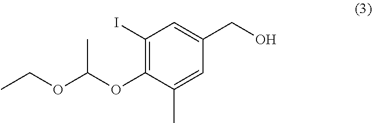 [SMILES: CCOC(C)Oc1c(C)cc(CO)cc1I]

### [Example 1c] Compound 4 Having Benzene Ring as Core

The compound 4 was obtained in the same manner as in Example 1, except for using 11.2 g of tetrahydropyran instead of 12.3 g of chloromethyl ethyl ether.

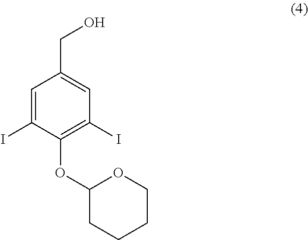 [SMILES: OCc1cc(I)c(OC2CCCCO2)c(I)c1]

### [Example 1d] Compound 5 Having Benzene Ring as Core

The compound 5 was obtained in the same manner as in Example 1, except for using 14.2 g of di-tert-butyl dicarbonate instead of 12.3 g of chloromethyl ethyl ether.

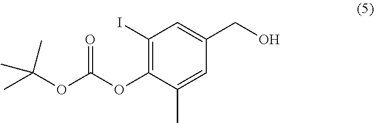 [SMILES: Cc1cc(CO)cc(I)c1OC(=O)OC(C)(C)C]

### [Example 1e] Compound 6 Having Benzene Ring as Core

3,5-Diiodo-4-hydroxybenzyl alcohol was obtained in the same manner as in Example 1. After 3,5-diiodo-4-hydroxybenzyl alcohol and THF were added, stirred, and dissolved, phosgene (2 equivalents to the raw material, a 20% toluene solution, manufactured by Merck) was added dropwise in a nitrogen atmosphere under ice cooling, and the mixture was further stirred under ice cooling for 2 hours. Further, the mixture was stirred at 25° C. for 12 hours. Thereafter, the mixture was subjected to nitrogen bubbling for 2 hours, and concentrated under reduced pressure to obtain a carboxylate (1e0). The obtained carboxylate (1e0) was put in chloroform, and the mixture was stirred under ice cooling for dissolution. Further, 1-methylcyclopentanol (1.2 equivalents to (1e0)) was added dropwise under ice cooling and stirred. Further, pyridine (1.2 equivalents to (1e0)) was added dropwise under ice cooling and stirred. The mixture was stirred for 1 hour, and then stirred at 25° C. for 12 hours. Thereafter, ion-exchange water was added, and then, the organic phase was recovered. The obtained organic phase was washed with a 5% sodium bicarbonate solution, and then subjected to washing treatment with ion-exchange water 5 times, and concentrated under reduced pressure to obtain the compound (6).

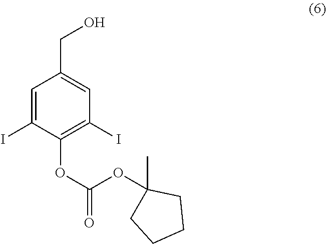 [SMILES: CC1(OC(=O)Oc2c(I)cc(CO)cc2I)CCCC1]

### [Example 1f] Compound 7 Having Benzene Ring as Core

The compound 7 was obtained in the same manner as in Example 1, except for using 10.5 g of chloromethyl methyl ether instead of 12.3 g of chloromethyl ethyl ether.

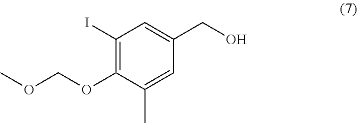

### Synthesis Example L1

### [Iodination Step]

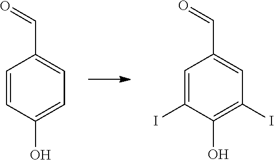 [SMILES: O=Cc1ccc(O)cc1 ; O=Cc1cc(I)c(O)c(I)c1]

A 100 L glass lined reaction vessel connected with a reflux tube was charged with 700 g of 4-hydroxybenzaldehyde, 4900 ml of methanol, and 1260 mL of pure water, and the mixture was stirred under nitrogen flow at 220 rpm for 1 hour for dissolution. Further, 1590 g of sodium bicarbonate was gradually added in 10 portions for 10 minutes, and then 3200 g of iodine was gradually added in 10 portions for 40 minutes. At this time, the liquid temperature was increased to 47° C., and bubbling was observed. The liquid was stirred for 8 hours in a state where the internal temperature was maintained at 46° C. in a hot water bath. At the timepoints of 5 hours after stirring, 300 g of iodine was additionally added. After that, the liquid was stirred for 24 hours under the conditions of 24° C. and 70 rpm. Thereafter, 21 L of an aqueous solution of 6 M hydrochloric acid was added dropwise at 120 rpm over 1 hour, and then, the mixture was stirred for 30 minutes. Then, 2.3 L of an aqueous solution of 20 wt. % sodium sulfite was added while stirring, and then 3.5 L of pure water was further added, and the formed precipitate was filtered off by filtration and recovered. Further, the recovered product was subjected to rinsing treatment using 2 L of methanol, and then dried to obtain 1880 g of 4-hydroxy-3,5-diiodobenzaldehyde in a yield of 87%.

### [Protective Group Introduction Step]

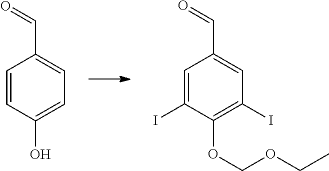

Using a 100 L glass lined reaction vessel connected with a reflux tube, 1822 g of 4-hydroxy-3,5-diiodobenzaldehyde and 3650 mL of dimethylformamide (DMF) subjected to dehydration treatment were put under nitrogen flow in an ice bath state, and the mixture was stirred with a stirring blade for dissolution. Then, 822 g of diisopropylethylamine was added over 30 minutes using a dropping funnel while stirring in an ice bath, and further stirred for 60 minutes. 553 g of chloromethyl ethyl ether (1.2 equivalents to the substrate) was added dropwise to the stirred reaction solution over 60 minutes using a dropping funnel, and further stirred for 30 minutes in an ice bath. Thereafter, 7.2 L of pure water was added thereto in an ice bath, the mixture was stirred for 60 minutes, and then the formed precipitate was recovered by filtration. The recovered solid was suspended in 5.8 L of methanol in an ice bath and stirred for 30 minutes, and then 2450 g of white solid was obtained by filtration as a protected compound which is the target substance. The yield was 99%.

### [Reduction Step]

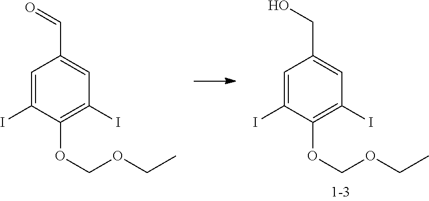

A 20 L separable flask was filled with ethanol (5 L) in an ice bath, and 2450 g of the protected compound obtained in the former step was gradually put for suspension. 50 g of sodium borohydride was added portionwise in 5 g portions over 60 minutes while stirring under nitrogen flow. Further, after the mixture was stirred in an ice bath for 1 hour, 842 g of a 5% by mass ammonium chloride solution was added dropwise over 15 minutes. The obtained reaction solution was gradually added to 21 L of pure water under ice cooling and stirred for 30 minutes. The precipitate gradually formed during stirring was filtered off, and then further subjected to rinsing treatment with 5 L of pure water. The obtained precipitate was dissolved in 10.5 L of ethyl acetate, and then subjected to washing treatment using 3.5 L of an aqueous 10% by mass NaCl solution three times, the obtained ethyl acetate solution was recovered, and then, 200 g of magnesium sulfate was further added and suspended for 30 minutes. The filtrate obtained by filtration was concentrated to a concentration of about 50% by mass±5%, and 9 L of heptane was further put for crystallization. The crystallized substance filtered off was further subjected to rinsing treatment with cold heptane and then dried to obtain 1680 g of the target substance (1-3) in a yield of 77% with a purity of 99.8%.

### (Synthesis Example L2)

The [iodination step], [protective group introduction step], and [reduction step] were conducted in the same manner as in Synthesis Example L1, except that salicylaldehyde was used instead of 4-hydroxybenzaldehyde and the iodination step was changed to the following iodination step L2, thereby obtaining a target substance (1-4).

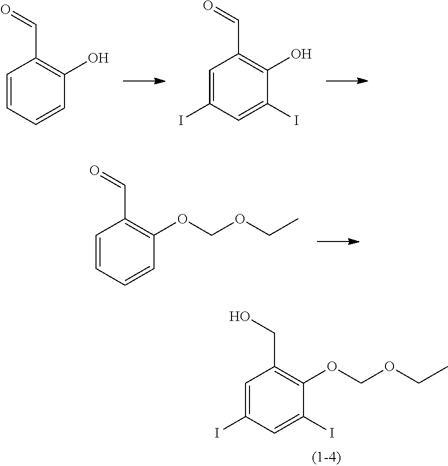

### [Iodination Step L2]

A 100 L glass lined reaction vessel connected with a reflux tube was charged with 700 g of salicylaldehyde, 5700 ml of ethanol, and 1164 g of iodine, and the mixture was heated under nitrogen flow with a water bath so that the internal temperature reached 40° C., and stirred at 220 rpm for 1 hour for dissolution. Further, 2490 g of an aqueous solution of 20% by mass iodic acid was slowly added dropwise over 60 minutes. Thereafter, the internal temperature was increased to 50° C., and the mixture was continuously stirred for 2 hours. Then, 2.3 L of an aqueous solution of 20 wt. % sodium sulfite was added while stirring, and then 7.6 L of pure water was further added, and the formed precipitate was filtered off by filtration and recovered. Further, the recovered product was subjected to rinsing treatment using 5 L of pure water, and then dried to obtain 2046 g of 2-hydroxy-3,5-diiodobenzaldehyde in a yield of 95.5%.

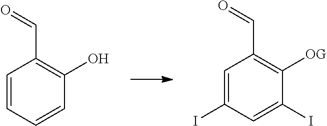

### Synthesis Example L3

The [iodination step], [protective group introduction step], and [reduction step] were conducted in the same manner as in Synthesis Example L1, except that vanillin(4-hydroxy-3-methoxybenzaldehyde) was used instead of 4-hydroxybenzaldehyde and the iodination step was changed to the following [iodination step L3], thereby obtaining a target compound (1-5).

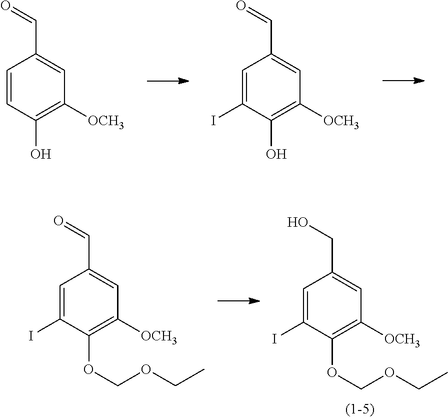 [SMILES: COc1cc(C=O)ccc1O ; COc1cc(C=O)cc(I)c1O ; CCOCOc1c(I)cc(C=O)cc1OC ; CCOCOc1c(I)cc(CO)cc1OC]

### [Iodination Step L3]

A 30 L glass reaction vessel was charged with 1300 g (8.55 mol) of vanillin(4-hydroxy-3-methoxybenzaldehyde) and 5.6 L of methanol, and blowing of nitrogen and stirring into the reaction vessel at a flow rate of 200 mL/min were initiated. After dissolution of vanillin was confirmed, the reaction vessel was charged with 2.6 L of ion-exchange water and 635 g (6 mol) of sodium carbonate, and the mixture was stirred at a room temperature of 22° C. for 3 hours. The reaction vessel was charged with 2600 g (10.3 mol) of iodine by portionwise addition and stirred at a room temperature of 22° C. for 20 hours, and then, a 16.6% aqueous sodium sulfite solution was added until it was confirmed that the solution was decolorized and the system reached a basic system. 4.3 L of water was added, and the mixture was stirred for 1 hour. The precipitated solid was filtered with a suction filter, rinsed, reslurried, and dried to obtain 1900 g of a white solid. As a result of the analysis by liquid chromatography-mass spectrometry (LC-MS), the molecular weight was found to be 278. The ¹H-NMR measurement was carried out under the above measurement conditions, and the compound was confirmed to have the chemical structure of 4-hydroxy-5-iodo-3-methoxybenzaldehyde. The LC purity at 254 nm was 99.8%, and the GPC purity was 99.9%.

### Synthesis Example L4

The [iodination step], [protective group introduction step], and [reduction step] were conducted in the same manner as in Synthesis Example L3, except that 1420 g of ethylvanillin was used instead of 1300 g of vanillin, thereby obtaining a target substance (1-6).

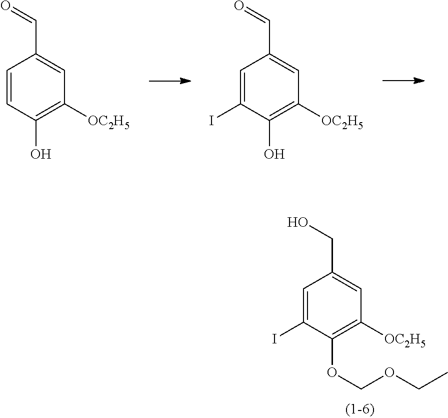 [SMILES: CCOc1cc(C=O)ccc1O ; CCOc1cc(C=O)cc(I)c1O ; CCOCOc1c(I)cc(CO)cc1OCC]

### Synthesis Example L5

The [iodination step], [protective group introduction step], and [reduction step] were conducted in the same manner as in Synthesis Example Li, except that 3-hydroxybenzaldehyde was used instead of 4-hydroxybenzaldehyde, thereby obtaining a target substance (1-7).

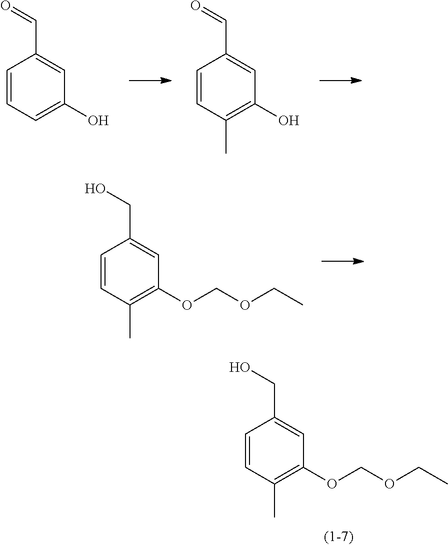

### Synthesis Example L6

The protective group introduction step of Synthesis Example L3 was carried out by using 3,4-dihydroxybenzaldehyde as the raw material. Provided that, the amounts of diisopropylmethylamine and chloromethyl ethyl ether to 3,4-dihydroxybenzaldehyde were set to 2 times. Thereafter, the following [iodination step L6] was conducted. Then, [the reduction step] was conducted in the same manner as in Synthesis Example L3 to obtain a target substance (1-8).

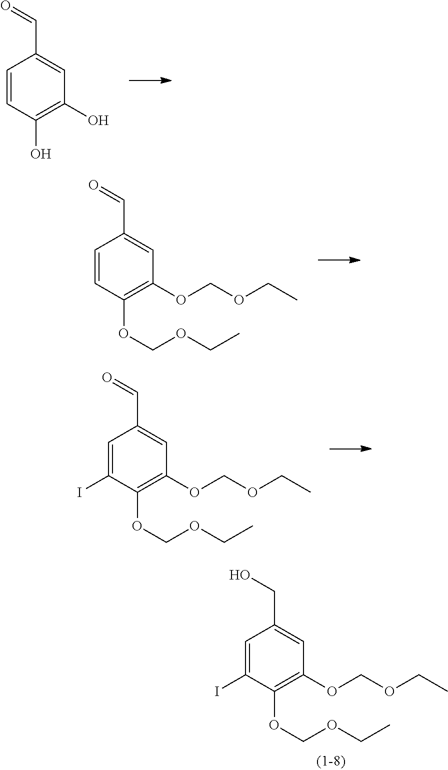

### [Iodination Step L6]

A 30 L glass reaction vessel was charged with 2172 g (8.55 mol) of 3,4-diethoxymethoxybenzaldehyde as a raw material and 5.6 L of methanol, and blowing of nitrogen into the reaction vessel at a flow rate of 200 mL/min and stirring were initiated. After dissolution of the raw material was confirmed, the reaction vessel was charged with 2.6 L of ion-exchange water and 634 g (5.99 mol) of sodium carbonate, and the mixture was stirred at a room temperature of 22° C. for 3 hours. Then, the reaction vessel was charged with 2609 g (10.3 mol) of iodine and stirred at a room temperature of 22° C. for 12 hours, and then, a 16.6% aqueous sodium sulfite solution was added until the solution was decolorized, and 4.3 L of water was added and stirred for 1 hour. The precipitated solid was filtered with a suction filter, rinsed, reslurried, and dried to obtain 2438 g of a white solid. As a result of the analysis by liquid chromatography-mass spectrometry (LC-MS), the molecular weight was found to be 380. The ¹H-NMR measurement was carried out under the above measurement conditions, and the compound was confirmed to have the chemical structure of 3,4-diethoxymethoxy-5-iodobenzaldehyde. The LC purity at 254 nm was 99.5%, and the GPC purity was 99.9%.

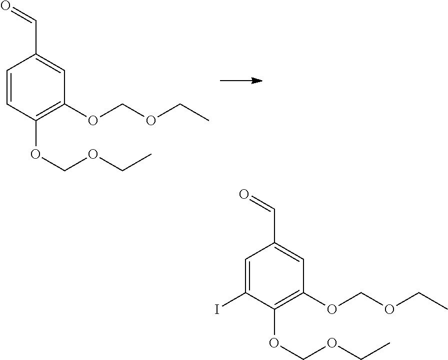 [SMILES: CCOCOc1ccc(C=O)cc1OCOCC ; CCOCOc1cc(C=O)cc(I)c1OCOCC]

### Synthesis Example BPL1

### [Protective Group Introduction Step BPL1]

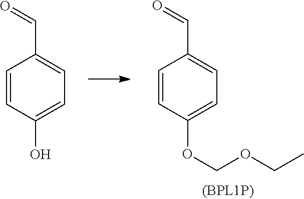

Using a 100 L glass lined reaction vessel connected with a reflux tube, 700 g of 4-hydroxybenzaldehyde and 3650 mL of dimethylformamide (DMF) subjected to dehydration treatment were put under nitrogen flow in an ice bath state, and the mixture was stirred with a stirring blade for dissolution. Then, 822 g of diisopropylethylamine was added over 30 minutes using a dropping funnel while stirring in an ice bath, and further stirred for 60 minutes. 553 g (1.2 equivalents to the substrate) of chloromethyl ethyl ether was added dropwise to the stirred reaction solution over 60 minutes using a dropping funnel, and further stirred for 30 minutes in an ice bath. Thereafter, 7.2 L of pure water was added thereto in an ice bath, the mixture was stirred for 60 minutes, and then the formed precipitate was recovered by filtration. The recovered solid was suspended in 5.8 L of methanol in an ice bath and stirred for 30 minutes, and then 1012 g of a white solid was obtained by filtration as the protected compound BPL1P which is the target substance. The yield was 98%.

### [Reduction Step BPL1R]

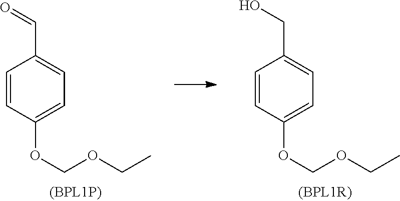

A 20 L separable flask was filled with ethanol (5 L) in an ice bath, and 1012 g of the protected compound BPL1P was gradually put for suspension. 50 g of sodium borohydride was added portionwise in 5 g portions over 60 minutes while stirring under nitrogen flow. Further, after the mixture was stirred in an ice bath for 1 hour, 842 g of a 5% by mass ammonium chloride solution was added dropwise over 15 minutes. The obtained reaction solution was gradually added to 21 L of pure water under ice cooling and stirred for 30 minutes. The precipitate gradually formed during stirring was filtered off, and then further subjected to rinsing treatment with 5 L of pure water. The obtained precipitate was dissolved in 10.5 L of ethyl acetate, and then subjected to washing treatment using 3.5 L of a 10% by mass aqueous NaCl solution three times, the obtained ethyl acetate solution was recovered, and then, 200 g of magnesium sulfate was further added and suspended for 30 minutes. The filtrate obtained by filtration was concentrated to a concentration of about 50% by mass±5%, and 9 L of heptane was further put for crystallization. The crystallized substance filtered off was further subjected to rinsing treatment with cold heptane and then dried to obtain 716 g of the compound BPL1R in a yield of 70% with a purity of 99.6%.

### Synthesis Example BPL1b

THF was added to 4-hydroxybenzylalcohol and stirred for dissolution, and then, phosgene (2 equivalents to the raw material, a 20% toluene solution, manufactured by Merck) was added dropwise in a nitrogen atmosphere under ice cooling, and the mixture was further stirred under ice cooling for 2 hours. Further, the mixture was stirred at 25° C. for 12 hours. Thereafter, the mixture was subjected to nitrogen bubbling for 2 hours, and concentrated under reduced pressure to obtain a carboxylate (1be0). The obtained carboxylate (1be0) was put in chloroform, and the mixture was stirred under ice cooling for dissolution. Further, 1-methylcyclopentanol (1.2 equivalents to (1be0)) was added dropwise under ice cooling and stirred. Further, pyridine (1.2 equivalents to (1be0)) was added dropwise under ice cooling and stirred. The mixture was stirred for 1 hour, and then stirred at 25° C. for 12 hours. Thereafter, ion-exchange water was added, and then, the organic phase was recovered. The obtained organic phase was washed with a 5% sodium bicarbonate solution, and then subjected to washing treatment with ion-exchange water 5 times, and then concentrated under reduced pressure to obtain the compound (BPL1b).

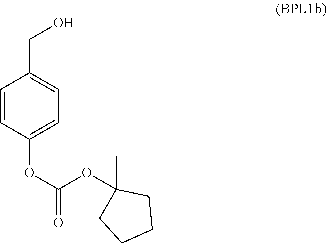 [SMILES: CC1(OC(=O)Oc2ccc(CO)cc2)CCCC1]

### Synthesis Example BPL2

(BPL2P) and (BPL2R) were synthesized in the same manner as in Synthesis Example BPL1-4, except that salicylaldehyde was used as the raw material instead of 4-hydroxybenzaldehyde.

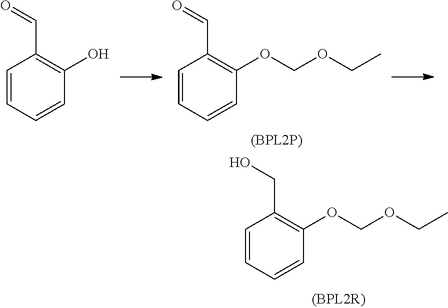

### Synthesis Example BPL3

BPL3P and BPL3R were obtained in the same manner as in Synthesis Example BPL1 except that 3-hydroxybenzaldehyde was used as the raw material.

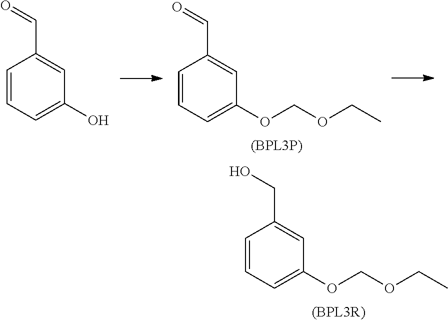

### Synthesis Example DML1

### [Iodination Step DML1D]

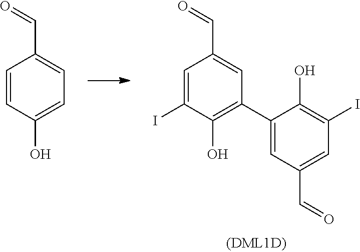 [SMILES: O=Cc1ccc(O)cc1 ; O=Cc1cc(I)c(O)c(-c2cc(C=O)cc(I)c2O)c1]

A 100 L stainless reaction vessel connected with a reflux tube was charged with 700 g of 4-hydroxybenzaldehyde and 4900 ml of methanol, and the mixture was stirred under nitrogen flow at 220 rpm for 1 hour for dissolution. The reaction vessel was ice cooled, an aqueous sodium hydroxide solution prepared by dissolving 757 g of sodium hydroxide in 1260 mL of pure water was gradually added into to the reaction vessel, and then, 3200 g of iodine was gradually added in 10 portions over 60 minutes. The liquid was stirred for 8 hours in a state where the internal temperature was maintained at 60° C. in a hot water bath. Thereafter, 21 L of an aqueous solution of 6 M hydrochloric acid was added dropwise under ice cooling at 120 rpm over 1 hour, and then, the mixture was stirred for 30 minutes. Then, 2.3 L of an aqueous solution of 20 wt. % sodium sulfite was added while stirring, and then 3.5 L of pure water was further added, and the formed precipitate was filtered off by filtration and recovered. The obtained solid was purified by column chromatography using silica gel to obtain 840 g of the compound DML1D in a yield of 30%.

### [Protective Group Introduction Step DML1P]

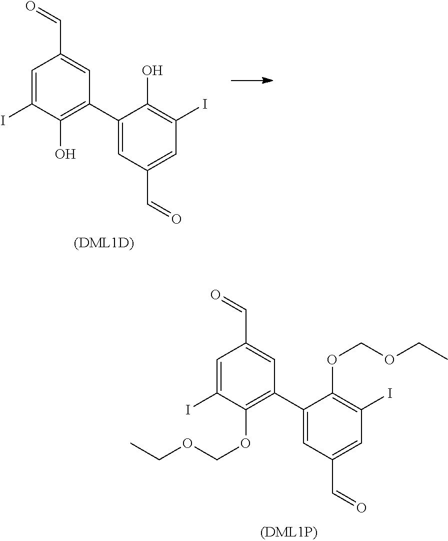 [SMILES: O=Cc1cc(I)c(O)c(-c2cc(C=O)cc(I)c2O)c1 ; CCOCOc1c(I)cc(C=O)cc1-c1cc(C=O)cc(I)c1OCOCC]

Using a 100 L glass lined reaction vessel connected with a reflux tube, 840 g of DML1D and 1680 mL of dimethylformamide (DMF) subjected to dehydration treatment were put under nitrogen flow in an ice bath state, and the mixture was stirred with a stirring blade for dissolution. Then, 380 g of diisopropylethylamine was added over 30 minutes using a dropping funnel while stirring in an ice bath, and further stirred for 60 minutes. 255 g (1.2 equivalents to the substrate) of chloromethyl ethyl ether was added dropwise to the stirred reaction solution over 60 minutes using a dropping funnel, and further stirred for 30 minutes in an ice bath. Thereafter, 7.2 L of pure water was added thereto in an ice bath, the mixture was stirred for 60 minutes, and then, the formed precipitate was recovered by filtration. The recovered solid was suspended in 5.8 L of methanol in an ice bath and stirred for 30 minutes, and then 996 g of a white solid was obtained by filtration as the protected compound DML1P which is the target substance. The yield was 96%.

### [Reduction Step DML1R]

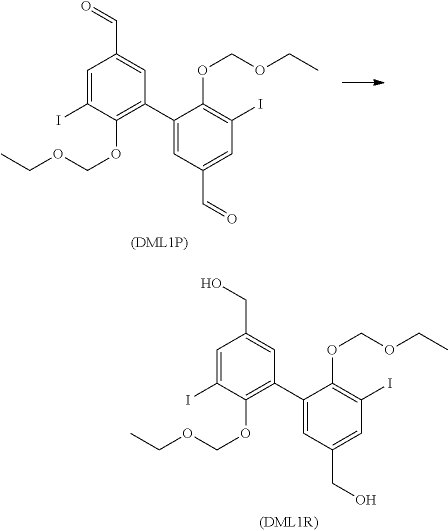

A 20 L separable flask was filled with ethanol (5 L) in an ice bath, and 996 g of the prepared protected compound DML1P was gradually put for suspension. 19 g of sodium borohydride was added portionwise in 3 g portions over 60 minutes while stirring under nitrogen flow. Further, after the mixture was stirred in an ice bath for 1 hour, 350 g of a 5 wt. % ammonium chloride solution was added dropwise over 15 minutes. The obtained reaction solution was gradually added to 8 L of pure water under ice cooling and stirred for 30 minutes. The precipitate gradually formed during stirring was filtered off, and then further subjected to rinsing treatment with 2 L of pure water. The obtained precipitate was dissolved in 4 L of ethyl acetate, and then subjected to washing treatment using 1.5 L of a 10 wt. % aqueous NaCl solution three times, the obtained ethyl acetate solution was recovered, and then, 80 g of magnesium sulfate was further added and suspended for 30 minutes. The filtrate obtained by filtration was concentrated to a concentration of about 50 wt. %±5%, and 9 L of heptane was further put for crystallization. The crystallized substance filtered off was further subjected to rinsing treatment with cold heptane and then dried to obtain 701 g of the compound DML1R in a yield of 70% with a purity of 99.2%.

### Synthesis Example DML2

DML2R was synthesized in the same manner as in Synthesis Example DML1, except that 4-hydroxybenzaldehyde was used as the raw material, the kind of the protecting agent was changed to ethyl vinyl ether, and the [protective group introduction step] was changed to the method described below.

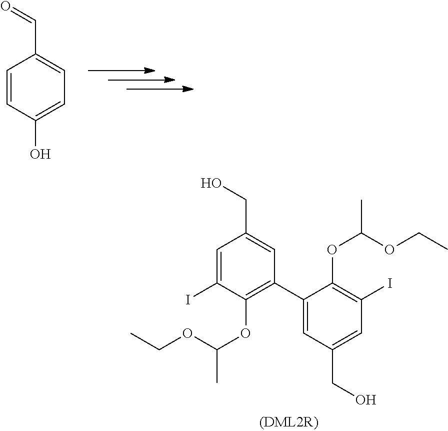 [SMILES: O=Cc1ccc(O)cc1 ; CCOC(C)Oc1c(I)cc(CO)cc1-c1cc(CO)cc(I)c1OC(C)OCC]

### [Protective Group Introduction Step]

Using a 100 L glass lined reaction vessel connected with a reflux tube, 840 g of the compound DML1D and 1680 mL of tetrahydrofuran (THF) subjected to dehydration treatment were put under nitrogen flow in an ice bath state, and the mixture was stirred with a stirring blade for dissolution. Then, 80 g of PPTS (pyridinium-p-toluenesulfonic acid) was added over 30 minutes while stirring in an ice bath, and further stirred for 60 minutes. 294 g (1.2 equivalents to the functional group equivalent) of ethyl vinyl ether was added dropwise to the stirred reaction solution over 60 minutes, and the mixture was further stirred at 35° C. for 60 minutes. Thereafter, 7.2 L of pure water was added thereto in an ice bath, the mixture was stirred for 60 minutes, and then, the organic phase was recovered. To the recovered organic phase, 2 L of ethyl acetate and 5 L of pure water were added and stirred, and then, the organic phase was recovered and concentrated under reduced pressure to obtain 833 g of a white solid as the protected compound DML2P which is the target substance. The yield was 81%.

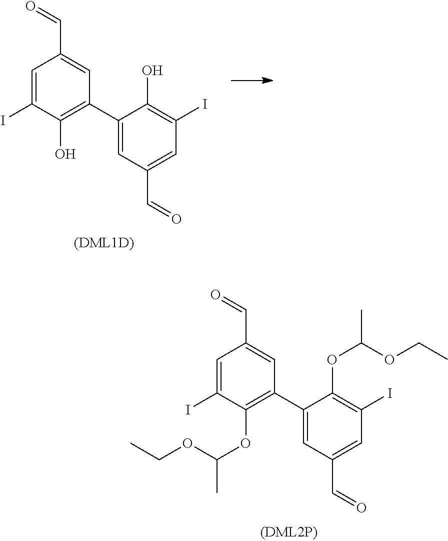 [SMILES: O=Cc1cc(I)c(O)c(-c2cc(C=O)cc(I)c2O)c1 ; CCOC(C)Oc1c(I)cc(C=O)cc1-c1cc(C=O)cc(I)c1OC(C)OCC]

### Synthesis Example DML3

DML3R was synthesized in the same manner as in Synthesis Example DML2, except that 4-hydroxybenzaldehyde was used as the raw material, the kind of the protecting agent was changed to 3,4-dihydropyran, and the [protective group introduction step] was changed to the method described below.

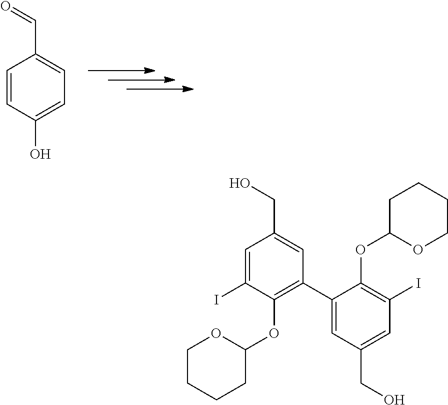 [SMILES: O=Cc1ccc(O)cc1 ; OCc1cc(I)c(OC2CCCCO2)c(-c2cc(CO)cc(I)c2OC2CCCCO2)c1]

### (DML3R)

### Synthesis Example DML4

DML4R was synthesized in the same manner as in Synthesis Example DML1, except that 4-hydroxybenzaldehyde was used as the raw material, the kind of the protecting agent was changed to, and the [protective group introduction step] was changed to the method described below.

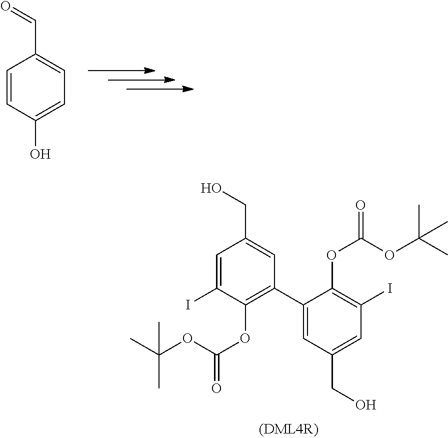 [SMILES: O=Cc1ccc(O)cc1 ; CC(C)(C)OC(=O)Oc1c(I)cc(CO)cc1-c1cc(CO)cc(I)c1OC(=O)OC(C)(C)C]

### [Protective Group Introduction Step]

Using a 100 L glass lined reaction vessel connected with a reflux tube, 840 g of the compound DML1D and 1680 mL of tetrahydrofuran (THF) subjected to dehydration treatment were put under nitrogen flow in an ice bath state, and the mixture was stirred with a stirring blade for dissolution. Then, 10 g (0.05 equivalents to the substrate) of DMAP (dimethylaminopyridine) was added over 10 minutes while stirring in an ice bath, and the mixture was further stirred for 30 minutes. To the stirred reaction solution, 890 g (1.2 equivalents to the functional group equivalent) of di-tert-butyldicarbonate was added dropwise over 60 minutes, and the mixture was further stirred under ice cooling for 120 minutes. After the disappearance of the raw material was confirmed by TLC (thin layer chromatography), 1000 mL of n-heptane was added under cooling, and further, 2800 mL of 1 N hydrochloric acid was added dropwise while confirming the internal temperature of 10° C. or less under ice cooling, and the mixture was further stirred for 30 minutes. The organic phase of the upper layer was recovered, and then subjected to washing treatment with 7 L of a 5% sodium bicarbonate solution (once) and 7 L of ion-exchange water (three times), and then, 50 g of silica gel was put to disperse silica, and the organic phase was recovered by filtration. After MQ (methoquinone) was added in an amount of 200 ppm to the substrate, concentration by displacement with n-heptane (40° C.) were carried out, and the absence of the outflow of THF was confirmed. Thereafter, concentration by displacement was further carried out by using high purity IPA (EL-IPA, Kanto Chemical Co., Inc.). Further, high purity IPA was added at 40° C. for dissolution until the target substance was dissolved, and then, ion-exchange water equivalent to IPA was added dropwise, and crystallization was further carried out under ice cooling for about 1 hour to recover the target substance. 811 g of a white solid was obtained as the protected compound DML4P which is the target substance. The yield was 69%.

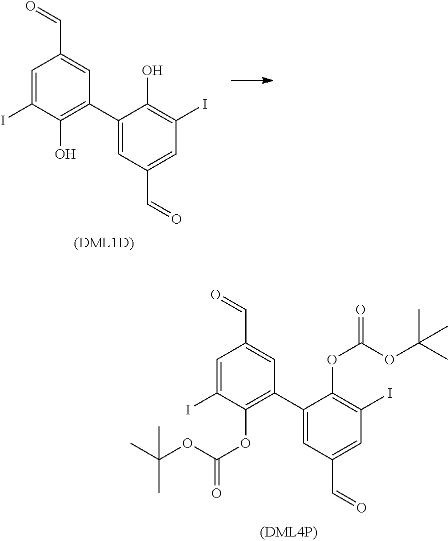 [SMILES: O=Cc1cc(I)c(O)c(-c2cc(C=O)cc(I)c2O)c1 ; CC(C)(C)OC(=O)Oc1c(I)cc(C=O)cc1-c1cc(C=O)cc(I)c1OC(=O)OC(C)(C)C]

### Synthesis Example DML5

DML5R was synthesized in the same manner as in Synthesis Example DML1, except that 3-hydroxybenzaldehyde was used as the raw material.

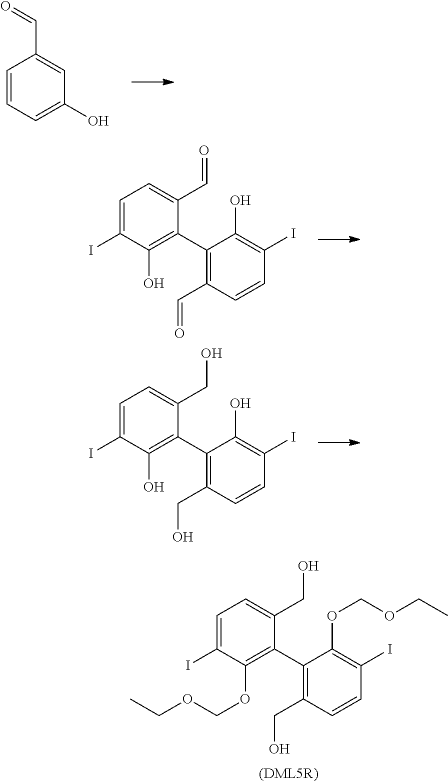

### Synthesis Example DML6

DML6R was synthesized in the same manner as in Synthesis Example DML1, except that vanillin was used as the raw material.

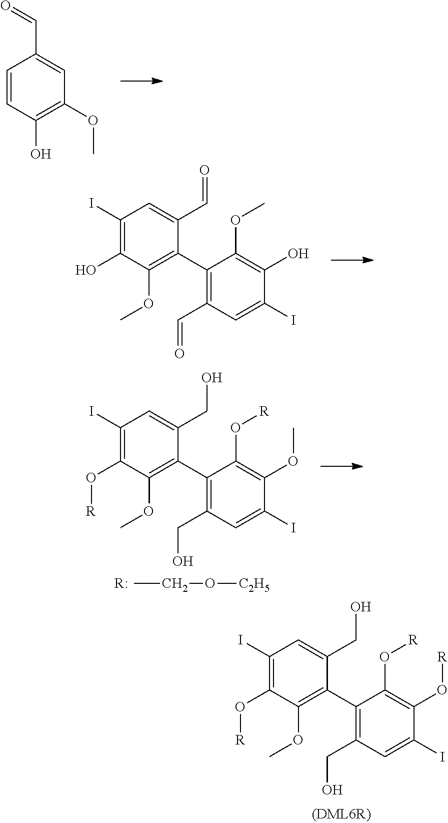

### Synthesis Example DML7

DML7 was synthesized in the same manner as in Synthesis Example DML1, except that 3,4-dihydroxybenzaldehyde was used as the raw material. Provided that, in the protective group introduction step, the amounts of diisopropylmethylamine and chloromethyl ethyl ether to 3,4-dihydroxybenzaldehyde as the raw material were set to 2 times.

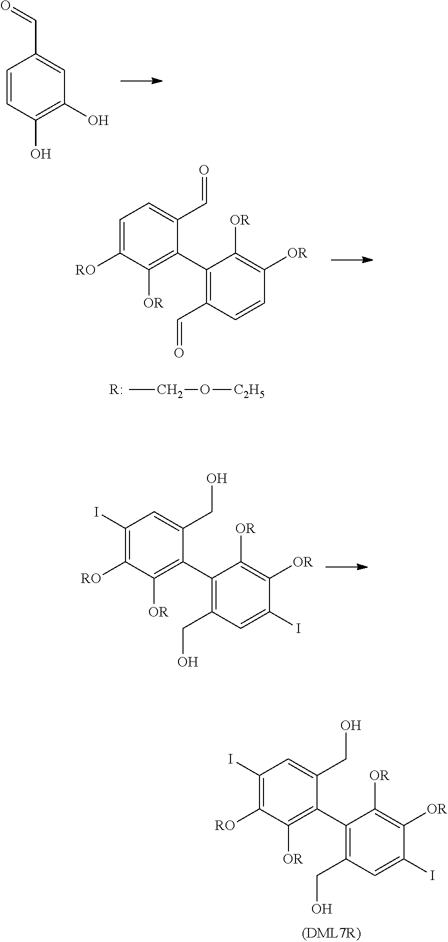

### Synthesis Example DML8

DML8R was synthesized in the same manner as in Synthesis Example DML1, except that ethylvanillin was used as the raw material.

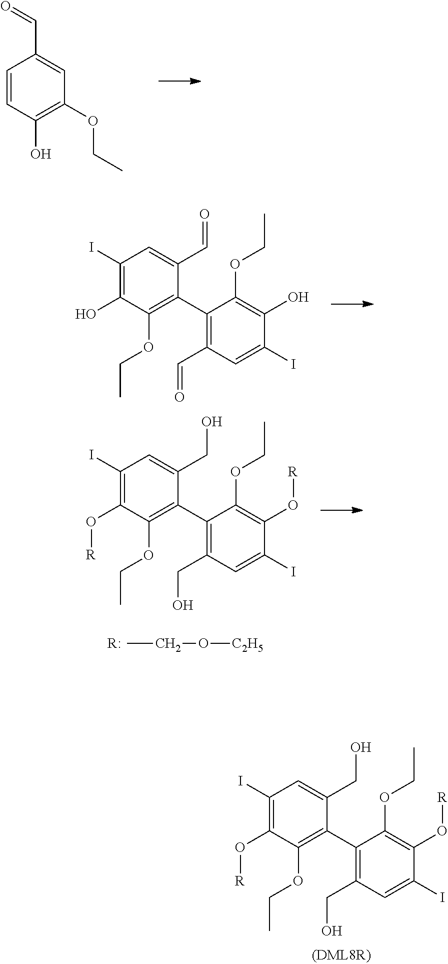

### Synthesis Example DML9

DML9R was synthesized in the same manner as in Synthesis Example DML1, except that 2-hydroxybenzaldehyde was used as the raw material.

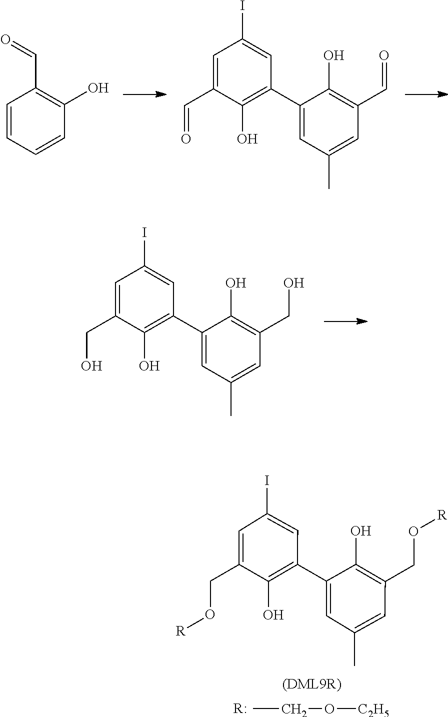

### [Example 2] Compound Having Naphthalene Ring as Core

The compound was produced according to the following scheme. The reaction was conducted under a nitrogen stream.

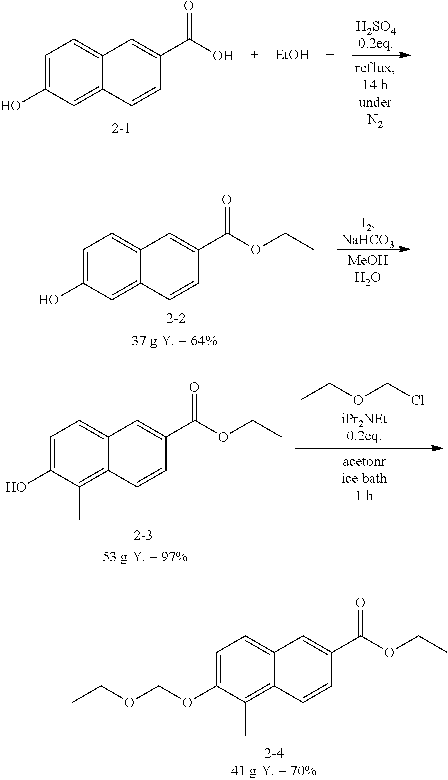

### Synthesis of Compound 2-2

A flask equipped with a stirrer and a condenser was charged with 50 g of the compound 2-1 (manufactured by FUJIFILM Wako Pure Chemical Corporation), 600 mL of ethanol, and 2.8 mL of 98% sulfuric acid (0.2 equivalents to the compound 2-1). The content was stirred under reflux for 14 hours and subjected to the esterification reaction. Then, the reaction mixture was neutralized with 10.6 g of sodium bicarbonate (reagent manufactured by FUJIFILM Wako Pure Chemical Corporation), and ethanol was distilled off. Then, ethyl acetate and water were added, and the organic phase was extracted, washed with sodium bicarbonate, then dehydrated with sodium sulfate to distill off the solvent, suspended and washed with acetone, and dried to obtain the compound 2-2. The yield was 64%.

### Synthesis of Compound 2-3

A flask equipped with a stirrer and a condenser was charged with 34 g of the compound 2-2, 48 g (1.2 equivalents to the compound 2-2) of iodine, 20 g of sodium bicarbonate, 160 mL of methanol, and 16 mL of water. The content was stirred at room temperature for 5 hours and subjected to the iodination reaction. Then, 50 mL of methanol was added, sodium hydrogen sulfite was added until the coloration of iodine was eliminated, and the mixture was filtered, washed with water, suspended and washed with methanol, filtered, and dried to obtain the compound 2-3. The yield was 97%.

### Synthesis of Compound 2-4

A flask equipped with a stirrer and a condenser was immersed in an ice bath, and the flask was charged with 50 g of the compound 2-3 and 150 mL of acetone, and stirred. At this time, the internal temperature was 4° C. Then, 20.8 g (1.1 equivalents to the compound 2-3) of diisopropylethylamine was added dropwise into the flask. After completion of the dropwise addition of diisopropylethylamine, 14.7 g of chloromethyl ethyl ether was added dropwise, and the reaction was carried out for 1 hour. After completion of the reaction, 300 g of ethyl acetate and 500 g of water were added, and the organic phase was extracted. The solvent of the extracted organic phase was distilled off and separated by column chromatography to obtain the compound 2-4. The yield was 70%.

### Synthesis of Na-1

The iodination step and the reduction step were carried out in the same manner as in Synthesis Example L1, except that 6,7,8-trimethoxynaphthalene-2-carbaldehyde was used instead of 4-hydroxybenzaldehyde, thereby obtaining the compound Na-1. The scheme is shown below.

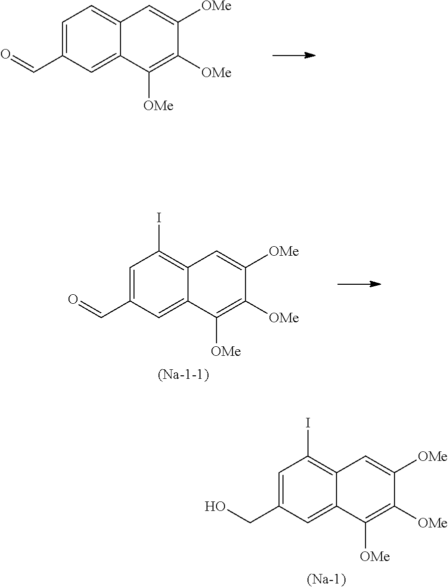 [SMILES: COc1cc2ccc(C=O)cc2c(OC)c1OC ; COc1cc2c(I)cc(C=O)cc2c(OC)c1OC ; COc1cc2c(I)cc(CO)cc2c(OC)c1OC ; "(Na-1)"]

### Synthesis of Na-2

The compound Na-2 was obtained in the same manner as in Synthesis Example L1, except that 2-hydroxy-3-naphthaldehyde was used instead of 4-hydroxybenzaldehyde. The scheme is shown below.

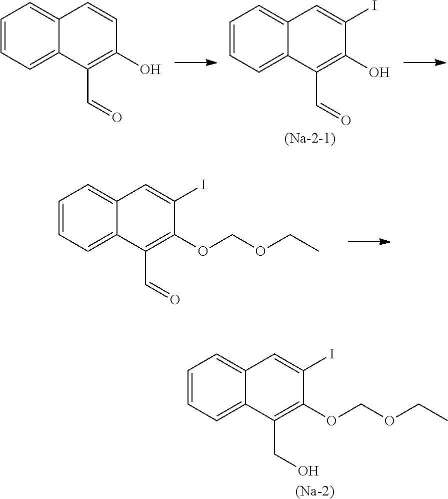

### Synthesis of Na-3

The compound Na-3 was obtained in the same manner as in Synthesis Example L1, except that 2-hydroxynaphthalene-6-carbaldehyde was used instead of 4-hydroxybenzaldehyde. The scheme is shown below.

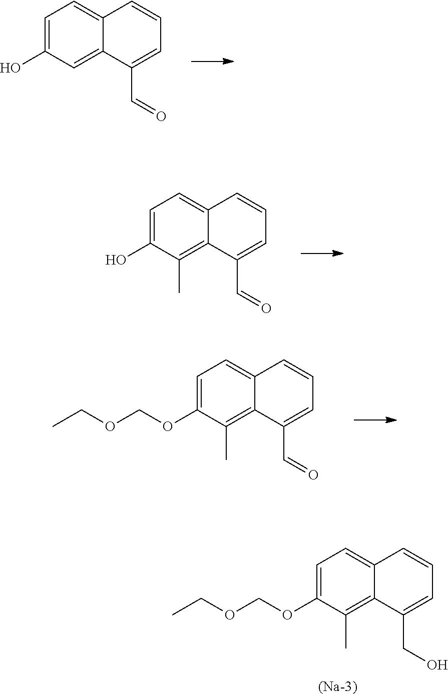

### Synthesis of Na-4

The compound Na-4-3 was obtained in the same manner as in Example 2, except that 3-hydroxy-2-naphthoic acid was used instead of the compound 2-1. The scheme is shown below.

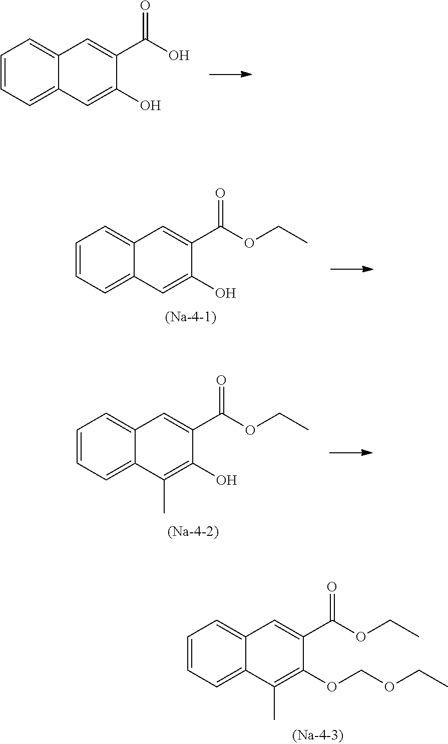 [SMILES: O=C(O)c1cc2ccccc2cc1O ; CCOC(=O)c1cc2ccccc2cc1O ; CCOC(=O)c1cc2ccccc2c(C)c1O ; CCOCOc1c(C(=O)OCC)cc2ccccc2c1C]

### Synthesis Example DMN2-3

The following reaction was conducted.

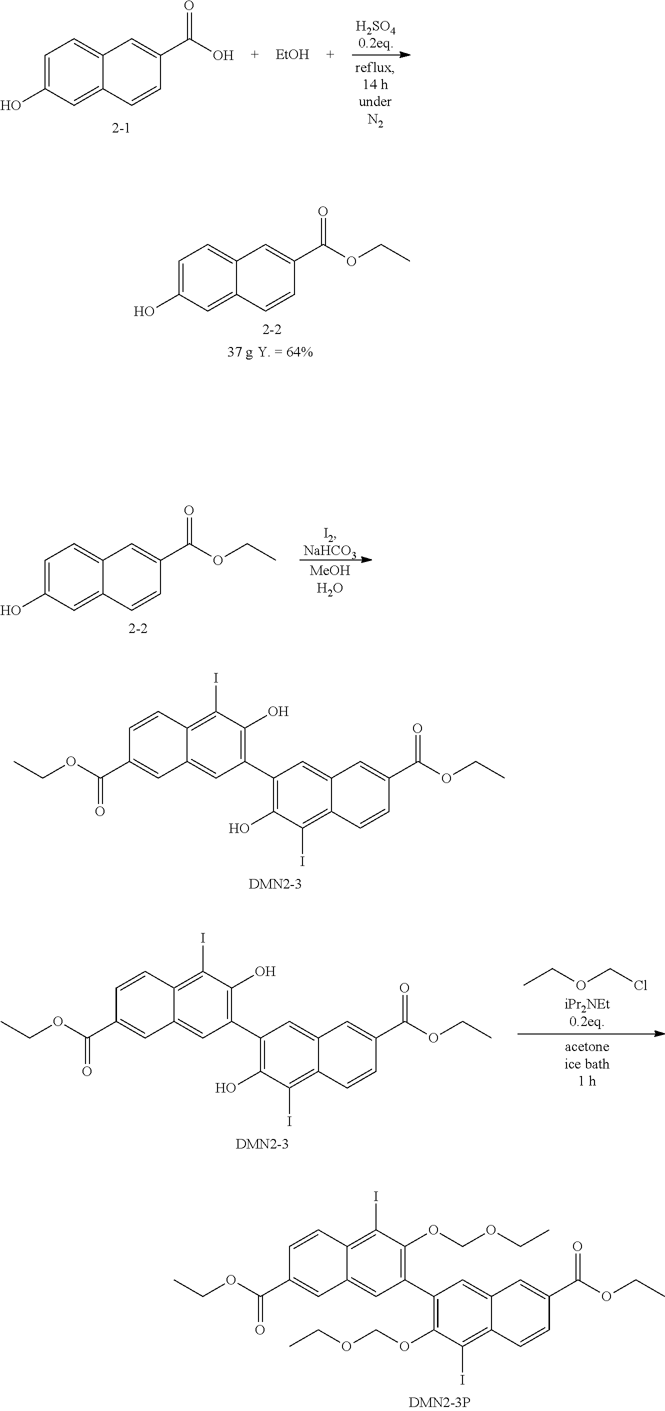

### Synthesis of Compound 2-2

The compound 2-2 was obtained in the same manner as in Example 2.

### Synthesis of Compound DMN2-3

A flask equipped with a stirrer and a condenser was charged with 34 g of the compound 2-2, 48 g (1.2 equivalents to the compound 2-2) of iodine, 10 g of sodium bicarbonate, 160 mL of methanol, and 16 mL of water. The content was stirred at 40° C. for 5 hours and subjected to the iodination reaction. Then, 50 mL of methanol was added, sodium hydrogen sulfite was added until the coloration of iodine was eliminated, and the mixture was filtered, washed with water, suspended and washed with methanol, filtered, and dried to obtain the compound DMN2-3.

### Synthesis of Compound DMN2-3P

A flask equipped with a stirrer and a condenser was immersed in an ice bath, and the flask was charged with 50 g of the compound DMN2-3 and 150 mL of acetone, and stirred. At this time, the internal temperature was 4° C. Then, 20.8 g (1.1 equivalents to DMN2-3) of diisopropylethylamine was added dropwise into the flask. After completion of the dropwise addition of diisopropylethylamine, 14.7 g of chloromethyl ethyl ether was added dropwise, and the reaction was carried out for 1 hour. After completion of the reaction, 300 g of ethyl acetate and 500 g of water were added, and the organic phase was extracted. The solvent of the extracted organic phase was distilled off and separated by column chromatography to obtain the compound DMN2-3P.

### Synthesis Example BPN2-3

The following reaction was carried out.

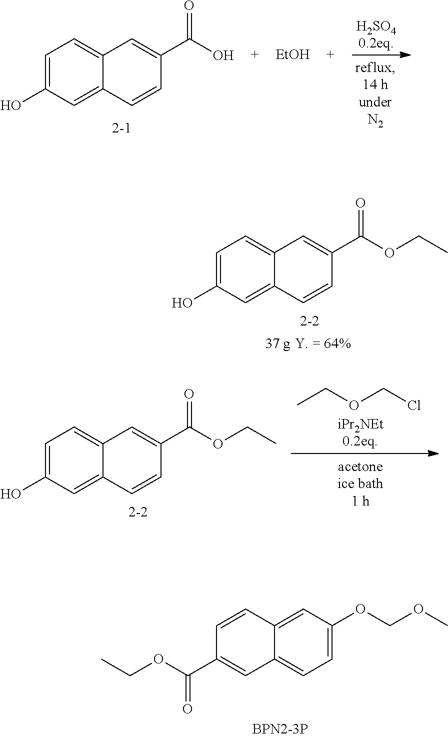

### Synthesis of Compound 2-2

The compound 2-2 was obtained in the same manner as in Example 2.

### Synthesis of Compound BPN2-3P

A flask equipped with a stirrer and a condenser was immersed in an ice bath, and the flask was charged with 15.8 g of the compound 2-2 and 150 mL of acetone, and stirred. At this time, the internal temperature was 4° C. Then, 20.8 g (1.1 equivalents to the compound 2-2) of diisopropylethylamine was added dropwise into the flask. After completion of the dropwise addition of diisopropylethylamine, 14.7 g of chloromethyl ethyl ether was added dropwise, and the reaction was carried out for 1 hour. After completion of the reaction, 300 g of ethyl acetate and 500 g of water were added, and the organic phase was extracted. The solvent of the extracted organic phase was distilled off and separated by column chromatography to obtain the compound BPN2-3P.

### Synthesis of DMNa-1-1

The compound DMNa-1-1 was synthesized in the same manner as in Synthesis Example DMN2-3, except that 6,7,8-trimethoxynaphthalene-2-carbaldehyde was used instead of the compound 2-2.

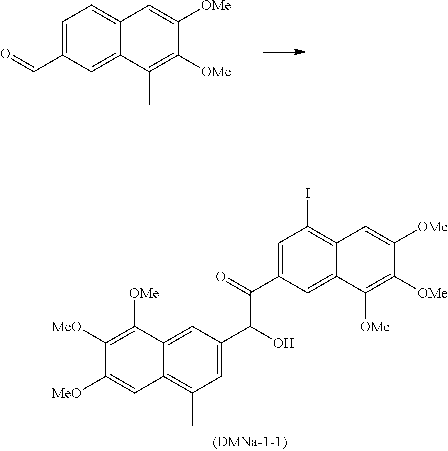 [SMILES: COc1cc2ccc(C=O)cc2c(C)c1OC ; COc1cc2c(C)cc(C(O)C(=O)c3cc(I)c4cc(OC)c(OC)c(OC)c4c3)cc2c(OC)c1OC]

### Synthesis Example DMNa2-1

The compound DMNa-2-1 was synthesized in the same manner as in Synthesis Example DMN2-3, except that 2-hydroxy-3-naphthaldehyde was used instead of the compound 2-2. Then, the compound DMNa2-1P was synthesized in the same manner as in the synthesis of the compound DMN2-3P, except that the compound DMNa-2-1 was used instead of the compound DMN2-3. Further, the compound DMNa-2-1R was synthesized in the same manner as in the synthesis of the compound 1-3, except that the compound DMNa2-1P was used instead of the compound 1-2.

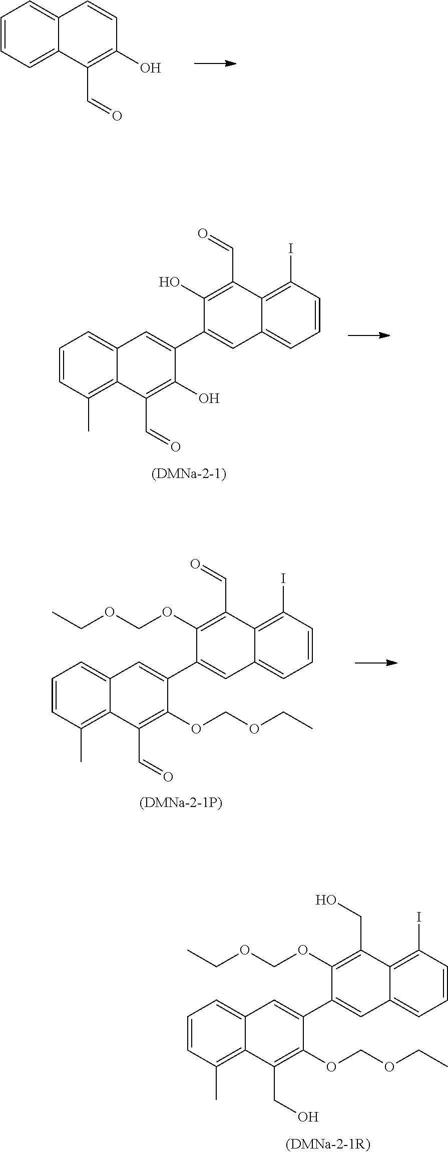 [SMILES: O=Cc1c(O)ccc2ccccc12 ; Cc1cccc2cc(-c3cc4cccc(I)c4c(C=O)c3O)c(O)c(C=O)c12 ; CCOCOc1c(-c2cc3cccc(I)c3c(C=O)c2OCOCC)cc2cccc(C)c2c1C=O ; CCOCOc1c(-c2cc3cccc(I)c3c(CO)c2OCOCC)cc2cccc(C)c2c1CO]

### Synthesis Example BPNa2-1

The compound BPNa2-1P was obtained in the same manner as in the synthesis of the compound DMN2-3P, except that 2-hydroxy-3-naphthaldehyde was used instead of the compound DMN2-3. Further, the compound BPNa-2-1R was obtained in the same manner as in the synthesis of the compound 1-3, except that the compound BPNa2-1P was used instead of the compound 1-2.

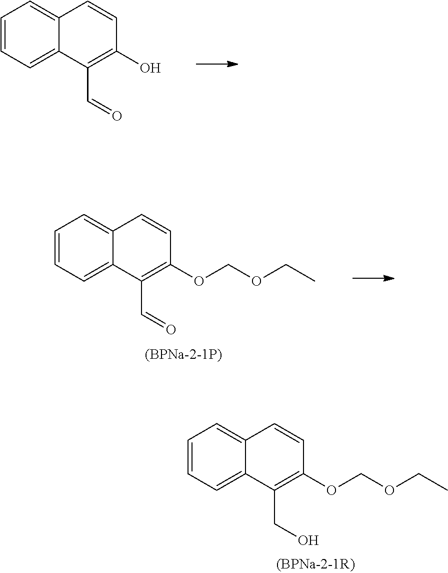 [SMILES: O=Cc1c(O)ccc2ccccc12 ; CCOCOc1ccc2ccccc2c1C=O ; CCOCOc1ccc2ccccc2c1CO]

### Synthesis of Compound DMNa-3-1

The compound DMNa-3-1 was obtained in the same manner as in Synthesis Example DMN2-3, except that 2-hydroxynaphthalene-6-carbaldehyde was used instead of the compound 2-2. Then, the compound DMNa3-1P was obtained in the same manner as in the synthesis of the compound DMN2-3P, except that the compound DMNa-3-1 was used instead of the compound DMN2-3. Further, the compound Na-3-1R was obtained in the same manner as in the synthesis of the compound 1-3, except that the compound DMNa3-1P was used instead of the compound 1-2.

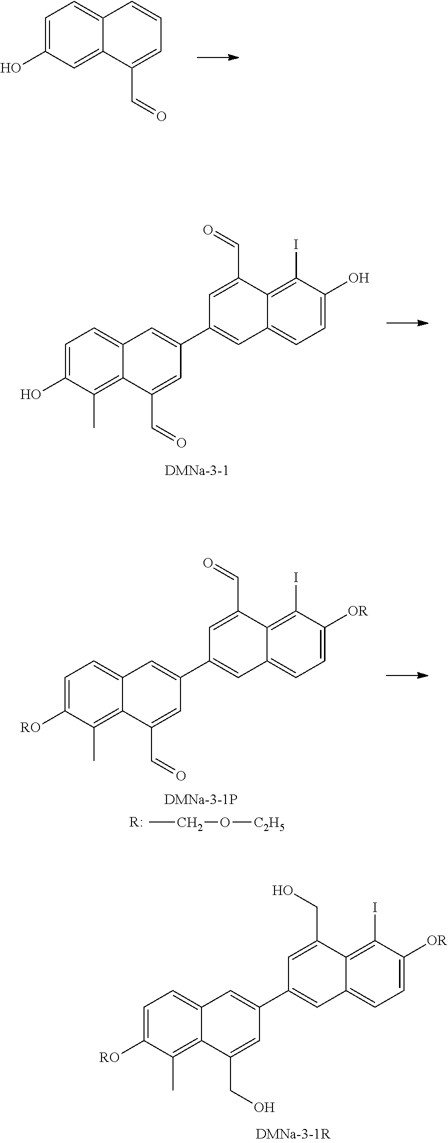

### Synthesis Example DMNa-2b-1R

The following reaction was carried out.

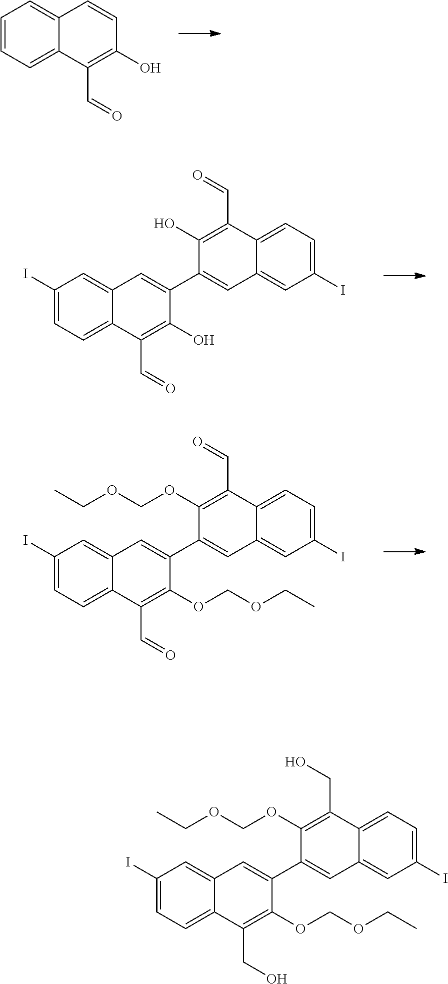 [SMILES: O=Cc1c(O)ccc2ccccc12 ; O=Cc1c(O)c(-c2cc3cc(I)ccc3c(C=O)c2O)cc2cc(I)ccc12 ; CCOCOc1c(-c2cc3cc(I)ccc3c(C=O)c2OCOCC)cc2cc(I)ccc2c1C=O ; CCOCOc1c(-c2cc3cc(I)ccc3c(CO)c2OCOCC)cc2cc(I)ccc2c1CO]

The compound DMNa-2b-1 was obtained in the same manner as in the iodination step DML1D, except that 2-hydroxy-3-naphthaldehyde was used instead of 4-hydroxybenzaldehyde. Then, the compound Na-2b-1P was obtained in the same manner as in the protective group introduction step DML1P, except that the compound DMNa-2b-1 was used instead of 5-iodovanillin. Further, the compound DMNa-2b-1R was obtained in the same manner as in the reduction step DML1R, except that the compound DMNa-2b-1P was used instead of DML1P.

### Synthesis of Synthesis Example DMNa-3-2R

The following reaction was carried out.

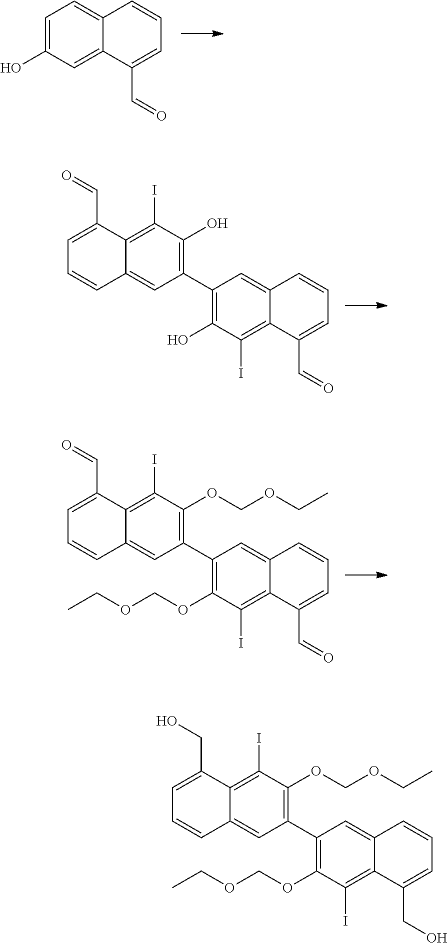 [SMILES: O=Cc1cccc2ccc(O)cc12 ; O=Cc1cccc2cc(-c3cc4cccc(C=O)c4c(I)c3O)c(O)c(I)c12 ; CCOCOc1c(-c2cc3cccc(C=O)c3c(I)c2OCOCC)cc2cccc(C=O)c2c1I ; CCOCOc1c(-c2cc3cccc(CO)c3c(I)c2OCOCC)cc2cccc(CO)c2c1I]

The iodination step, the protective group introduction step, and the reduction step were carried out in the same manner as in Synthesis Example DMNa-2-1, except that 2-hydroxynaphthalene-6-carbaldehyde was used as the raw material instead of 2-hydroxy-3-naphthaldehyde, thereby obtaining the compound DMNa-3-2R.

### Synthesis of DMNa-4-2P

The compound Na-4-1 was obtained in the same manner as in the synthesis of the compound 2-2, except that 3-hydroxy-2-naphthoic acid was used instead of the compound 2-1. Then, the compound DMNa-4-2 was obtained in the same manner as in the iodination step in Synthesis Example DMNa-2-1, except that the compound Na-4-1 was used instead of the compound 2-2. Further, the compound was subjected to the protective group introduction step in the same manner as in Synthesis Example DMNa-2-1, thereby obtaining the compound DMNa-4-2P.

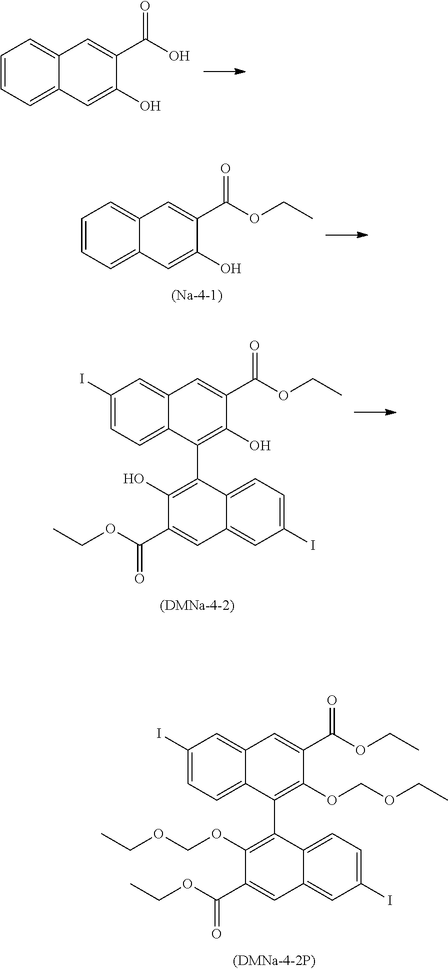

### [Example 3] Compound Having Adamantane Ring as Core

The compound was produced according to the following scheme.

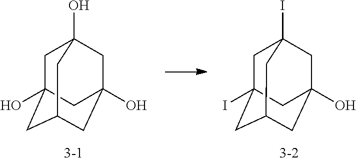 [SMILES: OC12CC3CC(O)(C1)CC(O)(C3)C2 ; OC12CC3CC(I)(C1)CC(I)(C3)C2]

A flask equipped with a stirrer and a condenser was immersed in an oil bath, and the flask was charged with 80 g of the compound 3-1 (0.43 mol, manufactured by Mitsubishi Gas Chemical Company, Inc.) and 2.5 L of toluene and stirred. Then, 400 g (1.72 mol) of a 55% aqueous hydrogen iodide solution was added into the flask. The internal temperature was set to 83 to 89° C., and the reaction was carried out for 32 hours. Further, 50 g of a 55% aqueous hydrogen iodide solution was added into the flask. The internal temperature was set to 83 to 89° C., and the reaction was carried out for 16 hours.

Another vessel was charged with 22.5 mL of a 10% aqueous sodium sulfite solution and 1720 mL of water, and the reaction solution was further slowly poured. 2 g of sodium sulfite and 1 L of ethyl acetate were further added, and the mixture was separated into an organic phase and an aqueous phase. Water was further added, and the mixture was separated to obtain an organic phase (oil phase). The organic phase was concentrated, 500 mL of toluene was added, and the mixture was left to stand in a freezer overnight.

The organic phase was filtered, and was washed with cooled toluene and hexane to obtain 145 g of a wet cake. The wet cake was dried under reduced pressure at 40° C. for 2.5 hours to obtain 138 g of a pale red crystal. Then, the crystal was mixed with 1.3 L of ethyl acetate and dissolved by heating the mixture to 70° C. The ethyl acetate solution was cooled to room temperature. To the solution, 650 mL of a 0.5% aqueous sodium sulfite solution was added, and the mixture was stirred and separated to take out the ethyl acetate phase.

To the ethyl acetate phase, 650 mL of water was added, and the mixture was stirred and separated. The ethyl acetate phase was taken out again, magnesium sulfate was added, and the mixture was stirred for 30 minutes and left to stand still in a refrigerator for two nights. Since a crystal was precipitated after leaving the mixture stand still, the mixture was heated to dissolve the crystal, and the mixture was cooled again to room temperature. The mixture after cooling was filtered to obtain magnesium sulfate and a filtrate. At this time, magnesium sulfate was washed with ethyl acetate. The filtrate thus obtained was concentrated and further dried under reduced pressure at 40° C. for 9 hours to obtain 128 g of a white crystal (compound 3-2).

### Synthesis of Ad-A-1

To 150 g of 1,3,5-adamantane triol, 1278 g of acetonitrile was added and stirred. 177 g of trimethylsilyl chloride was added at room temperature under nitrogen, and 244 g of sodium iodide (2 equivalents to 1,3,5-adamantane triol) was further added portionwise. The bath temperature was set to 85° C., and the mixture was reacted for 6 hours, and then allowed to cool overnight. After 1625 g of water was added, 79 g of a 10% aqueous sodium sulfite solution was added. The mixture was concentrated under reduced pressure using an evaporator until the content reached 2.2 kg, and 700 g of toluene was added. The mixture was stirred at room temperature for 14 hours and then filtered. The solid was washed twice with 150 ml of acetonitrile. The solid was dried under reduced pressure at 30° C. to obtain 132 g of a solid.

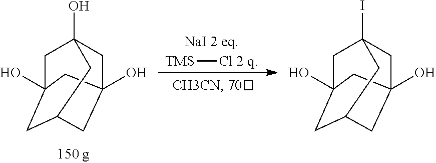

### Synthesis of Ad-A-2

To 24 g of 1-iodoadamantane-3,5-diol, 100 ml of dehydrated N-methylpyrrolidone (NMP) was added and ice cooled. 10 g of chloroacetyl chloride was added dropwise at 5° C. or less. The bath temperature was set to 60° C., and the mixture was reacted for 1 hour. The reaction solution was added to the ice to which 4% hydrochloric acid was added, while stirring. The mixture was extracted with 200 ml of ethyl acetate, and the extract was washed with 100 ml of 2% hydrochloric acid and 100 ml of water. After dehydration was carried out with brine and sodium sulfate, the solvent was distilled off by using an evaporator, and purification was carried out by silica gel column chromatography. 18.8 g of a solid was obtained.

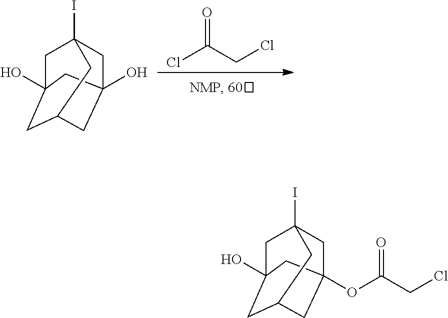 [SMILES: OC12CC3CC(O)(C1)CC(I)(C3)C2 ; O=C(Cl)CCl ; O=C(CCl)OC12CC3CC(O)(CC(I)(C3)C1)C2]

The compound Ad-2-2 was obtained in the same manner as in Example 3, except that the compound Ad-2-1 was used instead of the compound 3-1.

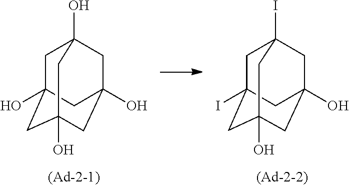 [SMILES: OC12CC3(O)CC(O)(C1)CC(O)(C2)C3 ; OC12CC3(O)CC(I)(C1)CC(I)(C2)C3]

10 g of Ad2-2 was dissolved in 30 ml of THF, which was ice cooled, and then, 7.7 g of succinyl chloride was added dropwise. After completion of the dropwise addition, the reaction was carried out at 60° C. for 2 hours. THF was distilled off from the reaction solution, toluene was added to the residue, and the precipitated solid was filtered to obtain 11 g of Ad-2-3.

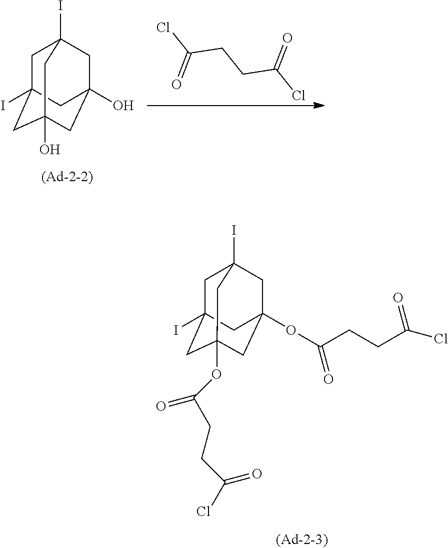 [SMILES: OC12CC3(O)CC(I)(C1)CC(I)(C2)C3 ; O=C(Cl)CCC(=O)Cl ; O=C(Cl)CCC(=O)OC12CC3(I)CC(I)(C1)CC(OC(=O)CCC(=O)Cl)(C3)C2]

### Synthesis of Ad-2-4

10 g of Ad-2-2 was dissolved in 50 ml of THF, which was ice cooled, and then, 7.7 g of succinyl chloride was added dropwise. After completion of the dropwise addition, the reaction was carried out at 60° C. for 2 hours. The reaction solution was cooled to room temperature, 40 ml of 15% sodium carbonate was then added dropwise, and the mixture was stirred for 1 hour. To the reaction solution, 7 ml of concentrated hydrochloric acid was added dropwise, and the precipitated solid was filtered to obtain 10 g of Ad-2-4.

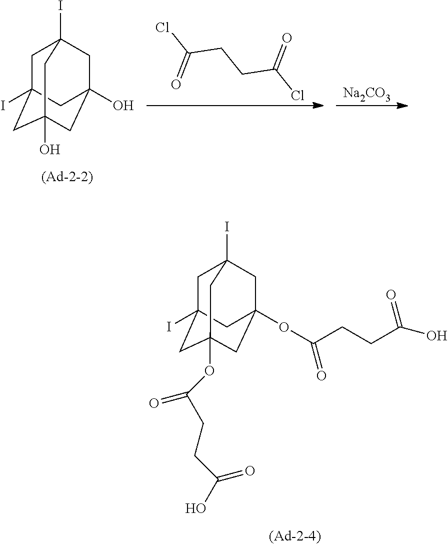

### [Synthesis Example DMA1] Compound Having Adamantane Ring as Core

The compound was produced according to the following scheme.

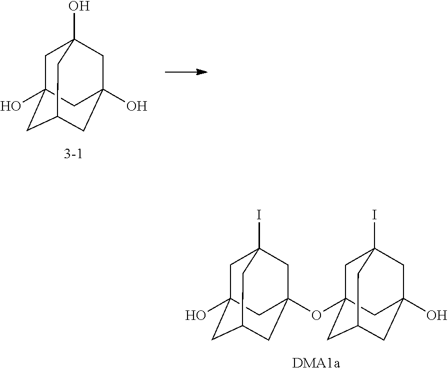 [SMILES: OC12CC3CC(O)(C1)CC(O)(C3)C2 ; OC12CC3CC(I)(C1)CC(OC14CC5CC(O)(CC(I)(C5)C1)C4)(C3)C2]

A flask equipped with a reflux tube and a Dienstark was immersed in an oil bath, and the flask was charged with 80 g of the compound 3-1 (0.43 mol, manufactured by Mitsubishi Gas Chemical Company, Inc.) and 2.5 L of o-xylene and stirred. Then, 400 g (1.72 mol of a 55% aqueous hydrogen iodide solution was added into the flask. The internal temperature was set to 125° C., and the reaction was carried out for 3 hours. Thereafter, the reaction solution was stirred in a water bath at 25° C. for 1 hour.

To the reaction vessel, 2.5 L of ion-exchange water was additionally added, and the mixture was separated to an organic phase and an aqueous phase. The organic phase was recovered, and then subjected to washing treatment with 1 L of a 5% sodium bicarbonate solution and further, 1 L of ion-exchange water, three times. The organic phase was recovered and then concentrated to obtain 35 g of a white solid (DMA1a).

To a 3 L flask connected with a reflux tube and a Dienstark, 35 g of DMA1a (0.06 mol) and 182.5 mL of toluene subjected to dehydration treatment were put under nitrogen flow, and the mixture was stirred with a stirring blade for dissolution. Then, 12 g of methanesulfonic acid was added over 30 minutes while stirring for dissolution. Further, 7.4 g (1.2 equivalents to the substrate) of succinic anhydride was added to the stirred reaction solution over 60 minutes, the internal temperature was then set to 100° C., and the mixture was stirred for 2 hours. Thereafter, the reaction solution was cooled to room temperature, 360 mL of pure water was added, the mixture was subjected to separation treatment, and then, the toluene phase was recovered. Further, the toluene phase was subjected to washing treatment with 360 mL of a 0.5% aqueous sodium bicarbonate solution, and then washed three times with 350 mL of ion-exchange water, and the recovered toluene phase was concentrated under reduced pressure to obtain 20.6 g of a white solid as the protected compound DMA1aP which is the target substance. The yield was 50%.

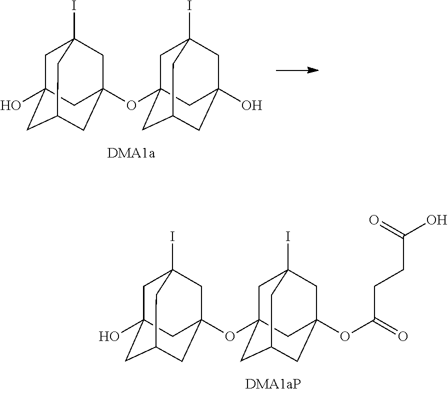 [SMILES: OC12CC3CC(I)(C1)CC(OC14CC5CC(O)(CC(I)(C5)C1)C4)(C3)C2 ; O=C(O)CCC(=O)OC12CC3CC(I)(C1)CC(OC14CC5CC(O)(CC(I)(C5)C1)C4)(C3)C2]

### [Synthesis Example DMA2] Compound Having Adamantane Ring as Core

The compound was produced according to the following scheme.

### Synthesis of DMA2

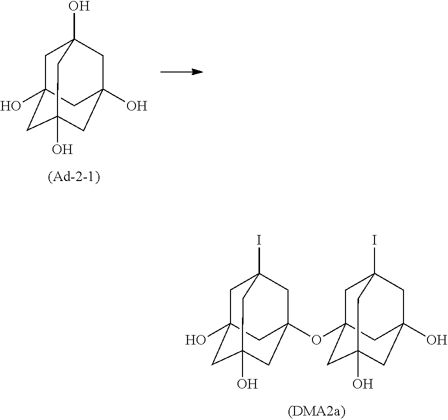 [SMILES: OC12CC3(O)CC(O)(C1)CC(O)(C2)C3 ; OC12CC3(O)CC(I)(C1)CC(OC14CC5(O)CC(O)(CC(I)(C5)C1)C4)(C2)C3]

A flask equipped with a reflux tube and a Dienstark was immersed in an oil bath, and the flask was charged with 87.9 g of the compound Ad-2-1 (0.43 mol) and 2.5 L of toluene and stirred. Then, 400 g (1.72 mol) of a 55% aqueous hydrogen iodide solution was added into the flask. The internal temperature was set to 100° C., and the reaction was carried out for 3 hours. Thereafter, the reaction solution was stirred in a water bath at 25° C. for 1 hour.

To the reaction vessel, 2.5 L of ion-exchange water was additionally added, and the mixture was separated to an organic phase and an aqueous phase. The organic phase was recovered, and then subjected to washing treatment with 1 L of a 5% sodium bicarbonate solution and further, 1 L of ion-exchange water, three times. The organic phase was recovered and then concentrated to obtain 37 g of a white solid (DMA2a).

### Synthesis of DMA2P

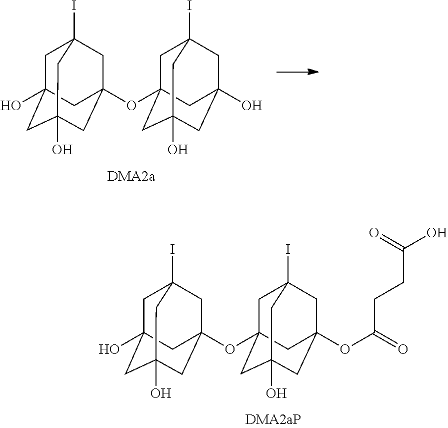 [SMILES: OC12CC3(O)CC(I)(C1)CC(OC14CC5(O)CC(O)(CC(I)(C5)C1)C4)(C2)C3 ; O=C(O)CCC(=O)OC12CC3(O)CC(I)(C1)CC(OC14CC5(O)CC(O)(CC(I)(C5)C1)C4)(C3)C2]

To a 3 L flask connected with a reflux tube and a Dienstark, 37 g (0.06 mol) of (DMA1a) and 182.5 mL of toluene subjected to dehydration treatment were put under nitrogen flow, and the mixture was stirred with a stirring blade for dissolution. Then, 12 g of methanesulfonic acid was added over 30 minutes while stirring for dissolution. Further, 7.4 g (1.2 equivalents to the substrate) of succinic anhydride was added to the stirred reaction solution over 60 minutes, the internal temperature was then set to 100° C., and the mixture was stirred for 2 hours. Thereafter, the reaction solution was cooled to room temperature, 360 mL of pure water was added, the mixture was subjected to separation treatment, and then, the toluene phase was recovered. Further, the toluene phase was subjected to washing treatment with 360 mL of a 0.5% aqueous sodium bicarbonate solution, and then washed three times with 350 mL of ion-exchange water, and the recovered toluene phase was concentrated under reduced pressure to obtain 23.8 g of a white solid as the protected compound DMA2aP which is the target substance. The yield was 55%.

### Synthesis of DMA1P2

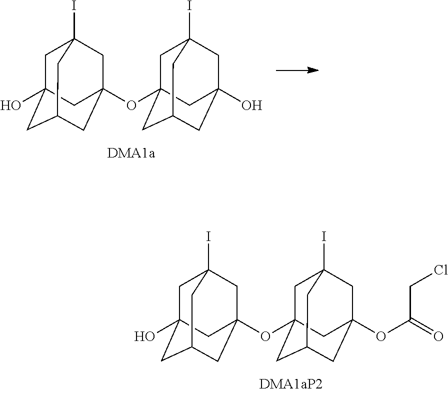 [SMILES: OC12CC3CC(I)(C1)CC(OC14CC5CC(O)(CC(I)(C5)C1)C4)(C3)C2 ; O=C(CCl)OC12CC3CC(I)(C1)CC(OC14CC5CC(O)(CC(I)(C5)C1)C4)(C3)C2]

To 47.1 g of DMA1a, 140 ml of the hydrated NMP was added, and the mixture was ice cooled. 10 g of chloroacetyl chloride was added dropwise at 5° C. or less. The bath temperature was set to 60° C., and the mixture was reacted for 1 hour. The reaction solution was added to the ice to which 4% hydrochloric acid was added, while stirring. The mixture was extracted with 200 ml of ethyl acetate, and the extract was washed with 100 ml of 2% hydrochloric acid and 100 ml of water. After dehydration was carried out with brine and sodium sulfate, the solvent was distilled off by an evaporator, and purification was carried out by silica gel column chromatography. 29.6 g of a solid was obtained as DMA1aP2 which is the target substance in a yield of 79.7%.

### Synthesis of DMA2P2

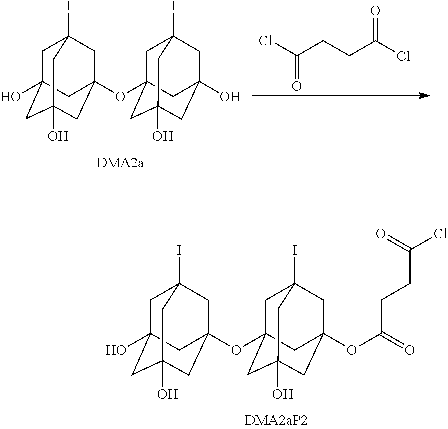 [SMILES: OC12CC3(O)CC(I)(C1)CC(OC14CC5(O)CC(O)(CC(I)(C5)C1)C4)(C2)C3 ; O=C(Cl)CCC(=O)Cl ; O=C(Cl)CCC(=O)OC12CC3(O)CC(I)(C1)CC(OC14CC5(O)CC(O)(CC(I)(C5)C1)C4)(C3)C2]

140 ml of THF was added to 49.1 g of DMA2a, and the mixture was ice cooled. 14 g of succinyl chloride was added dropwise at 5° C. or less. The bath temperature was set to 60° C., and the mixture was reacted for 2 hours. After completion of the dropwise addition, the reaction was carried out at 60° C. for 2 hours. THF was distilled off from the reaction solution, toluene was added to the residue, and 46.9 g of the precipitated solid was recovered by filtration to obtain DMA2aP2 which is the target substance in a yield of 79.8%.

### [Examples 4 to 7] Composition for Lithography

### (Base Material A)

0.5 g of 4-hydroxystyrene, 4.0 g of 2-methyl-2-adamantyl methacrylate, 0.9 g of γ-butyrolactone methacrylate, and 1.5 g of hydroxyadamantyl methacrylate were dissolved in 45 mL of tetrahydrofuran, and 0.20 g of azobisisobutyronitrile was added thereto. After the mixture was refluxed for 12 hours, the reaction solution was added dropwise to 2 L of n-heptane. The polymer precipitated was filtered off and dried under reduced pressure to obtain a white powdery polymer MAR represented by the following formula (MAR). This polymer had a weight average molecular weight (Mw) of 11,500 and a dispersity (Mw/Mn) of 1.90. As a result of the measurement of ¹³C-NMR, the composition ratio (molar ratio) in the following formula (MA1) was a:b:c:d=60:10:15:15. Although the following formula (MAR) is illustrated in a simplified form to show the ratio of respective constitutional units, the order of arrangement of respective constitutional units is random and this polymer is not a block copolymer in which respective constitutional units form an independent block. The molar ratio was determined based on the integral ratio of each of the carbon of the main chain directly bonded to a benzene ring for the unit having a benzene ring and the carbonyl carbon of an ester bond for a methacrylate unit (2-methyl-2-adamantyl methacrylate, γ-butyrolactone methacrylate, and hydroxyadamantyl methacrylate).

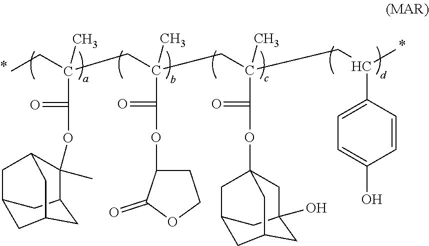

### (Composition)

The compositions shown in Table 1 were prepared by using the compound 1-3 synthesized in Example 1, the compound 2-3 and the compound 2-4 synthesized in Example 2, or the compound 3-2 synthesized in Example 3 as the compound B. The following acid generating agent, acid diffusion controlling agent, and organic solvent were used.

Acid generating agent: triphenylsulfonium nonafluorobutanesulfonate (TPS-109) manufactured by Midori Kagaku Co., Ltd.

Acid diffusion controlling agent: tri-n-octylamine (TOA) manufactured by Kanto Chemical Co., Inc.

Organic solvent: propylene glycol monomethyl ether acetate (PGMEA) manufactured by Kanto Chemical Co., Inc.

TABLE 1

| | | | Acid | | |
|---|---|---|---|---|---|
| | | | generating | Acid diffusion | Organic |
| | | | agent | controlling | solvent |
| | Base material A | Compound B | (parts by | agent | (parts by |
| | (parts by mass) | (parts by mass) | mass) | (parts by mass) | mass) |
| Example 4 | MAR | Compound 1-3 | TPS-109 | TOA | PGMEA |
| | (4.8) | (0.2) | (0.5) | (0.05) | (100) |
| Example 5 | MAR | Compound 2-3 | TPS-109 | TOA | PGMEA |
| | (4.8) | (0.2) | (0.5) | (0.05) | (100) |
| Example 6 | MAR | Compound 2-4 | TPS-109 | TOA | PGMEA |
| | (4.8) | (0.2) | (0.5) | (0.05) | (100) |
| Example 7 | MAR | Compound 3-2 | TPS-109 | TOA | PGMEA |
| | (4.8) | (0.2) | (0.5) | (0.05) | (100) |
| Comparative | MAR | None | TPS-109 | TOA | PGMEA |
| Example 1 | (5.0) | | (0.5) | (0.05) | (100) |

### [Evaluation Method]

### (Pattern Evaluation of EB Resist Pattern (Pattern Formation))

After each resist composition was prepared according to the composition in Table 1, a clean silicon wafer was spin coated with the resist composition, and then prebaked (PB) before exposure on a hot plate of 110° C. to form a resist film with a thickness of 50 nm. The obtained resist film was irradiated with electron beams of 1:1 line and space setting with a 50 nm interval using an EB lithography system (ELS-7500 manufactured by ELIONIX INC.). After irradiation, the resist film was heated at 110° C. for 90 seconds, and immersed in a 2.38 mass % TMAH alkaline developing solution for 60 seconds for development. Thereafter, the resist film was washed with ultrapure water for 30 seconds, and dried to form a resist pattern.

The cross-sectional shape of the obtained resist patterns of 50 nmL/S (1:1) was observed using an electron microscope manufactured by Hitachi Ltd. (S-4800). With respect to the resist pattern shape after development, those in which the pattern width at a position 10% of the pattern height from the surface of the silicon wafer relative to the half-width of the pattern cross section is less than +10% of the half-width were evaluated to be “A”, and those in which the pattern width is +10% or more of the half-width were evaluated to be “C”. Furthermore, the smallest electron beam energy quantity capable of lithographing a shape having no pattern collapse was set as “electron beam lithographic sensitivity”, and those in which the electron beam lithographic sensitivity is equivalent to or superior to Comparative Example 1 were evaluated to be “A”, and those in which the electron beam lithographic sensitivity is inferior to Comparative Example 1 were evaluated to be “C”.

TABLE 2

| | Resist pattern | Sensitivity |
|---|---|---|
| Example 4 | A | A |
| Example 5 | A | A |
| Example 6 | A | A |
| Example 7 | A | A |
| Comparative | A | A |
| Example 1 | | |

### [Examples 8 to 11] EUV Exposure Sensitivity, Etching Defect

### (EUV Exposure Sensitivity)

A silicon wafer was spin coated with each composition prepared in Examples 4 to 7, which was then baked at 110° C. for 60 seconds to form a photoresist layer having a film thickness of 100 nm. For the composition of Comparative Example 3, the compound 3-1 was used instead of the compound 1-3 of Example 4. Then, using an extreme ultraviolet (EUV) exposure apparatus “EUVES-7000” (product name, manufactured by Litho Tech Japan Corporation), the photoresist layer was subjected to shot exposure without masking by incrementing the exposure amount by 1 mJ/cm² from 1 mJ/cm² to 80 mJ/cm², baked (PEB) at 110° C. for 90 seconds, and developed with a 2.38% by mass aqueous tetramethylammonium hydroxide (TMAH) solution for 60 seconds to obtain a wafer on which 80 shots of shot exposure was carried out. For each obtained shot exposure area, the film thickness was measured by using an optical interference film thickness meter “VM3200” (product name, manufactured by SCREEN Semiconductor Solutions Co., Ltd.), profile data about the film thickness relative to the exposure amount was obtained, and the exposure amount at which the slope of film thickness variation relative to an exposure amount was the largest was calculated as a sensitivity value (mJ/cm²), which was used as an index of the EUV sensitivity of the resist.

### (Evaluation of Etching Defect)

The composition used in the measurement of EUV exposure sensitivity was applied on a 8 inch silicon wafer, on the outermost layer of which an oxide film having a film thickness of 100 nm was formed, and this was baked at 110° C. for 60 seconds to form a photoresist layer having a film thickness of 100 nm. Then, using an extreme ultraviolet (EUV) exposure apparatus “EUVES-7000” (product name, manufactured by Litho Tech Japan Corporation), the photoresist layer was subjected to shot exposure with an exposure amount that is 10% less than the EUV sensitivity value obtained in the aforementioned EUV sensitivity evaluation on the entire wafer surface, further baked (PEB) at 110° C. for 90 seconds, and developed with a 2.38% by mass aqueous tetramethylammonium hydroxide (TMAH) solution for 60 seconds to obtain a wafer on the entire surface of which 80 shots of shot exposure was carried out.

The prepared exposed wafer was subjected to an etching treatment with an etching apparatus “Telius SCCM” (product name, manufactured by Tokyo Electron Ltd.), using CF₄/Ar gas until the oxide film was etched by 50 nm. Evaluation of defects were carried out for the wafer prepared by etching with a defect inspection apparatus “Surfscan SP5” (product name, manufactured by KLA), and the number of cone defects with a size of 19 nm or more was determined as an index of the etching defect.

### (Evaluation Criteria)

A: Number of cone defects ≤10
B: 10<number of cone defects ≤80
C: 80<number of cone defects ≤400
D: 400<number of cone defects
   (Change of Evaluation of Etching Defect with Time)

The composition used in the evaluation of the etching defect was left to stand at room temperature for 7 days, and the evaluation of the etching defect was carried out again. The case where the change in the EUV sensitivity before and after standing is less than 6% was evaluated to be “G”, and the case where the change is 6% or more was evaluated to be “N”.

TABLE 3

| | | EUV sensitivity | Etching | Change of etching |
|---|---|---|---|---|
| | Composition used | (mJ/cm²) | defect | defect with time |
| Example 8 | Composition used in | 22 | A | G |
| | Example 4 | | | |
| Example 9 | Composition used in | 27 | B | G |
| | Example 5 | | | |
| Example 10 | Composition used in | 28 | A | G |
| | Example 6 | | | |
| Example 11 | Composition used in | 22 | B | G |
| | Example 7 | | | |
| Comparative | Composition used in | 49 | C | G |
| Example 2 | Comparative Example 1 | | | |
| Comparative | Composition in which | 50 | C | G |
| Example 3 | compound 3-1 was used | | | |
| | instead of compound 1-3 | | | |
| | of Example 4 | | | |

### [Example 12] Purified Product with Acid of Compound 3-2

(Treatment 1: Purification with Acid)

A four necked flask (capacity: 1000 mL, with a detachable bottom) was charged with 150 g of a solution (10% by mass) formed by dissolving the compound 3-2 in PGMEA, and was heated to 80° C. while stirring. Then, 37.5 g of an aqueous oxalic acid solution (pH 1.3) was added thereto, and the resultant mixture was stirred for 5 minutes and then left to stand still for 30 minutes. This separated the mixture into an oil phase and an aqueous phase, and the aqueous phase was thus removed. After repeating this operation once, 37.5 g of ultrapure water was charged to the obtained oil phase, and after stirring for 5 minutes, the mixture was left to stand for 30 minutes and the aqueous phase was removed. After repeating this operation three times, the residual water and PGMEA were concentrated and removed by heating to 80° C. and reducing the pressure in the flask to 200 hPa or less. By diluting with PGMEA of EL grade (a reagent manufactured by Kanto Chemical Co., Inc.) such that the concentration of the compound 3-2 in PGMEA solution was adjusted to 10% by mass, a PGMEA solution of the compound 3-2 with a reduced metal content was obtained.

(Treatment 2: Treatment without Acid)

A PGMEA solution of the compound 3-2 whose concentration was adjusted to 10% by mass was obtained in the same manner as in Example 12, except that ultrapure water was used instead of the aqueous oxalic acid solution.

For the 10% by mass PGMEA solution of the compound 3-2 without treatment, the 10% by mass PGMEA solution of the compound 3-2 subjected to the treatment 1, and the 10% by mass PGMEA solution of the compound 3-2 subjected to the treatment 2, each metal content was measured by ICP-MS. The measurement results are shown in Table 4.

TABLE 4

| | Metal content (ppb) | | |
|---|---|---|---|
| | Na | K | Fe |
| Before treatment | >99 | >99 | >99 |
| Compound 3-2 | | | |
| Example 12 | 1.5 | <0.2 | 1.2 |
| Treatment 1 | | | |
| Example 12 | 3.0 | 1.3 | >99 |
| Treatment 2 | | | |

### (EUV Exposure Sensitivity, Etching Defect)

The EUV exposure sensitivity and the etching defect were measured by using the compound 3-2 after purification in the same manner as in Example 8. The measurement results are shown in Table 5.

TABLE 5

| | EUV sensitivity | |
|---|---|---|
| | (mJ/cm²) | Etching defect |
| Example 12 | 23 | A |
| Treatment 1 | | |
| Example 12 | 23 | B |
| Treatment 2 | | |
| (Reference) | 22 | B |
| Example 11 | | |

### [Example 13] Synthesis of Aldehyde Having Benzene Ring as Core

The compound was produced according to the following scheme. The reaction was conducted under a nitrogen stream.

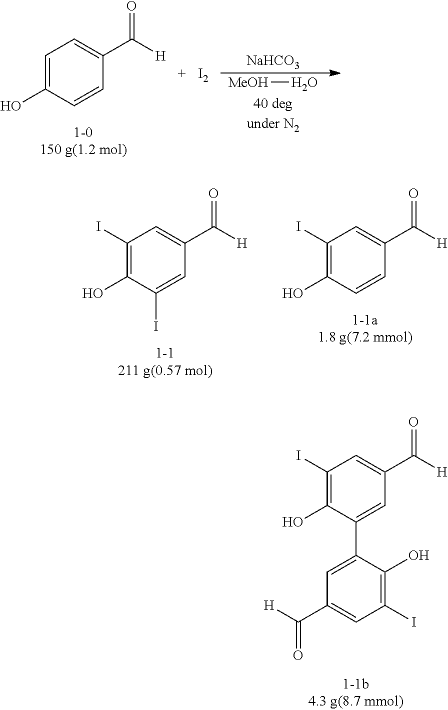

### Synthesis of Compound 1-1, Compound 1-1a, and Compound 1-1b

In a 3 L three necked flask to which a stirrer and a nitrogen flow were attached, 150 g (1.2 mol) of 4-hydroxybenzaldehyde and 1 L of methanol (Kanto Chemical Co., Inc.) were weighed. The flask was immersed in a water bath, the temperature of the water bath was set to 40° C., the flask was heated under a nitrogen stream while stirring, and 200 mL of water was added. At the time when the internal temperature reached 34° C., 309.4 g (3.7 mol) of sodium bicarbonate (NaHCO₃) was added in one portion.

At the time when the internal temperature reached 38° C., 654.5 g (2.6 mol) of iodine (I₂) was added in portions while paying attention to bubbling, and the mixture was stirred at 40° C. for 3 hours. Thereafter, the flask was cooled with water, and an aqueous sodium sulfite solution (Na₂SO₃) was added dropwise until the color of the reaction solution was changed to yellowish-white.

In a vessel to which a stirrer was attached, 3 L of water was put, the reaction solution was poured, and the mixture was stirred for 15 minutes. The precipitate was filtered and washed with 500 mL of water. The filtrate was put in a vessel to which a stirrer was attached, 1 L of water was added, and the mixture was stirred for 15 minutes. The precipitate was filtered and washed with 300 mL of water.

The filtrate was put in a vessel to which a stirrer was attached, 500 mL of methanol was added, and the mixture was stirred for 15 minutes. The precipitate was filtered and washed with 150 mL of methanol. Using column chromatography (spherical silica 60N manufactured by Kanto Chemical Co., Inc.), the precipitate was separated by applying gradient so that the ratio of ethyl acetate:hexane as the developing solvent was 1:9 to 9:1, thereby obtaining the compound 1-1, the compound 1-1a, and the compound 1-1b in a relative value ratio of each amount of about 1:0.9:0.5.

### Synthesis of Compound 1-3a and Compound 1-3b

The compound 1-3a was obtained from the compound 1-1a through the compound 1-2a in the same manner as in Example 1. The compound 1-3b was obtained from the compound 1-1b through the compound 1-2b.

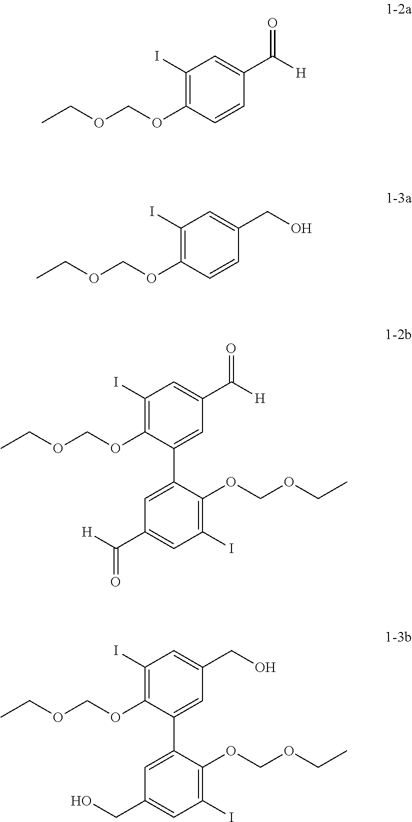

### Synthesis of mixture of compounds 1-2, 1-2a, and 1-2b, and mixture of compounds 1-3, 1-3a, and 1-3b

The mixture of the compounds 1-2, 1-2a, and 1-2b was obtained from the mixture of the compounds 1-1, 1-1a, and 1-1b in the same manner as in Example 1. Furthermore, the mixture of the compounds 1-3, 1-3a, and 1-3b was obtained from the mixture of the compounds 1-2, 1-2a, and 1-2b.

### [Examples 14 to 18] Evaluation of EUV Exposure Sensitivity and Etching Defect

Each composition was prepared in the same manner as in Example 4, by using the following compounds as the compound B, and the EUV exposure sensitivity and the etching defect were evaluated in the same manner as in Example 8. Provided that, the etching defect was evaluated according to the following criteria.

### (Evaluation Criteria)

S: Number of cone defects ≤6
A′: 6<number of cone defects ≤10
B: 10<number of cone defects ≤80
C: 80<number of cone defects ≤400
D: 400<number of cone defects

TABLE 5

| | | EUV sensitivity | |
|---|---|---|---|
| | Compound B | (mJ/cm²) | Etching defect |
| Example 14 | Compound 1-3 | 22 | A′ |
| Example 15 | Compound 1-3/1-3a = | 22 | S |
| | 99.5/0.5 | | |
| Example 16 | Compound 1-3/1-3b = | 22 | S |
| | 99.5/0.5 | | |
| Example 17 | Compound 1-3/1-3a/1- | 22 | S |
| | 3b = 99.5/0.25/0.25 | | |
| Example 18 | Compound 1-3/1-3a/1- | 23 | S |
| | 3b = 99/0.5/0.5 | | |

The above results show that the compound of the present embodiment has industrial applicability such that the composition for lithography having high sensitivity in EUV exposure and less defects can be provided while maintaining a good pattern shape.

### Example B

Evaluations were carried out in the same manner as in Examples 4 and 8, except that the compound B described in Table 6 was used instead of the compound 1-3 described in Example 14 in Table 5, and furthermore, as the evaluation conditions, the baking temperature after exposure was set to 100° C. for 120 seconds. As a result, as described in Table 7, good evaluation results as in Examples 4 and 8 were confirmed with respect to both the resist pattern and the sensitivity.

TABLE 6
Examples B1 to B35

| Examples | Compound B1 |
|---|---|
| Example B1 | 1-3 |
| Example B5 | 2-4 |
| Example B7 | Ad-2-2 |
| Example B8 | 3-2 |
| Example B9 | 6 |
| Example B11 | Na-2 |
| Example B12 | 2 |
| Example B13 | 3 |
| Example B14 | 4 |
| Example B15 | 5 |
| Example B16 | 1-7 |
| Example B18 | 1-8 |
| Example B19 | 1-5 |
| Example B20 | 1-6 |
| Example B21 | 1-4 |
| Example B25 | 2-3 |
| Example B26 | Na-1 |
| Example B27 | Na-2 |
| Example B28 | Na-3 |
| Example B29 | Na-4-2 |
| Example B30 | Na-4-3 |
| Example B31 | Ad-A-1 |
| Example B32 | Ad-A-2 |
| Example B33 | Ad-2-2 |
| Example B34 | Ad-2-3 |
| Example B35 | Ad-2-4 |

TABLE 7
Examples B1 to B35

| | | | | | Change of |
|---|---|---|---|---|---|
| | | | EUV | | etching |
| | Resist | | sensitivity | Etching | defect |
| Examples | pattern | Sensitivity | (mJ/cm²) | defect | with time |
| Example B1 | A | A | 22 | A | G |
| Example B5 | A | A | 28 | A | G |
| Example B7 | A | A | 26 | A | G |
| Example B8 | A | A | 22 | A | G |
| Example B9 | A | A | 24 | A | G |
| Example B11 | A | A | 26 | A | G |
| Example B12 | A | A | 27 | A | G |
| Example B13 | A | A | 25 | A | G |
| Example B14 | A | A | 24 | A | G |
| Example B15 | A | A | 27 | A | G |
| Example B16 | A | A | 26 | A | G |
| Example B18 | A | A | 25 | A | G |
| Example B19 | A | A | 26 | A | G |
| Example B20 | A | A | 25 | A | G |
| Example B21 | A | A | 24 | A | G |
| Example B25 | A | A | 27 | A | G |
| Example B26 | A | A | 24 | A | G |
| Example B27 | A | A | 27 | A | G |
| Example B28 | A | A | 26 | A | G |
| Example B29 | A | A | 24 | A | G |
| Example B30 | A | A | 25 | A | G |
| Example B31 | A | A | 26 | A | G |
| Example B32 | A | A | 25 | A | G |
| Example B33 | A | A | 24 | A | G |
| Example B34 | A | A | 27 | A | G |
| Example B35 | A | A | 26 | A | G |

### Example C

Evaluations of the EUV sensitivity and the etching defect were carried out in the same manner as in Example 15, except that the compound B1 and the compound B2 described in Table 8 were used instead of the compound 1-3 and the compound 1-3a in the following ratios.

TABLE 8

| Examples | Compound B1 | Ratio B1 | Compound B2 | Ratio B2 |
|---|---|---|---|---|
| Example C1 | 1-3 | 99.5 | DML1P | 0.5 |
| Example C2 | 1-3 | 99.5 | DML1R | 0.5 |
| Example C3 | 1-3 | 99.5 | BPL1P | 0.5 |
| Example C4 | 1-3 | 99.5 | BPL1R | 0.5 |
| Example C5 | 2-4 | 99.5 | BPN2-3P | 0.5 |
| Example C6 | 2-4 | 99.5 | DMN2-3P | 0.5 |
| Example C7 | Ad-2-2 | 99.5 | DMA2a | 0.5 |
| Example C8 | 3-2 | 99.5 | DMA1a | 0.5 |
| Example C9 | 6 | 99.5 | BPL1b | 0.5 |
| Example C10 | 1-4 | 99.5 | BPL2R | 0.5 |
| Example C11 | Na-2 | 99.5 | BPNa-2-1R | 0.5 |
| Example C12 | 2 | 99.5 | DML6R | 0.5 |
| Example C13 | 3 | 99.5 | DML2R | 0.5 |
| Example C14 | 4 | 99.5 | DML3R | 0.5 |
| Example C15 | 5 | 99.5 | DML4R | 0.5 |
| Example C16 | 1-7 | 99.5 | DML5R | 0.5 |
| Example C18 | 1-8 | 99.5 | DML7R | 0.5 |
| Example C19 | 1-5 | 99.5 | DML6R | 0.5 |
| Example C20 | 1-6 | 99.5 | DML8R | 0.5 |
| Example C21 | 1-4 | 99.5 | DML9R | 0.5 |
| Example C22 | 1-5 | 99.5 | DML6R | 0.5 |
| Example C24 | 1-7 | 99.5 | DML7R | 0.5 |
| Example C25 | 2-3 | 99.5 | DMN2-3 | 0.5 |
| Example C26 | Na-1 | 99.5 | DMNa-1-1 | 0.5 |
| Example C27 | Na-2 | 99.5 | DMNa-2-1R | 0.5 |
| Example C28 | Na-3 | 99.5 | DMNa-3-1R | 0.5 |
| Example C29 | Na-4-2 | 99.5 | DMNa-4-2 | 0.5 |
| Example C30 | Na-4-3 | 99.5 | DMNa-4-2P | 0.5 |
| Example C31 | Ad-A-1 | 99.5 | DMA1a | 0.5 |
| Example C32 | Ad-A-2 | 99.5 | DMA1aP2 | 0.5 |
| Example C34 | Ad-2-3 | 99.5 | DMA2aP2 | 0.5 |
| Example C35 | Ad-2-4 | 99.5 | DMA2aP | 0.5 |

TABLE 9

| Examples | EUV sensitivity (mJ/cm2) | Etching defect |
|---|---|---|
| Example C1 | 22 | S |
| Example C2 | 22 | S |
| Example C3 | 22 | S |
| Example C4 | 22 | S |
| Example C5 | 28 | S |
| Example C6 | 28 | S |
| Example C7 | 26 | S |
| Example C8 | 22 | S |
| Example C9 | 24 | S |
| Example C10 | 27 | S |
| Example C11 | 26 | S |
| Example C12 | 27 | S |
| Example C13 | 25 | S |
| Example C14 | 24 | S |
| Example C15 | 27 | S |
| Example C16 | 26 | S |
| Example C18 | 25 | S |
| Example C19 | 26 | S |
| Example C20 | 25 | S |
| Example C21 | 24 | S |
| Example C22 | 26 | S |
| Example C24 | 26 | S |
| Example C25 | 27 | S |
| Example C26 | 24 | S |
| Example C27 | 27 | S |
| Example C28 | 26 | S |
| Example C29 | 24 | S |
| Example C30 | 25 | S |
| Example C31 | 26 | S |
| Example C32 | 25 | S |
| Example C34 | 27 | S |
| Example C35 | 26 | S |

### Example D

Compounds subjected to treatment 1 or treatment 2 were obtained in the same manner as in Example 12, except that the compounds shown in Table 10 were used instead of the compound 3-2, and evaluations of the EUV sensitivity and the etching defect were carried out. As a result, as in Example 12, good results were confirmed for each compound with respect to the EUV sensitivity and the etching defect.

TABLE 10
Examples D1 to D35

| Examples | Compound |
|---|---|
| Example D1 | 1-3 |
| Example D5 | 2-4 |
| Example D7 | Ad-2-2 |
| Example D8 | 3-2 |
| Example D9 | 6 |
| Example D10 | 1-4 |
| Example D11 | Na-2 |
| Example D12 | 2 |
| Example D13 | 3 |
| Example D14 | 4 |
| Example D15 | 5 |
| Example D16 | 1-7 |
| Example D18 | 1-8 |
| Example D19 | 1-5 |
| Example D20 | 1-6 |
| Example D21 | 1-4 |
| Example D25 | 2-3 |
| Example D26 | Na-1 |
| Example D27 | Na-2 |
| Example D28 | Na-3 |
| Example D29 | Na-4-2 |
| Example D30 | Na-4-3 |
| Example D31 | Ad-A-1 |
| Example D32 | Ad-A-2 |
| Example D34 | Ad-2-3 |
| Example D35 | Ad-2-4 |

TABLE 11

| | Treatment 1 | | Treatment 2 | |
|---|---|---|---|---|
| | (purification by acid) | | (treatment without acid) | |
| | EUV sensitivity | Etching | EUV sensitivity | Etching |
| Examples | (mJ/cm²) | defect | (mJ/cm²) | defect |
| Example D1 | 22 | A | 25 | B |
| Example D5 | 28 | A | 24 | B |
| Example D7 | 26 | A | 26 | B |
| Example D8 | 22 | A | 25 | B |
| Example D9 | 24 | A | 24 | B |
| Example D10 | 27 | A | 27 | B |
| Example D11 | 26 | A | 26 | B |
| Example D12 | 27 | A | 27 | B |
| Example D13 | 25 | A | 25 | B |
| Example D14 | 24 | A | 24 | B |
| Example D15 | 27 | A | 27 | B |
| Example D16 | 26 | A | 26 | B |
| Example D18 | 25 | A | 25 | B |
| Example D19 | 26 | A | 26 | B |
| Example D20 | 25 | A | 25 | B |
| Example D21 | 24 | A | 24 | B |
| Example D25 | 27 | A | 25 | B |
| Example D26 | 24 | A | 24 | B |
| Example D27 | 27 | A | 27 | B |
| Example D28 | 26 | A | 26 | B |
| Example D29 | 24 | A | 24 | B |
| Example D30 | 25 | A | 25 | B |
| Example D31 | 26 | A | 26 | B |
| Example D32 | 25 | A | 25 | B |
| Example D34 | 27 | A | 27 | B |
| Example D35 | 26 | A | 26 | B |

### Example 101B

The following compositions were prepared according to the method of Example 4 (numerical value: parts by mass).

TABLE 12
Example 101B (composition of composition)

| | | parts | | parts | | parts |
|---|---|---|---|---|---|---|
| Composition | Compound | by | Compound | by | | by |
| name | B1 | mass | B2 | mass | Solvent | mass |
| L1-BP1 | 1-3 | 400 | BPL1P | 1 | Toluene | 400 |
| L1-BP2 | 1-3 | 400 | BPL1P | 0.1 | Toluene | 400 |
| L1-BP3 | 1-3 | 400 | BPL1P | 0.01 | Toluene | 400 |
| L1-BP4 | 1-3 | 400 | BPL1P | 0.005 | Toluene | 400 |
| L1-BP5 | 1-3 | 400 | BPL1P | 0.001 | Toluene | 400 |
| L1-BR1 | 1-3 | 400 | BPL1R | 1 | Toluene | 400 |
| L1-BR2 | 1-3 | 400 | BPL1R | 0.1 | Toluene | 400 |
| L1-BR3 | 1-3 | 400 | BPL1R | 0.01 | Toluene | 400 |
| L1-BR4 | 1-3 | 400 | BPL1R | 0.005 | Toluene | 400 |
| L1-BR5 | 1-3 | 400 | BPL1R | 0.001 | Toluene | 400 |
| L1-NA | 1-3 | 400 | — | — | Toluene | 400 |

For the above compositions, the aging test was carried out under the following conditions, and the state of the solution after the test was evaluated by the absorbance using a spectrophotometer. Specifically, the spectrum in the visible light region was measured for samples after the aging test, “the average value Al of each absorbance at 450 nm, 550 nm, and 650 nm” was determined, and the difference ΔA with “the average value A0 of each absorbance at 450 nm, 550 nm, and 650 nm” before initiation of the test was calculated and evaluated.

$$Δ A = A 1 - A 0$$

TABLE 13
(Evaluation of composition: ΔA value)

| | | | Condition 3 | Condition 4 |
|---|---|---|---|---|
| | | | N2 purge | Air purge |
| | Condition 1 | Condition 2 | 1% moisture | 1% moisture |
| | N2 purge | Air purge | content | content |
| Composition | 40° C. | 40° C. | 40° C. | 40° C. |
| name | 1 month | 1 month | 1 month | 1 month |
| L1-BP1 | 0.1 | 0.2 | 0.1 | 0.2 |
| L1-BP2 | 0.04 | 0.08 | 0.04 | 0.09 |
| L1-BP3 | 0.01 | 0.01 | 0.01 | 0.01 |
| L1-BP4 | ≤0.01 | ≤0.01 | ≤0.01 | ≤0.01 |
| L1-BP5 | 0.01 | 0.01 | 0.01 | 0.01 |
| L1-BR1 | 0.1 | 0.2 | 0.1 | 0.2 |
| L1-BR2 | 0.04 | 0.08 | 0.04 | 0.09 |
| L1-BR3 | 0.01 | 0.01 | 0.01 | 0.01 |
| L1-BR4 | ≤0.01 | ≤0.01 | ≤0.01 | ≤0.01 |
| L1-BR5 | 0.01 | 0.01 | 0.01 | 0.01 |
| L1-NA | 0.3 | 0.5 | 0.3 | 0.6 |

As a result, it was found that, in each of the compositions, the increase of the absorbance in the spectroscopic spectrum after the aging test is suppressed by using a predetermined amount of the compound B2 in combination. The compositions in which a predetermined amount of the compound B2 was used in combination had a low ΔA value as compared with that of L1-NA in which only one kind of compound was used. It was found from these results that the stability with time is improved by using these compositions.

### Example 101D

The following compositions were prepared according to the method of Example 4 (numerical value: parts by mass). The stability with time of the compositions was evaluated in the same manner as in Example 101B.

TABLE 14
Example 101D (composition of composition)

| | | parts | | parts | | parts |
|---|---|---|---|---|---|---|
| Composition | Compound | by | Compound | by | | by |
| name | B1 | mass | B2 | mass | Solvent | mass |
| L1-DM1 | 1-3 | 400 | DML1D | 1 | Toluene | 400 |
| L1-DM2 | 1-3 | 400 | DML1D | 0.1 | Toluene | 400 |
| L1-DM3 | 1-3 | 400 | DML1D | 0.01 | Toluene | 400 |
| L1-DM4 | 1-3 | 400 | DML1D | 0.005 | Toluene | 400 |
| L1-DM5 | 1-3 | 400 | DML1D | 0.001 | Toluene | 400 |
| L1-DP1 | 1-3 | 400 | DML1P | 1 | Toluene | 400 |
| L1-DP2 | 1-3 | 400 | DML1P | 0.1 | Toluene | 400 |
| L1-DP3 | 1-3 | 400 | DML1P | 0.01 | Toluene | 400 |
| L1-DP4 | 1-3 | 400 | DML1P | 0.005 | Toluene | 400 |
| L1-DP5 | 1-3 | 400 | DML1P | 0.001 | Toluene | 400 |
| L1-DR1 | 1-3 | 400 | DML1R | 1 | Toluene | 400 |
| L1-DR2 | 1-3 | 400 | DML1R | 0.1 | Toluene | 400 |
| L1-DR3 | 1-3 | 400 | DML1R | 0.01 | Toluene | 400 |
| L1-DR4 | 1-3 | 400 | DML1R | 0.005 | Toluene | 400 |
| L1-DR5 | 1-3 | 400 | DML1R | 0.001 | Toluene | 400 |
| L1-NA | 1-3 | 400 | — | — | Toluene | 400 |

TABLE 15
(Evaluation of composition: ΔA value)

| | | | Condition 3 | Condition 4 |
|---|---|---|---|---|
| | | | N2 purge | Air purge |
| | Condition 1 | Condition 2 | 1% moisture | 1% moisture |
| | N2 purge | Air purge | content | content |
| Composition | 40° C. | 40° C. | 40° C. | 40° C. |
| name | 1 month | 1 month | 1 month | 1 month |
| L1-DM1 | 0.1 | 0.2 | 0.1 | 0.2 |
| L1-DM2 | 0.04 | 0.08 | 0.04 | 0.09 |
| L1-DM3 | 0.01 | 0.01 | 0.01 | 0.01 |
| L1-DM4 | ≤0.01 | ≤0.01 | ≤0.01 | ≤0.01 |
| L1-DM5 | 0.01 | 0.01 | 0.01 | 0.01 |
| L1-DP1 | 0.1 | 0.2 | 0.1 | 0.2 |
| L1-DP2 | 0.04 | 0.08 | 0.04 | 0.09 |
| L1-DP3 | 0.01 | 0.01 | 0.01 | 0.01 |
| L1-DP4 | ≤0.01 | ≤0.01 | ≤0.01 | ≤0.01 |
| L1-DP5 | 0.01 | 0.01 | 0.01 | 0.01 |
| L1-DR1 | 0.1 | 0.2 | 0.1 | 0.2 |
| L1-DR2 | 0.04 | 0.08 | 0.04 | 0.09 |
| L1-DR3 | 0.01 | 0.01 | 0.01 | 0.01 |
| L1-DR4 | ≤0.01 | ≤0.01 | ≤0.01 | ≤0.01 |
| L1-DR5 | 0.01 | 0.01 | 0.01 | 0.01 |
| L1-NA | 0.3 | 0.5 | 0.3 | 0.6 |

As a result, it was found that, in each of the compositions, the increase of the absorbance in the spectroscopic spectrum after the aging test is suppressed by using a predetermined amount of the compound 2 in combination. It was found from these results that the stability with time is improved by using these compositions.

### Example 102B

The following compositions were prepared according to the method of Example 4 (numerical value: parts by mass). The stability with time of the compositions was evaluated in the same manner as in Example 101B.

TABLE 16
Example 102B (composition of composition)

| | | parts | | parts | | parts |
|---|---|---|---|---|---|---|
| Composition | Compound | by | Compound | by | | by |
| name | B1 | mass | B2 | mass | Solvent | mass |
| L2-BP1 | 2-4 | 400 | BPN2-3P | 1 | Toluene | 400 |
| L2-BP2 | 2-4 | 400 | BPN2-3P | 0.1 | Toluene | 400 |
| L2-BP3 | 2-4 | 400 | BPN2-3P | 0.01 | Toluene | 400 |
| L2-BP4 | 2-4 | 400 | BPN2-3P | 0.005 | Toluene | 400 |
| L2-BP5 | 2-4 | 400 | BPN2-3P | 0.001 | Toluene | 400 |
| L2-NA | 2-4 | 400 | — | — | Toluene | 400 |

TABLE 17
(Evaluation of composition: ΔA value)

| | | | Condition 3 | Condition 4 |
|---|---|---|---|---|
| | | | N2 purge | Air purge |
| | Condition 1 | Condition 2 | 1% moisture | 1% moisture |
| | N2 purge | Air purge | content | content |
| Composition | 40° C. | 40° C. | 40° C. | 40° C. |
| name | 1 month | 1 month | 1 month | 1 month |
| L2-BP1 | 0.1 | 0.2 | 0.1 | 0.2 |
| L2-BP2 | 0.04 | 0.08 | 0.04 | 0.09 |
| L2-BP3 | 0.01 | 0.01 | 0.01 | 0.01 |
| L2-BP4 | ≤0.01 | ≤0.01 | ≤0.01 | ≤0.01 |
| L2-BP5 | 0.01 | 0.01 | 0.01 | 0.01 |
| L2-NA | 0.3 | 0.5 | 0.3 | 0.6 |

As a result, it was found that, in each of the compositions, the increase of the absorbance in the spectroscopic spectrum after the aging test is suppressed by using a predetermined amount of the compound 2 in combination. It was found from these results that the stability with time is improved by using these compositions.

### Example 102D

The following compositions were prepared according to the method of Example 4 (numerical value: parts by mass). The stability with time of the compositions was evaluated in the same manner as in Example 101B.

TABLE 18
Example 102D (composition of composition)

| Composi- | Com- | | Com- | | | |
|---|---|---|---|---|---|---|
| tion | pound | | pound | | | |
| name | B1 | Amount | B2 | Amount | Solvent | Amount |
| L2-DP1 | 2-4 | 400 | DMN2-3P | 1 | Toluene | 400 |
| L2-DP2 | 2-4 | 400 | DMN2-3P | 0.1 | Toluene | 400 |
| L2-DP3 | 2-4 | 400 | DMN2-3P | 0.01 | Toluene | 400 |
| L2-DP4 | 2-4 | 400 | DMN2-3P | 0.005 | Toluene | 400 |
| L2-DP5 | 2-4 | 400 | DMN2-3P | 0.001 | Toluene | 400 |
| L2-NA | 2-4 | 400 | — | — | Toluene | 400 |

TABLE 19
(Evaluation of composition: ΔA value)

| | | | Condition 3 | Condition 4 |
|---|---|---|---|---|
| | | | N2 purge | Air purge |
| | Condition 1 | Condition 2 | 1% moisture | 1% moisture |
| | N2 purge | Air purge | content | content |
| Composition | 40° C. | 40° C. | 40° C. | 40° C. |
| name | 1 month | 1 month | 1 month | 1 month |
| L2-DP1 | 0.1 | 0.2 | 0.1 | 0.2 |
| L2-DP2 | 0.04 | 0.08 | 0.04 | 0.09 |
| L2-DP3 | 0.01 | 0.01 | 0.01 | 0.01 |
| L2-DP4 | ≤0.01 | ≤0.01 | ≤0.01 | ≤0.01 |
| L2-DP5 | 0.01 | 0.01 | 0.01 | 0.01 |
| L2-NA | 0.3 | 0.5 | 0.3 | 0.6 |

As a result, it was found that, in each of the compositions, the increase of the absorbance in the spectroscopic spectrum after the aging test is suppressed by using a predetermined amount of the compound 2 in combination. It was found from these results that the stability with time is improved by using these compositions.

### Example 103B

The following compositions were prepared according to the method of Example 4 (numerical value: parts by mass). The stability with time of the compositions was evaluated in the same manner as in Example 101B.

TABLE 20
Example 103B (composition of composition)

| Composi- | Com- | | Com- | | | |
|---|---|---|---|---|---|---|
| tion | pound | | pound | | | |
| name | B1 | Amount | B2 | Amount | Solvent | Amount |
| L3-BP1 | Ad-2-2 | 400 | DMA2a | 1 | Toluene | 400 |
| L3-BP2 | Ad-2-2 | 400 | DMA2a | 0.1 | Toluene | 400 |
| L3-BP3 | Ad-2-2 | 400 | DMA2a | 0.01 | Toluene | 400 |
| L3-BP4 | Ad-2-2 | 400 | DMA2a | 0.005 | Toluene | 400 |
| L3-BP5 | Ad-2-2 | 400 | DMA2a | 0.001 | Toluene | 400 |
| L3-NA | Ad-2-2 | 400 | — | — | Toluene | 400 |

TABLE 21
(Evaluation of composition: ΔA value)

| | | | Condition 3 | Condition 4 |
|---|---|---|---|---|
| | | | N2 purge | Air purge |
| | Condition 1 | Condition 2 | 1% moisture | 1% moisture |
| | N2 purge | Air purge | content | content |
| Composition | 40° C. | 40° C. | 40° C. | 40° C. |
| name | 1 month | 1 month | 1 month | 1 month |
| L3-BP1 | 0.1 | 0.2 | 0.1 | 0.2 |
| L3-BP2 | 0.04 | 0.08 | 0.04 | 0.09 |
| L3-BP3 | 0.01 | 0.01 | 0.01 | 0.01 |
| L3-BP4 | ≤0.01 | ≤0.01 | ≤0.01 | ≤0.01 |
| L3-BP5 | 0.01 | 0.01 | 0.01 | 0.01 |
| L3-NA | 0.3 | 0.5 | 0.3 | 0.6 |

As a result, it was found that, in each of the compositions, the increase of the absorbance in the spectroscopic spectrum after the aging test is suppressed by using a predetermined amount of the compound 2 in combination. It was found from these results that the stability with time is improved by using these compositions.

### Example 103D

The following compositions were prepared according to the method of Example 4 (numerical value: parts by mass). The stability with time of the compositions was evaluated in the same manner as in Example 101B.

TABLE 22
Example 103D (composition of composition)

| Composi- | Com- | | Com- | | | |
|---|---|---|---|---|---|---|
| tion | pound | | pound | | | |
| name | B1 | Amount | B2 | Amount | Solvent | Amount |
| L3-DP1 | 3-2 | 400 | DMA1a | 1 | Toluene | 400 |
| L3-DP2 | 3-2 | 400 | DMA1a | 0.1 | Toluene | 400 |
| L3-DP3 | 3-2 | 400 | DMA1a | 0.01 | Toluene | 400 |
| L3-DP4 | 3-2 | 400 | DMA1a | 0.005 | Toluene | 400 |
| L3-DP5 | 3-2 | 400 | DMA1a | 0.001 | Toluene | 400 |
| L3-NA | 3-2 | 400 | — | — | Toluene | 400 |

TABLE 23
(Evaluation of composition: ΔA value)

| | | | Condition 3 | Condition 4 |
|---|---|---|---|---|
| | | | N2 purge | Air purge |
| | Condition 1 | Condition 2 | 1% moisture | 1% moisture |
| | N2 purge | Air purge | content | content |
| Composition | 40° C. | 40° C. | 40° C. | 40° C. |
| name | 1 month | 1 month | 1 month | 1 month |
| L3-DP1 | 0.1 | 0.2 | 0.1 | 0.2 |
| L3-DP2 | 0.04 | 0.08 | 0.04 | 0.09 |
| L3-DP3 | 0.01 | 0.01 | 0.01 | 0.01 |
| L3-DP4 | ≤0.01 | ≤0.01 | ≤0.01 | ≤0.01 |
| L3-DP5 | 0.01 | 0.01 | 0.01 | 0.01 |
| L3-NA | 0.3 | 0.5 | 0.3 | 0.6 |

As a result, it was found that, in each of the compositions, the increase of the absorbance in the spectroscopic spectrum after the aging test is suppressed by using a predetermined amount of the compound 2 in combination. It was found from these results that the stability with time is improved by using these compositions.

### Examples 104B to 106B

The aging test was carried out in the same manner as in Example 101B, except that the compound B1 and the compound B2 in Example 101B were changed to the compounds described in the following Table. As a result, similar results with Example 101B were obtained.

TABLE 24
Examples 104B to 106B (composition of composition)

| Examples | Compound B1 | Compound B2 |
|---|---|---|
| 104B | 6 | BPL1b |
| 105B | 1-4 | BPL2R |
| 106B | Na-2 | BPNa-2-1R |

The aging test was carried out in the same manner as in Example 101D, except that the compound B1 and the compound B2 in Example 101D were changed to the compounds described in the following Table. As a result, similar results with Example 101D were obtained.

TABLE 25
Examples 104D to 127D (composition of composition)

| Examples | Compound B1 | Compound B2 |
|---|---|---|
| 104D | 2 | DML6R |
| 105D | 3 | DML2R |
| 106D | 4 | DML3R |
| 107D | 5 | DML4R |
| 110D | 1-8 | DML7R |
| 113D | 1-4 | DML9R |
| 114D | 1-5 | DML6R |
| 115D | 1-6 | DML8R |
| 116D | 1-7 | DML5R |
| 117D | 2-3 | DMN2-3 |
| 118D | Na-1 | DMNa-1-1 |
| 119D | Na-2 | DMNa-2-1R |
| 120D | Na-3 | DMNa-3-1R |
| 121D | Na-4-2 | DMNa-4-2 |
| 122D | Na-4-3 | DMNa-4-2P |
| 123D | Ad-A-1 | DMA1a |
| 124D | Ad-A-2 | DMA1aP2 |
| 125D | Ad-2-2 | DMA2a |
| 126D | Ad-2-3 | DMA2aP2 |
| 127D | Ad-2-4 | DMA2aP |

As a result, it was found that, in each of the above compositions, the increase of the absorbance in the spectroscopic spectrum after the aging test is suppressed by using a predetermined amount of the compound 2 in combination. It was found from these results that the stability with time is improved by using these compositions.

1. A compound represented by the following formula (1): 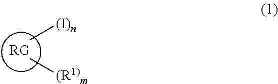
wherein RG is a group containing at least one cyclic structure;
I is an iodine atom;
R¹ is a monovalent functional group having 0 to 30 carbon atoms, containing no polymerizable unsaturated bond, and being optionally the same or different;
n is an integer of 1 to 5; and
m is an integer of 1 to 5.

2. A composition for lithography comprising the compound according to claim 1.

3. The composition for lithography according to claim 2 comprising two or more kinds of compounds represented by the formula (1).

4. A composition comprising the compound according to claim 1.

5. The composition according to claim 4, further comprising a compound represented by the following formula (DM0-1), the following formula (BP0-1), or a combination thereof: 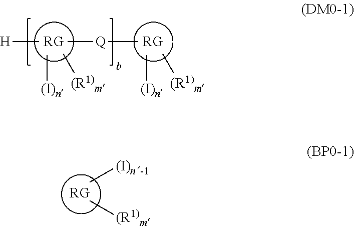
wherein RG, I, and R¹ are as defined in the formula (1);
Q is a group resulting from a group bonding molecules, or a single bond;
n′ is an integer of 1 to 5 and n or less;
m′ is an integer of 1 to 5 and m or less; and
b is an integer of 1 to 4.

6. The composition according to claim 5, wherein the compound represented by the formula (DM0-1) is a compound represented by the following formula (DM1a), (Dn1), or (Da1): 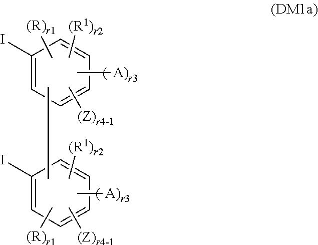
wherein Z is I, R¹, H, or a linking group for forming a dimer;
R¹ is as defined in the formula (1);
A is a group having a protective group;
R is a hydrogen atom or an organic group other than a functional group;
R¹, A, and R are each bonded to a bondable position; and
r1 to r4 are an integer of 0 to 5, and a sum of r1 to r4 in a benzene ring satisfies a valence of the benzene ring, provided that at least one of r2 and r3 is selected so as to be 1 or more; 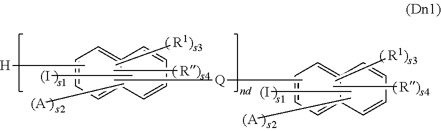
wherein I, R¹, and A are as defined in the formula (DM1a);
R″ is a hydrogen atom or an organic group other than R¹;
I, R¹, A, and R″ are each bonded to a bondable position;
Q is as defined in the formula (DM0-1);
s1 is an integer of 1 to 7, and s2 to s4 are an integer of 0 to 7, provided that s1 to s4 are selected so that a sum of s1 to s4 satisfies a valence of a naphthalene ring, and either one of s2 and s3 is 1 or more; and
nd is an integer of 1 to 4; and 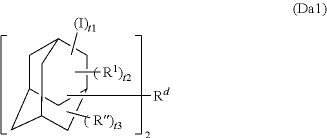
wherein I and R¹ are as defined in the formula (Dn1);
R″ is a hydrogen atom or an organic group other than R¹;
R^{d} is a single bond or —O— (an ether bond); and
t1 is an integer of 1 to 10, t2 is an integer of 1 to 9, and t3 is an integer of 0 to 14, provided that t1 to t3 are selected so that a sum of t1 to t3 satisfies a valence of an adamantane ring, and
the compound represented by the formula (BP0-1) is a compound represented by the following formula (BP1a), (BP2a), (Bn1), or (Ba1): 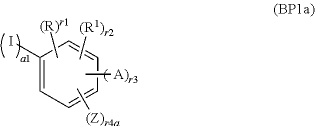
wherein Z, R, R¹, and A are as defined in the formula (DM1a);
r1, r2, and r3 are an integer of 0 to 5;
a1 and r4a are an integer of 0 to 4; and
a1 and r4a satisfy a1+r4a≤r4, provided that r4 is as defined in the formula (DM1a); 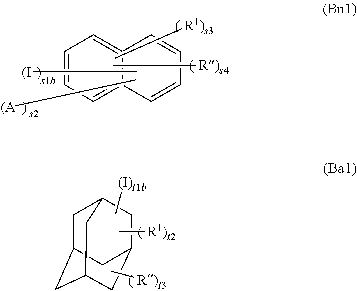
wherein R¹, R″, and A are as defined in the formulas (Dn1) and (Da1);
s2 to s4 are as defined in the formula (Dn1);
s1b is an integer of 0 to 6, and is an integer satisfying s1b≤(s1−1), wherein si is as defined in the formulas (Dn1) and (Da1);
t2 and t3 are as defined in (Da1); and
t1b is an integer of 0 to 9, and is an integer satisfying t1b≤(t1−1), wherein ti is as defined in the formula (Da1).

7. The composition according to claim 5, comprising the compound represented by the formula (DM0-1).

8. The composition of claim 7, wherein the compounds represented by the formula (1) and the formula (DM0-1) satisfy the following relationship:
0.1≥[an amount of the compound of the formula(DM0-1)]/[an amount of the compound of the formula(1)]≥0.000001.

9. The composition according to claim 8, wherein the compound represented by the formula (DM0-1) is a compound represented by the formula (DM1a), the formula (Dn1), or the formula (Da1).

10. The composition according to claim 5, comprising the compound represented by the formula (BP0-1).

11. The composition according to claim 10, wherein the compound represented by the formula (BP0-1) is a compound represented by the formula (BP1a), the formula (BP2a), the formula (Bn1), or the formula (Ba1).

12. The composition according to claim 10, wherein the compound represented by the formula (BP0-1) is a compound represented by the formula (BP1a) wherein Z is not I, or a compound represented by the formula (BP2a), the formula (Bn1), or the formula (Ba1).

13. The composition according to claim 5, wherein the compounds represented by the formula (1), the formula (DM0-1), and the formula (BP0-1) satisfy the following relational expression:
0.1≥([a total amount of the compound of the formula(DM0-1) and the compound of the formula(BP0-1)])/[an amount of the compound of the formula(1)]≥0.000001.

14. The composition for lithography according to claim 2, wherein RG in the formula (1) is a group derived from a benzene ring optionally having a substituent.

15. The composition for lithography according to claim 2, wherein RG in the formula (1) is a group derived from a naphthalene ring optionally having a substituent.

16. The composition for lithography according to claim 2, wherein RG in the formula (1) is a group derived from an adamantane ring optionally having a substituent.

17. The composition according to claim 4, wherein a content of metal impurities is less than 1 ppm.

18. A method for producing the compound according to claim 1, comprising a step of introducing an iodine atom or an R¹ group into the compound containing the RG group.

19. A method for producing the compound according to claim 1, that is a method for producing the compound represented by the formula (1), wherein
the compound is represented by the formula (Bz): 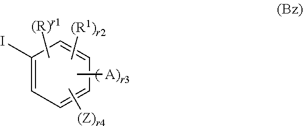
wherein Z, R¹, A, R, and r1 to r4 are as defined in the formula (DM1a), the method comprising:
1) a step of providing a compound represented by the formula (MB): 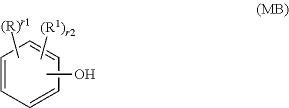
wherein R¹, R, r1, and r2 are as defined in the formula (Bz), and R¹, R, and OH are each bonded to any bondable position;
2) an iodination step of iodinating the compound;
3) a protective group introduction step of introducing a protective group into the compound; and
4) a reduction step of reducing the compound.

20. A method for producing the compound according to claim 1, that is a method for producing the compound represented by the formula (1), wherein
the compound is represented by the formula (N): 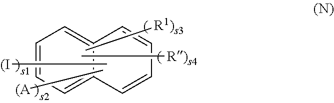
wherein I, R¹, A, and R″ are as defined in the formulas (Dn1) and (Bn1);
provided that I, R¹, R″, and A are each bonded to any bondable position; and
s1 is an integer of 1 to 7, and s2 to s4 are an integer of 0 to 7, provided that s1 to s4 are selected so that a sum of s1 to s4 satisfies a valence of a naphthalene ring and either one of s2 and s3 is 1 or more, the method comprising:
1) a step of providing a compound represented by the formula (MN): 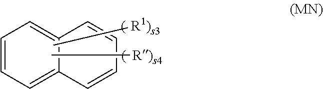
wherein R¹, R″, s3, and s4 are as defined in the formula (N);
2) an iodination step of iodinating the compound;
3) a protective group introduction step of introducing a protective group into the compound; and
4) a reduction step of reducing the compound.

21. A method for producing the compound according to claim 1, that is a method for producing the compound represented by the formula (1), wherein
the compound is represented by the formula (Ad): 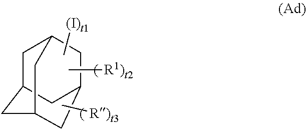
wherein R¹ and R″ are as defined in the formula (Da1);
I, R¹, and R″ are each bonded to any bondable position; and
t1 is an integer of 1 to 10, t2 is an integer of 1 to 9, and t3 is an integer of 0 to 14, provided that t1 to t3 are selected so that a sum of t1 to t3 satisfies a valence of an adamantane ring, the method comprising:
1) a step of providing a compound represented by the formula (MA): 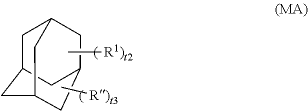
wherein R¹, R, t2, and t3 are as defined in the formula (Ad); and
2) an iodination step of iodinating the compound.

22. A method for expressing a sensitizing effect in radiation irradiation of a composition for lithography using the compound according to claim 1.

23. The method according to claim 22, wherein two or more kinds of the compounds are used.

24. The compound according to claim 1, wherein
RG is a group derived from benzene, naphthalene, anthracene, pyrene, a heteroaromatic compound, or a polycyclic alicyclic compound, optionally having a substituent; and
R¹ is composed of
one or more R^{f′} selected from the group consisting of a hydroxy group, and an ether group having a protective group eliminatable by acid, alkali, or heat, and
zero or more hydrocarbon groups R^{g} having 0 to 30 carbon atoms and optionally containing a substituent.

25. The compound according to claim 1, wherein
RG is a group derived from benzene or naphthalene, the group optionally having a substituent; and
R¹ is composed of
one or more R^{f} selected from the group consisting of a hydroxy group, and an ether group having a protective group, and
zero or more hydrocarbon groups R^{g} having 0 to 30 carbon atoms and optionally containing a substituent.

26. The compound according to claim 1, wherein
RG is a group derived from a polycyclic alicyclic compound optionally having a substituent; and
R¹ is composed of
one or more R^{f′} selected from the group consisting of a hydroxy group, and an ether group having a protective group eliminatable by acid, alkali, or heat, and
zero or more hydrocarbon groups R^{g} having 0 to 30 carbon atoms and optionally containing a substituent.

27. The compound according to claim 1, wherein
RG is a group derived from a polycyclic alicyclic compound optionally having a substituent; and
R¹ is composed of
one or more R^{f} selected from the group consisting of a hydroxy group, and an ether group having a protective group, and
zero or more hydrocarbon groups R^{g} having 0 to 30 carbon atoms and optionally containing a substituent.

28. The compound according to claim 1, wherein
when RG is a group containing a benzene ring and a plurality of R¹ is present, R¹ contains no combination of an alkoxy group (excluding one having a protective group) and an aldehyde group, no combination of the alkoxy group and a hydroxy group, and no combination of an aldehyde group and a hydroxy group, and
when RG is a group containing a naphthalene ring and a plurality of R¹ is present, R¹ contains no combination of a hydroxy group and a carboxyl group.

29. The compound according to claim 1, wherein
R¹ is selected from —OR², —COOR³, —CH₂—OR⁴, or —CHO,
wherein
R² is a hydrogen atom, an alkyl group having 1 to 30 carbon atoms and optionally having a substituent, or an aryl group having 1 to 30 carbon atoms and optionally having a substituent;
R³ is a hydrogen atom, an alkyl group having 1 to 29 carbon atoms and optionally having a substituent, or an aryl group having 1 to 29 carbon atoms and optionally having a substituent; and
R⁴ is a hydrogen atom, an alkyl group having 1 to 29 carbon atoms and optionally having a substituent, or an aryl group having 1 to 29 carbon atoms and optionally having a substituent.

30. The compound according to claim 1, wherein R¹ has a protective group.

31. The compound according to claim 1, wherein RG is a group derived from benzene optionally having a substituent, naphthalene optionally having a substituent, anthracene optionally having a substituent, phenanthrene optionally having a substituent, pyrene optionally having a substituent, fluorene optionally having a substituent, or adamantane optionally having a substituent.

32. The compound according to claim 31, wherein RG is a group derived from benzene optionally having a substituent, naphthalene optionally having a substituent, or adamantane optionally having a substituent.

33. The compound according to claim 32, wherein RG is a group derived from benzene optionally having a substituent.

34. The compound according to claim 32, wherein RG is a group derived from naphthalene optionally having a substituent.

35. The compound according to claim 32, wherein RG is a group derived from adamantane optionally having a substituent.

36. The compound according to claim 1, wherein the compound is represented by the formula (Bz), the formula (N), or the formula (Ad): 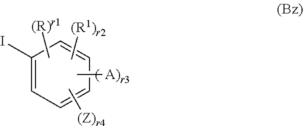
wherein I, Z, R¹, A, R, and r1 to r4 are as defined above, 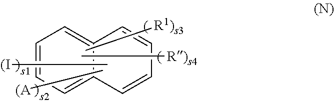
wherein I, R¹, R″, and s1 to s4 are as defined above, and 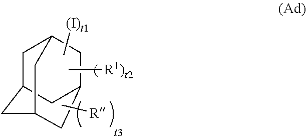
wherein I, R¹, R″, and t1 to t3 are as defined above.

37. The compound according to claim 36, wherein the compound is represented by any one of the following formulas: 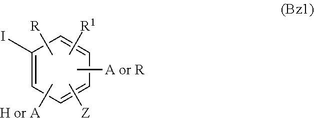
wherein Z, R, R¹, and A are as defined in the formula (Bz), 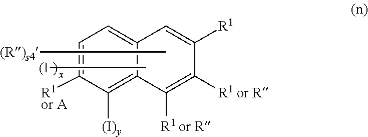
wherein R¹, A, and R″ are as defined in the formula (N);
x and y are 0 or 1, provided that at least either one is 1; and
s4′ represents a number of R″ capable of bonding to 1-, 7-, and 8-positions of a naphthalene ring, and is an integer of 1 to 3, and 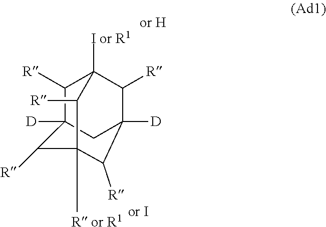
wherein I, R¹, and R″ are as defined in the formula (Ad),
one D is I, and the other D is R¹.

38. The compound according to claim 37, wherein the compound is represented by any one of the following formulas: 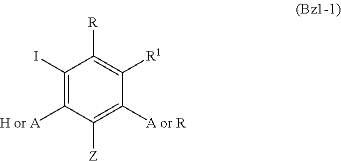
wherein I, Z, R, R¹, and A are as defined in the formula (Bz), 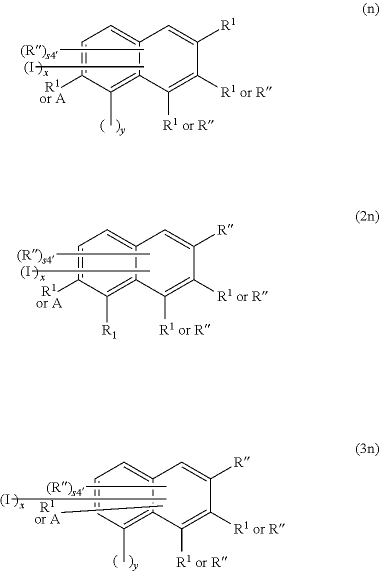
wherein I, Z, R¹, R″, A, x, y, and s4′ are as defined above, and 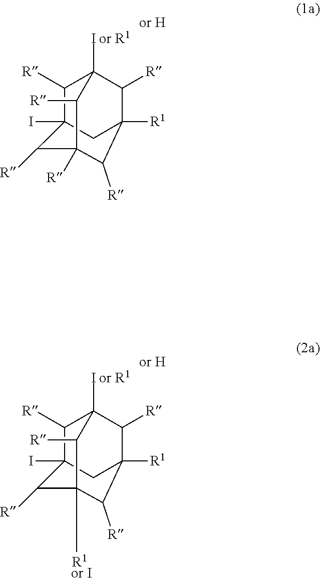
wherein I, R¹, and R″ are as defined in the formula (Ad).

39. The compound according to claim 33, wherein the compound is represented by any one of the following formulas: 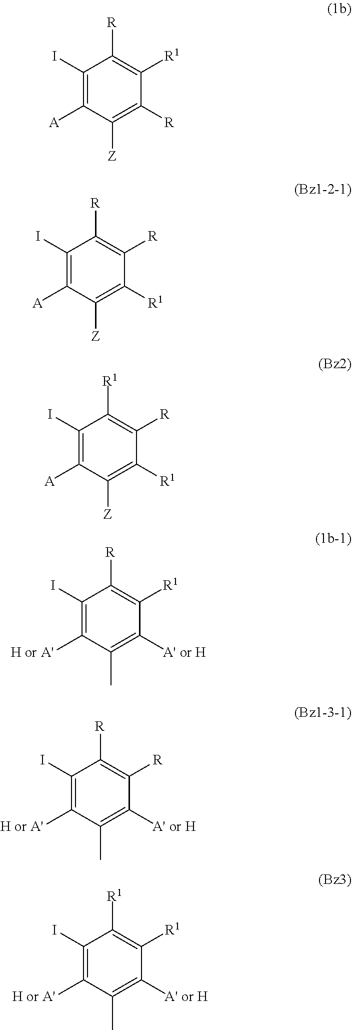
wherein I, R, and R¹ are as defined in the formula (Bz);
A′ is a group having a protective group, and represented by —O—R^{a}—O—R^{b}, —O—CO—O—R^{b}, or —O—R^{a}—CO—O—R^{b};
R^{a} is a linear or branched alkyl group having 1 to 3 carbon atoms; and R^{b} is a monovalent linear, branched, or cyclic alkyl group having 1 to 3 carbon atoms or a divalent cyclic alkyl group, and forms a ring with an adjacent oxygen atom.

40. The compound according to claim 34, wherein the compound is represented by any one of the following formulas: 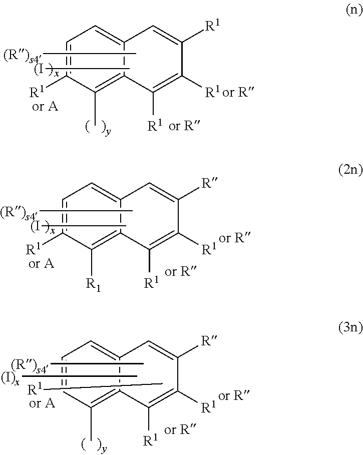
wherein I, Z, R¹, R″, A, x, y, and s4′ are as defined above.

41. The compound according to claim 40, wherein the compound is represented by any one of the following formulas: 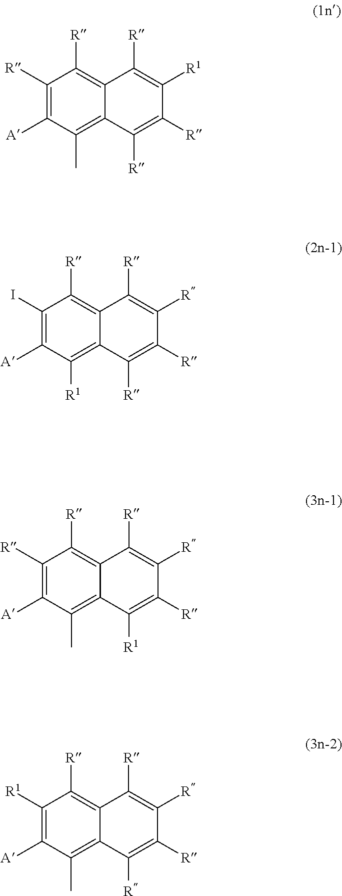
wherein I, R¹, R″, and A′ are as defined in the formula (n);
A′ is a group having a protective group, and represented by —O—R^{a}—O—R^{b}, —O—CO—O—R^{b}, or —O—R^{a}—CO—O—R^{b};
R^{a} is a linear or branched alkyl group having 1 to 3 carbon atoms; and R^{b} is a monovalent linear, branched, or cyclic alkyl group having 1 to 3 carbon atoms or a divalent cyclic alkyl group, and forms a ring with an adjacent oxygen atom.

42. The compound according to claim 35, wherein the compound is represented by any one of the following formulas: 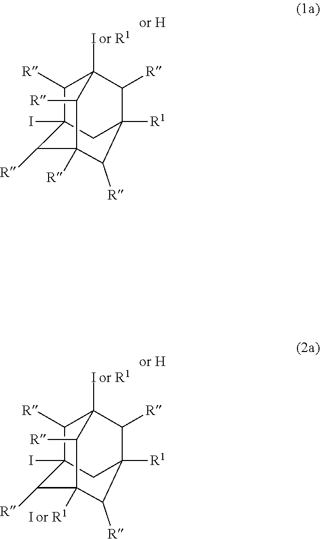
wherein I, R¹, and R″ are as defined in the formula (Ad).

43. The compound according to claim 36, wherein R¹ is a hydroxy group, a carboxyl group, an ester group, or a hydroxyalkyl group, and
when A is A′ represented by —O—R^{a}—O—R^{b}, the compound contains one or more A′.

44. The compound according to claim 43, wherein the compound is represented by any one of the following formulas: 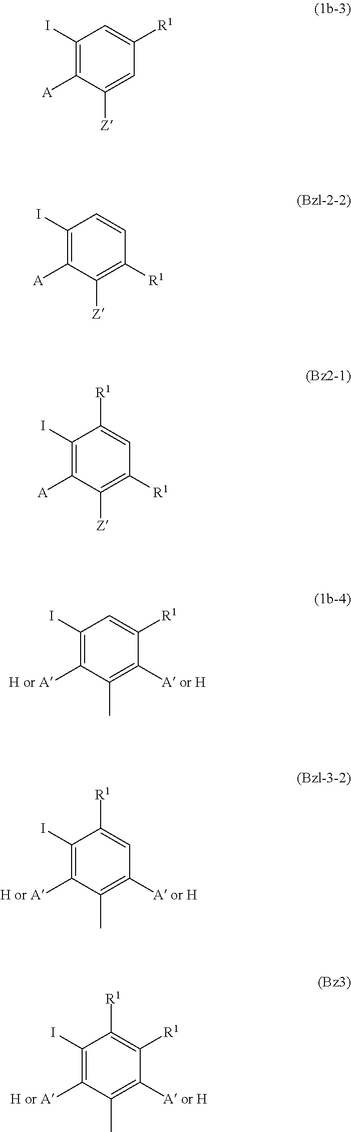
wherein I, R, R¹, A, and A′ are as defined above; and
Z′ is I, R¹, or H.

45. The compound according to claim 41, wherein R¹ is a hydroxy group, a carboxyl group, an ester group, or a hydroxyalkyl group.

46. The compound according to claim 45, wherein the compound is represented by any one of the following formulas: 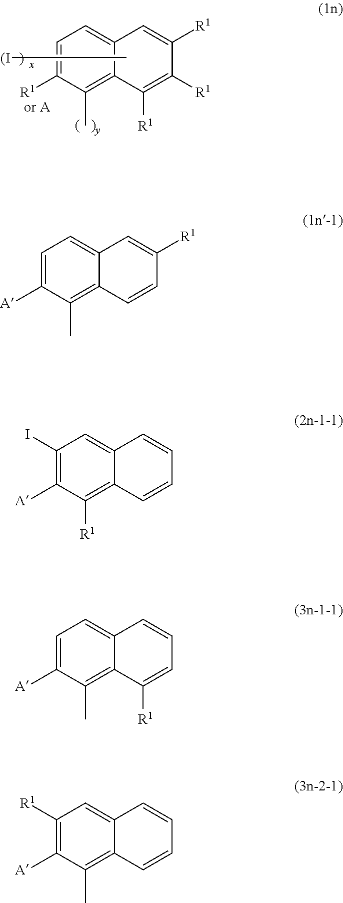
wherein I, Z, R¹, R″, A, x, and y are as defined above.

47. The compound according to claim 42, wherein R¹ is a hydroxy group, a carboxyl group, an ester group, or a hydroxyalkyl group.

48. The compound according to claim 47, wherein the compound is represented by any one of the following formulas: 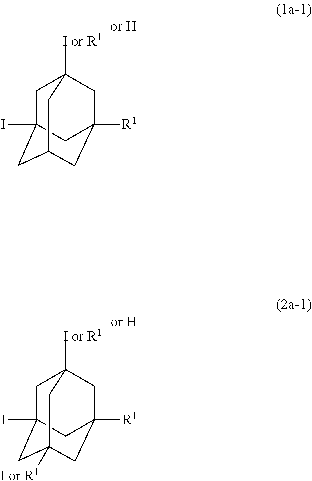
wherein I and R¹ are as defined above.

49. The compound according to claim 1, wherein the compound expresses a sensitizing effect in radiation irradiation of a composition for lithography.

50. The compound according to claim 1, wherein the compound is used for lithography.
